(19) United States
(12) Patent Application Publication
(10) Pub. No.: US 2025/0137008 A1
(43) Pub. Date: May 1, 2025

(54) MODIFIED ION CHANNELS

(71) Applicant: UNIVERSITY OF GLASGOW, Glasgow (GB)

(72) Inventor: Mike BLATT, Glasgow (GB)

(21) Appl. No.: 18/835,843

(22) PCT Filed: Feb. 8, 2023
(86) PCT No.: PCT/EP2023/053078

§ 371 Date: Aug. 5, 2024

(30) Foreign Application Priority Data
Feb. 8, 2022 (LU) 501409

(51) Int. Cl. C12N 15/82; C07K 14/415

(52) U.S. Cl. CPC C12N 15/8273; C07K 14/415; C12N 15/8205

(57) ABSTRACT

The invention relates to methods of increasing stomatal function in plants, which leads to an increase in water use efficiency and ultimately an increase in biomass and yield. In particular the methods of the invention relate to modifying stomatal voltage-gated potassium channels to accelerate stomatal conductance and kinetics. Also described are plants expressing these modified channels and well as methods of producing such plants.

### FIELD OF THE INVENTION

The invention relates to methods of increasing stomatal function in plants, which leads to an increase in water use efficiency and ultimately an increase in biomass and yield. In particular the methods of the invention relate to modifying stomatal voltage-gated potassium channels to accelerate stomatal conductance and kinetics. Also described are plants expressing these modified channels and well as methods of producing such plants.

### BACKGROUND OF THE INVENTION

Globally, water usage has increased 6-fold in the past 100 years, twice as fast as the human population, and is expected to double again before 2030, driven mainly by irrigation and agriculture. In the UK alone, irrigation has risen 10-fold in the past 30 years and this trend is expected to continue.

Stomata are pores in the leaf epidermis that form between pairs of guard cells. They allow CO₂ uptake for photosynthetic carbon assimilation at the expense of water loss via transpiration. As such, they provide the major route for gaseous exchange between the interior of the leaf and the atmosphere and can limit photosynthetic rates by 50% or more when demand exceeds water supply. Stomata exert a major control on the water and carbon cycles of the world, and their activity in crops has been a key factor in global atmospheric modelling and weather prediction for over a quarter of a century. Stomatal aperture is controlled by guard cell turgidity, which responds to changes in atmospheric CO₂ concentration, light, atmospheric relative humidity, and abscisic acid, thereby regulating plant water use.

To date, efforts to improve plant water use efficiency have focused largely on reducing stomatal density, despite its implicit penalty in carbon assimilation. Approaches that circumvent the carbon:water trade-off pose greater challenges but also provide much promise. In particular, accelerating the kinetics of stomatal opening and closing could be used to promote carbon assimilation under high light intensities, while maintaining plant water status when carbon demand is low.

Much research to date has focused on enhancing water use efficiency (WUE) by reducing stomatal densities, an approach that decreases transpiration but can also reduce CO₂ availability for photosynthesis and slow plant growth. Some studies have shown that altering the populations of ion pumps and channels will affect stomatal conductance and photosynthesis, but commonly at the expense either of WUE or of carbon assimilation.

There therefore exists a need to accelerate stomatal kinetics to reduce transpiration rates from the stomata without affecting photosynthesis. This will be particularly important in crops with slow stomatal kinetics, and will ultimately lead to an increase in growth and/or yield in these crops.

The present invention addresses this need.

### SUMMARY OF THE INVENTION

We have shown that by introducing mutations into stomatal voltage-gated potassium channels, we can alter channel clustering and the voltage- and K⁺-dependencies for K⁺ flux, thereby enhancing guard cell membrane transport and accelerating stomatal movements. These findings underscore the gains possible by ‘tuning’ the gating of channels native to the plant to improve WUE and carbon assimilation, thus circumventing the often conflicting demands in conserving water while ensuring photosynthetic assimilation for growth.

In one aspect of the invention, there is provided a genetically altered plant, plant part or plant cell, wherein the plant is characterised by at least one mutation in the N-terminal domain of a stomatal voltage-gated potassium channel.

In another aspect of the invention there is provided a method of increasing at least one of growth, yield, drought tolerance, water use efficiency and/or carbon assimilation in a plant, the method comprising introducing at least one mutation into the N-terminal domain of a stomatal voltage-gated potassium channel.

### DESCRIPTION OF THE FIGURES

The invention is further described in the following non-limiting figures:

FIG. 1. GORK K⁺ channels interact through the cytosolic N-termini of the voltage sensor domains

(a) Schematic of Kv channel subunit structure (above) with the six, transmembrane α-helices (S1-S6), intervening loops (L1-L4) and chimera exchange points indicated. Four homologous subunits assemble around a central pore to give a functional channel with the voltage-sensor domains (VSDs) forming an outer ring within the plane of the membrane (\textcircled{1}). Neighboring channels may come together bridged by interactions between VSDs (\textcircled{2}), but interactions between subunits will also occur between individual subunits within the channel assembly (\textcircled{1}). Exploring interactions between VSDs therefore requires to isolate one VSD component (\textcircled{3}). (b) Selective binding occurs between GORK and the GORK VSD (gVSD) but is not evident with either KAT1 or its VSD (kVSD). Cartoons (left) indicate the bait and prey structures. One of three independent experiments, all yielding similar results. Yeast mating-based split-ubiquitin assay for interaction of the Nub-X channel and VSD fusions, including controls ([−], NubG; [+], NubI), with the GORK-Cub bait fusion. Yeast diploids dropped at 1.0 and 0.1 OD₆₀₀ spotted (left to right) on complete synthetic medium without Trp, Leu, Ura and Met (CSM_{-LTUM}) to verify mating, on CSM without Trp, Leu, Ura, Ade, His and Met (CSM_{-LTUMAH)} to verify adenine- and histidine-independent growth, and with Met additions to suppress bait expression. (c) Chimeras of the GORK VSD (gVSD) and KAT1 VSD (kVSD) interact with the full-length GORK channel provided the VSD prey includes the gVSD N-terminus. Cartoons (left) indicate the bait and prey structures. One of three independent experiments, all yielding similar results. Yeast mating-based split-ubiquitin assay for interaction of the Nub-X VSD and VSD chimera fusions, including controls ([−], NubG; [+], NubI), with the GORK-Cub bait fusion as in (b).

FIG. 2. Alternation of charges defines GORK VSD interactions

Mutation of the GORK VSD N-terminus supports interaction provided the alternation of charges is retained. (a) Alignment of the N-terminal cytosolic domains GORK homologs highlights an alternation of positive and negative charged regions. Sequences (NCBI identifiers) from Arabidopsis thaliana (CAC17380.1), Brassica napus (XP_013656069.1), Arabidopsis relative Tarenaya hassleriana (XP_010553885.1), oil palm Elaeis guineensis (XP_010905454.2), pepper Capsicum annuum (KAF3658364.1), wheat Triticum aestivum (XP_044442180.1), and rice Oriza sativa (XP_015644419.1). Charged residues (positive, blue; negative, yellow) are highlighted.

(b) Alignment of the GORK and KAT1 N-termini. Negative and positive charged residues were exchanged in gVSD^{PN} and gVSD^{NP}, respectively, and are substituted with Ala in gVSD^{A}. His residues were included as these carry a substantial positive charge at neutral pH. All charged residues were exchanged in gVSD^{PNNP}. (c) One of three independent experiments, all yielding similar results testing the residue mutants in (b) for interaction with the full-length GORK channel here cross-referenced by color coding (left). Yeast mating-based split-ubiquitin assay for interaction of the Nub-X VSD fusions, including controls ([−], NubG; [+], NubI), with the GORK-Cub bait fusion. Yeast diploids dropped at 1.0 and 0.1 OD₆₀₀ spotted (left to right) on complete synthetic medium without Trp, Leu, Ura and Met (CSM_{-LTUM}) to verify mating, on CSM without Trp, Leu, Ura, Ade, His and Met (CSM_{-LTUMAH}) to verify adenine- and histidine-independent growth, and with Met additions to suppress bait expression.

FIG. 3. Non-interacting channel mutations suppress GORK clustering when stably expressed in Arabidopsis

(a) Confocal analysis of GFP channel fusions (GFP), chlorophyll fluorescence (Chlpst), and corresponding brightfield (Bright) images from gork Arabidopsis leaf epidermis transformed with wild-type GORK-GFP (GORK^{wt}), GFP fusions of the non-interacting mutations GORK^{kN-gS1-L4} and GORK^{NP}, and the interacting GORK^{PNNP} mutant under control of the guard cell pGC1 promoter. Representative image sets from leaves infiltrated²² with 0.1 mM and subsequently with 50 mM K⁺. Scale bar, 10 μm. (b) Relative standard deviation (RSD) of GFP distributions. RSD calculated from the GFP fluorescence measured along a 2 μm-wide line traced over the periphery of 10 guard cells selected at random in n>6 independent experiments with each construct. Data are means ±SEM for 0.1 (●) and 50 mM (◯) K⁺ with means from individual experiments indicated by the smaller grey symbols. Note the logarithmic scale. Significance at P<0.02 is indicated by lettering.

FIG. 4. Non-interacting GORK channel mutations express functional K⁺ channels with displaced midpoint voltages

Representative current traces and steady-state current-voltage (IV) curves recorded under voltage clamp from guard cells of wild-type Arabidopsis (a) and guard cells of the gork mutant background complemented with GFP fusions of wild-type GORK (GORK^{wt}, b), the non-interacting mutations GORK^{kN-gS1-L4} (c) and GORK^{NP} (d), and the interacting GORK^{PNNP} mutation (e) under control of the guard cell pGC1 promoter. IV curves are means ±SE of n>6 independent experiments, each recording comprising measurements from individual guard cells superfused successively with 1 (filled circles), 10 (grey circles), and 30 mM (open circles) K⁺ outside, cross-referenced by symbol. Scale bar: horizontal, 1 s; vertical, 100 ρA cm⁻². Lines are joint, least-squares fittings to Eqn [1]. Fittings yielded a common δ of 1.82±0.04. Means ±SE for V_{1/2} from these fittings, summarized in (f), show a significant negative shift for each of the non-interacting mutant channels. Significant differences at P<0.05 across all K⁺ concentrations are indicated (letters, right).

FIG. 5. Non-interacting GORK channel mutations accelerate stomatal closure and enhance water use efficiency

(a) Stomatal conductance (gₛ) responses to a 100 min light step of 200 μmol m⁻²s⁻¹ PAR following a 1-h dark period (above). Simulation outputs (left) are for wild-type (wt), GORK with a −30 mV displacement in V_{1/2} (mGORK; see FIG. 4) and for the gork nul mutant. Experimental data (right) are measurements from the same plants as in FIGS. 3 and 4 and are means ±SE of n>5 independent experiments on wild-type (wt), the nul mutant background (gork), and the same background expressing the GORK^{NP} mutation. (b) Closing rate constants for the simulations (diamonds) and experimental measurements including those in (a). Rate constants were determined from fittings to a first order exponential function and are shown for each measurement (grey circles) and as means ±SE of these data (black circles). Significant differences are indicated (letters, below). (c) Opening halftimes for the simulations (diamonds) and experimental measurements including those in (a). Halftimes are shown for each measurement (grey circles) and as means ±SE of these data (black circles). Significant differences are indicated (letters, below). (d) Representative wild-type, gork mutant and GORK^{wt}, GORK^{kN-gS1-L4}, GORK^{NP} and GORK^{PNNP} complemented gork mutant plants after 5 wk growth under fixed and varying light regimes (see Methods). (e,f) Rosette areas (e), dry biomass and water use efficiencies (f) for all plants, including the plants shown in (d). Small circles are individual plants (open, fixed light; filled, varying light); large circles (open, fixed light; filled, varying light) are means ±SE for each set of plants (wild-type n=11 plants; mutant and complemented n>8 plants). Note the separate WUE scales for plants grown under fixed and varying light. Significant differences are indicated (letters, below).

FIG. 6. A mechanism coupling GORK clustering and gating through a binding exchange of the VSD N-terminus

We start with the assumption that elevating K⁺ outside facilitates transition from the open channel to a ‘locked closed’ state, similar to the SKOR K⁺ channel (left: grey arrow=reduced, black arrow=enhanced bias)²⁴. As shown, this closed state associates with a shift from binding of the VSD N-terminus (red) with neighboring channels and clustering (above) at low K⁺ to internal binding that supports the ‘locked closed’ state and sensitizes channel gating to K⁺ (below). Mutating the VSD N-terminus yields a channel (mGORK) impaired in both clustering and internal binding, the latter suppressing the ‘locked closed’ state K⁺ inhibition of gating. The mGORK channel thus retains gating characteristics that are closer to the channel clusters, but with a reduced sensitivity to gating inhibition by K⁺, that enhances K⁺ flux and hence stomatal kinetics.

FIG. 7: Truncating the GORK voltage sensor domain beyond the first 23 residues of the N-terminus suppresses interactions with the full-length channel.

FIG. 8: Alanine-scanning mutagenesis of the GORK voltage sensor domain N-terminus fails to identify a binding motif.

GORK VSD (gVSD) N-terminal site mutations by residue (m) were used as Nub-X preys for interaction with the full-length GORK channel Y-Cub fusion in the yeast mating-based split-ubiquitin assay. Controls ([−], NubG; [+], NubI) included below. One of two independent experiments, both yielding similar results. Yeast diploids dropped at 1.0 and 0.1 OD600 spotted (left to right) on complete synthetic medium without Trp, Leu, Ura and Met (CSM-LTUM) to verify mating, on CSM without Trp, Leu, Ura, Ade, His and Met (CSM-LTUMAH) to verify adenine- and histidine-independent growth, and with Met additions to suppress bait expression.

FIG. 9: Non-interacting channel mutations suppress GORK clustering when expressed in tobacco.

(a) Confocal analysis of GFP channel fusions (GFP), chlorophyll fluorescence (Chlpst), and corresponding brightfield (Bright) images from tobacco epidermis transiently transformed with wild-type GORK-GFP (GORKwt), GFP fusions of the non-interacting mutations GORK^{kN-gS1-L4} GORK^{PN} and GORK^{NP}, and the interacting GORK^{PNNP} mutant under control of the pUBQ10 promoter. Image sets collected while superfusing with 0.1 mM and subsequently with 50 mM K+. Scale bar, 10 μm. (b) Relative standard deviation (RSD) of GFP distributions. RSD calculated from the GFP fluorescence measured along a 1 μm-wide line traced around the periphery of 10 epidermal cells selected at random in n>6 independent experiments with each construct. Data are means ±SE for 0.1 (black data points) and 50 mM (white data points) K+ with means from individual experiments indicated by the smaller grey symbols. Note the logarithmic scale. Significant differences at P<0.02 is indicated by lettering.

FIG. 10: Non-interacting GORK channel mutations express functional K+ channels with displaced midpoint voltages.

Representative current traces and mean steady-state current-voltage (IV) curves recorded under voltage clamp from oocytes expressing the wild-type channel (GORK^{wt}, a), the non-interacting mutations GORK^{kN-gS1-L4} (b), GORK^{NP} (c), GORK^{PN} (d), and the interacting mutant GORK^{PNNP} (e). IV curves are means ±SE of n>6 independent experiments, each recording comprising measurements from individual oocytes superfused successively with 1 (black circle), 3 (white circle), 10 (black triangle), 30 (white triangle), and 60 mM (black nabla) K+ outside, cross-referenced to the traces by symbol. Scale bars: horizontal, 1 s; vertical, 5 μA. Means ±SE for V1/2 from these fittings, summarized in (f), show a highly significant shift to more negative voltages and higher K+ for each of the non-interacting mutant channels. Significant differences at P<0.02 across all K+ concentrations are indicated (letters, right).

FIG. 11: OnGuard3 modelling predicts an enhanced capacity for K+ flux through the mutant GORK channel

(a) Model current-voltage (IV) curves for the wild-type (wt, black line) and the GORK channel mutated to displace the K+-dependence of gating by −30 mV (mGORK, grey line). IV curve for the KAT current (dashed line) included for reference. Curves correspond to the channel current with 10 mM K+ outside. Inset: IV curves plotted on an expanded current scale. Shading indicates the enhanced capacities for K+ efflux (yellow) and K+ influx (green) at voltages positive and negative of EK, respectively. (b) Model K+ flux in the wild-type (wt, black line) and mutant GORK (mGORK, grey line) as in (a) over time with a light step from 10 to 200 μmol m-2 s-1 PAR (above). Shading indicates the enhanced K+ efflux (yellow) and K+ influx (green).

### DETAILED DESCRIPTION OF THE INVENTION

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

The practice of the present invention will employ, unless otherwise indicated, conventional techniques of plant biology, microbiology, tissue culture, molecular biology, chemistry, biochemistry and recombinant DNA technology, bioinformatics which are within the skill of the art. Such techniques are explained fully in the literature.

As used herein, the words “nucleic acid”, “nucleic acid sequence”, “nucleotide”, “nucleic acid molecule” or “polynucleotide” are intended to include DNA molecules (e.g., cDNA or genomic DNA), RNA molecules (e.g., mRNA), natural occurring, mutated, synthetic DNA or RNA molecules, and analogs of the DNA or RNA generated using nucleotide analogs. It can be single-stranded or double-stranded. Such nucleic acids or polynucleotides include, but are not limited to, coding sequences of structural genes, anti-sense sequences, and non-coding regulatory sequences that do not encode mRNAs or protein products. These terms also encompass a gene. The term “gene” or “gene sequence” is used broadly to refer to a DNA nucleic acid associated with a biological function. Thus, genes may include introns and exons as in the genomic sequence, or may comprise only a coding sequence as in cDNAs, and/or may include cDNAs in combination with regulatory sequences.

The terms “polypeptide” and “protein” are used interchangeably herein and refer to amino acids in a polymeric form of any length, linked together by peptide bonds.

The aspects of the invention involve recombination DNA technology and exclude embodiments that are solely based on generating plants by traditional breeding methods.

For the purposes of the invention, a “genetically altered plant” or “mutant plant” is a plant that has been genetically altered compared to the naturally occurring wild type (WT) plant.

In a first aspect of the invention there is provided a genetically altered plant, part thereof or plant cell wherein the plant, part thereof or plant cell is characterised by at least one mutation in a stomatal voltage-gated potassium channel.

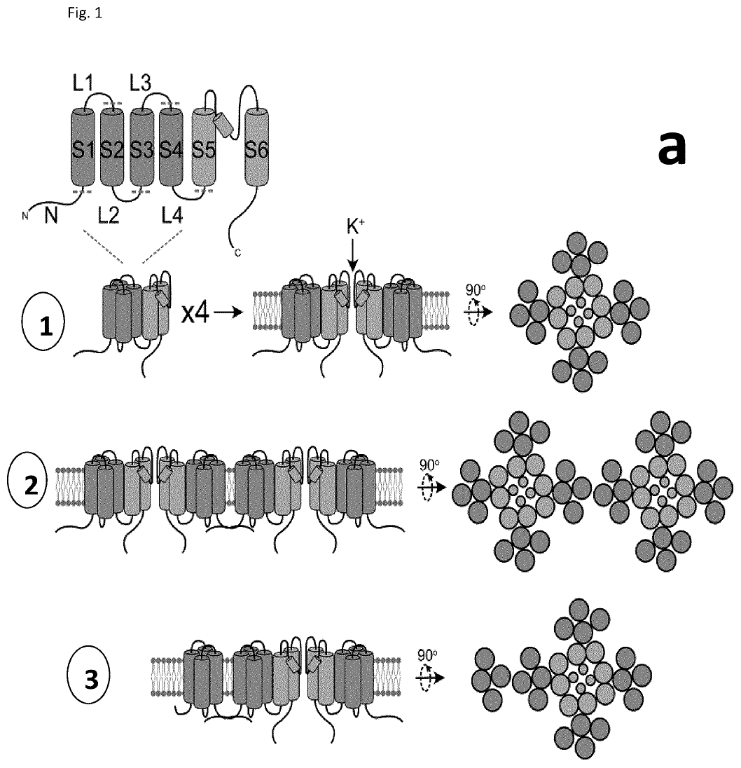

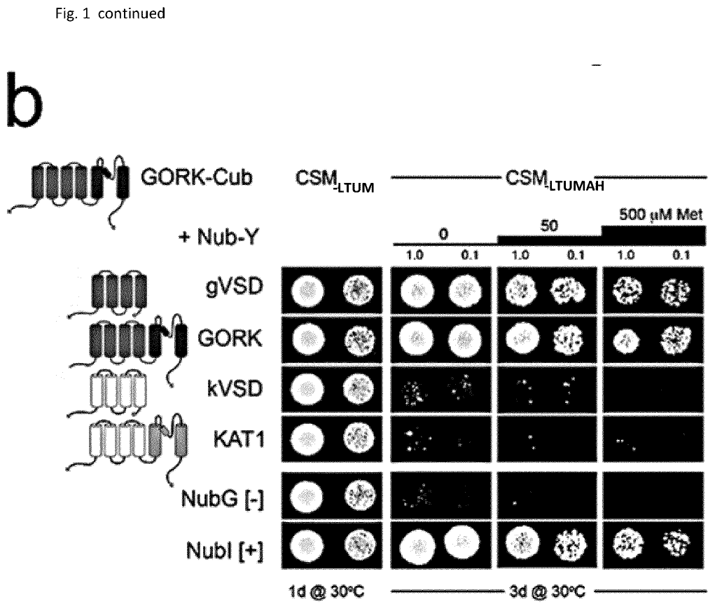

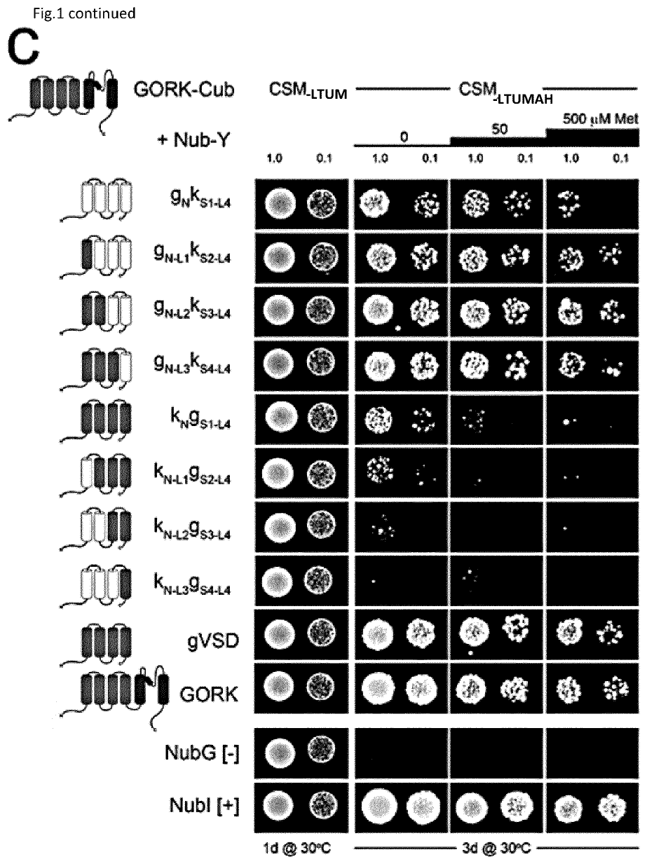

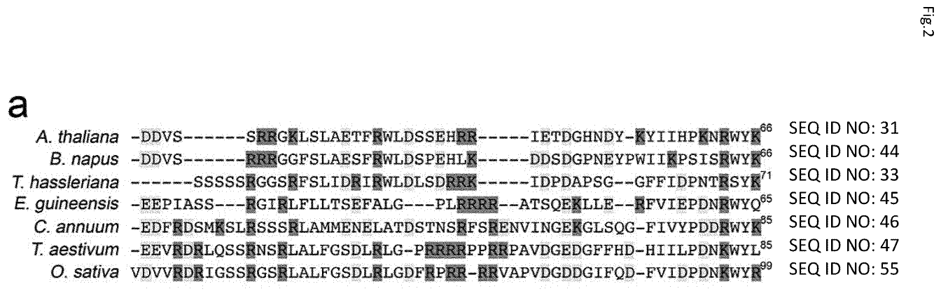

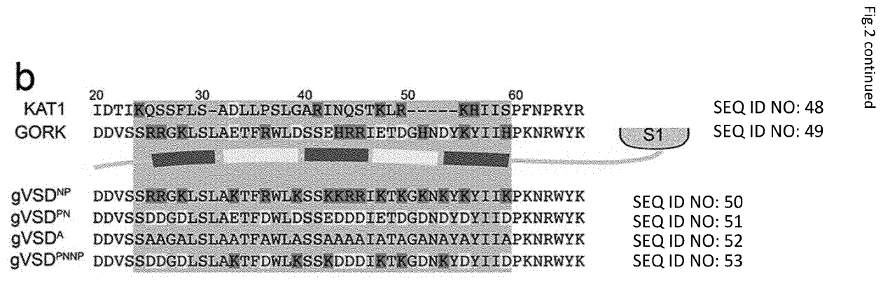

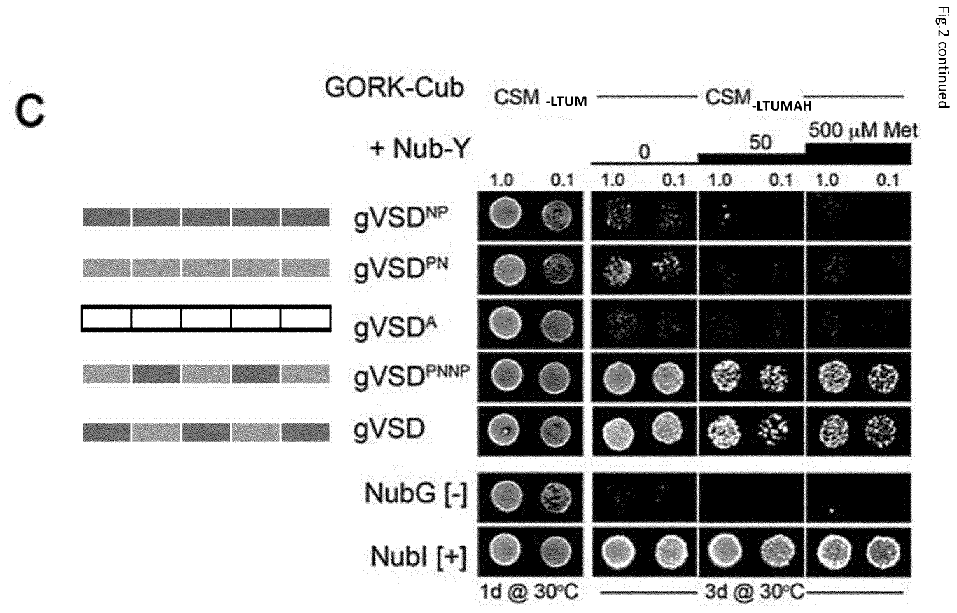

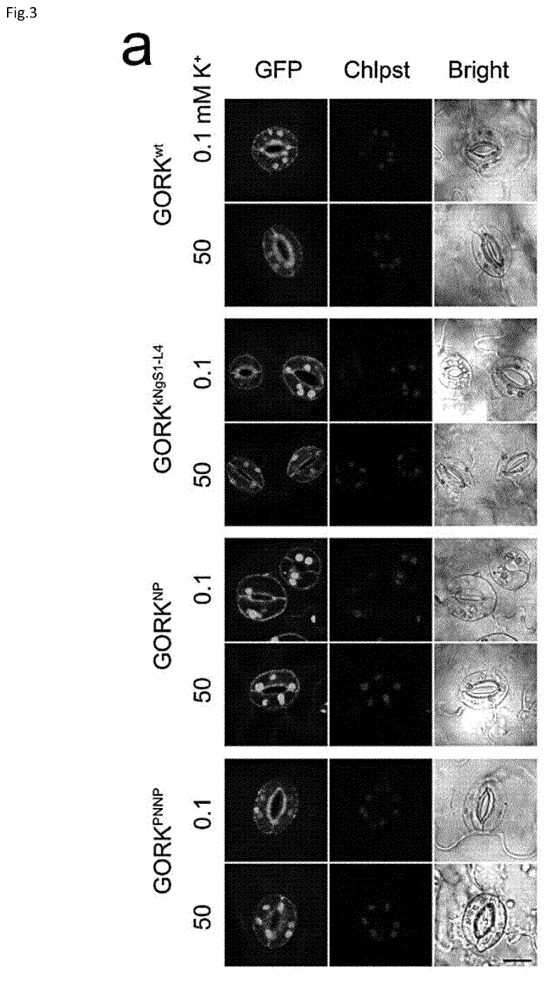

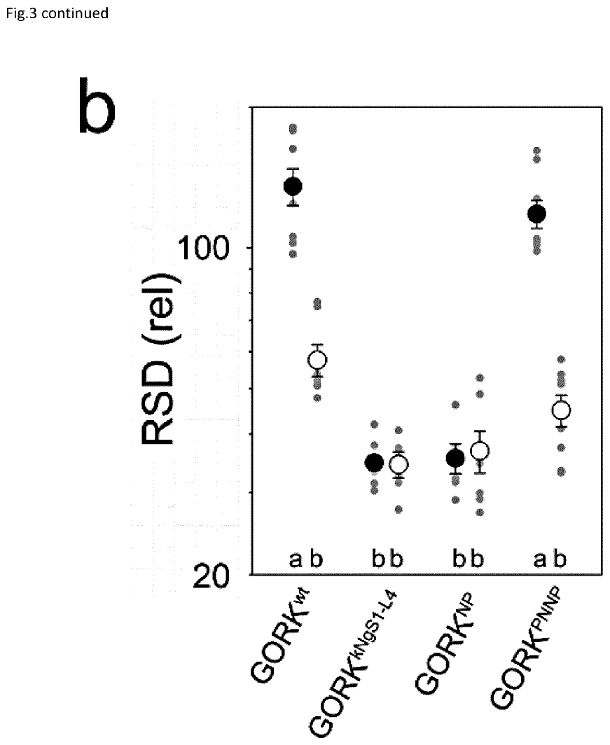

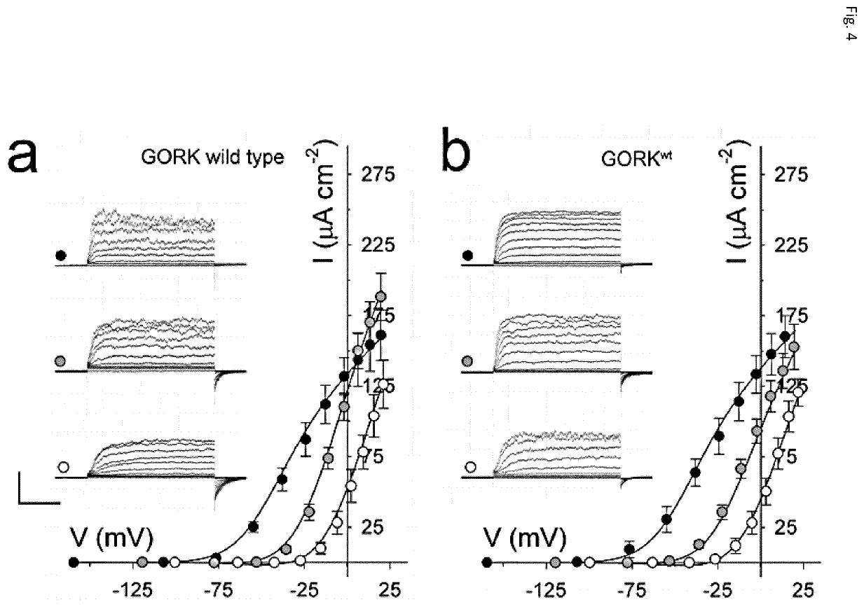

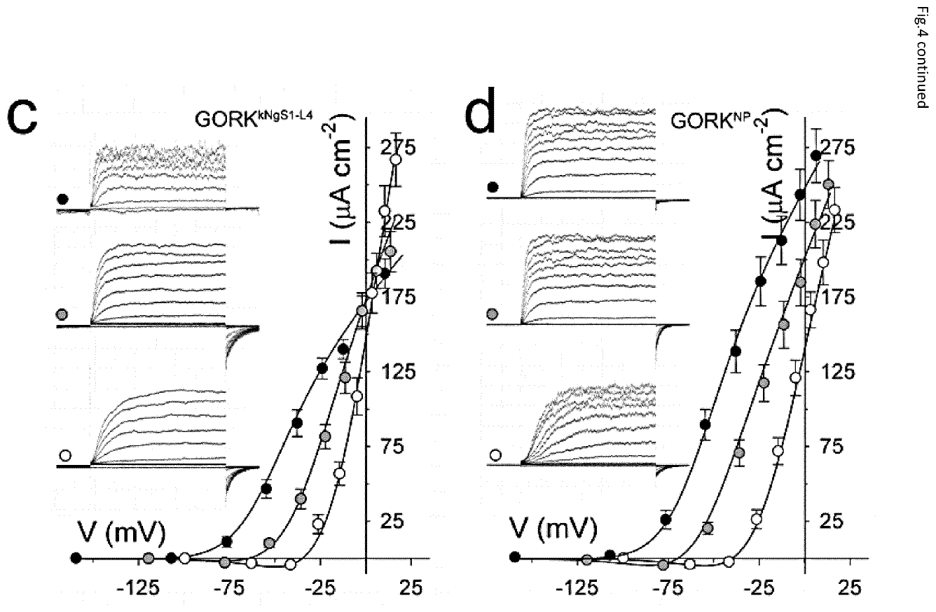

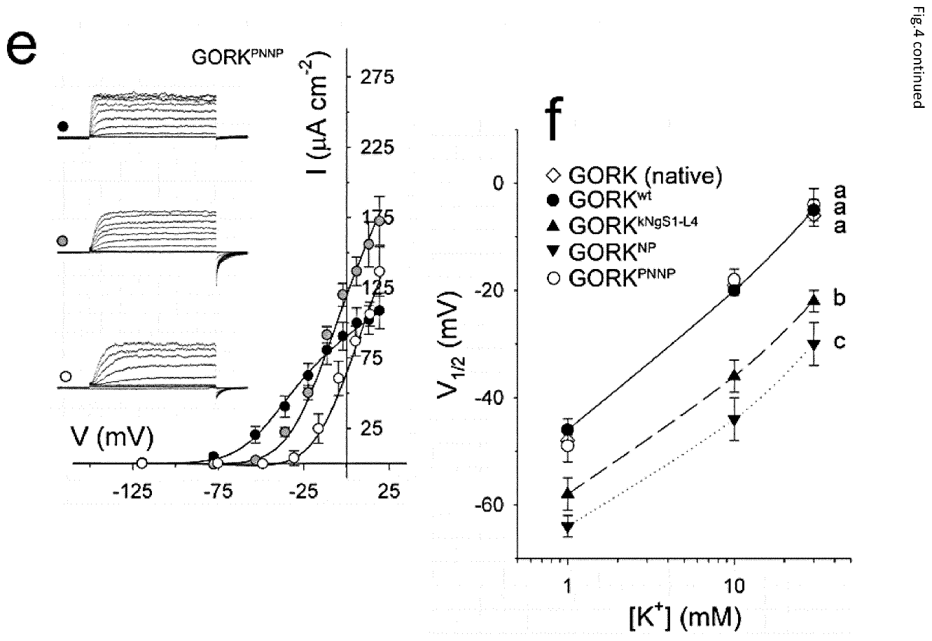

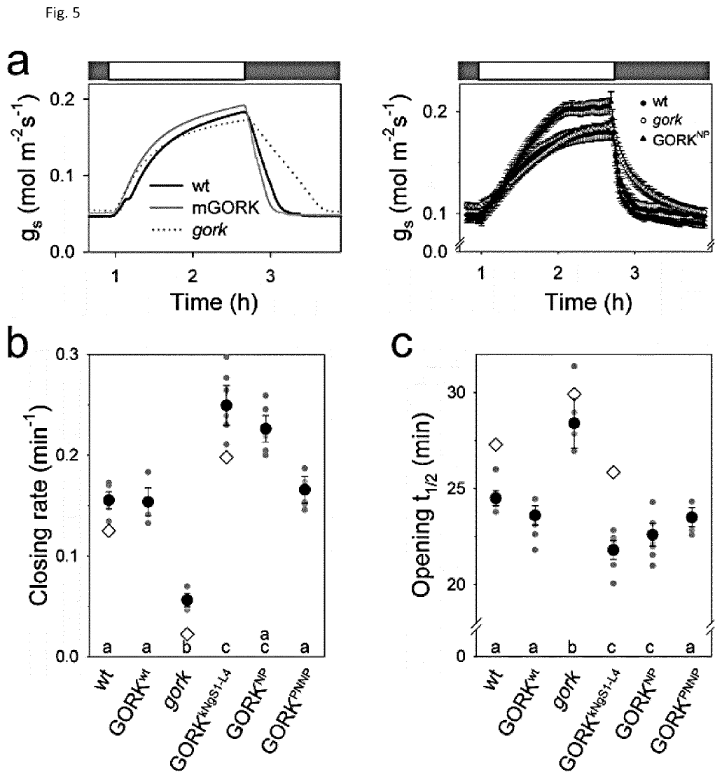

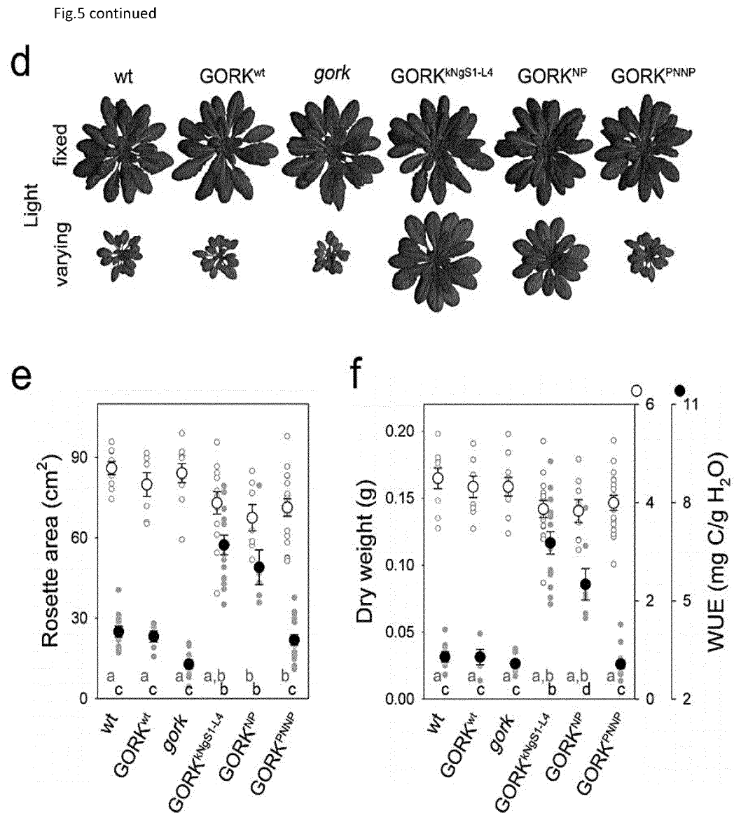

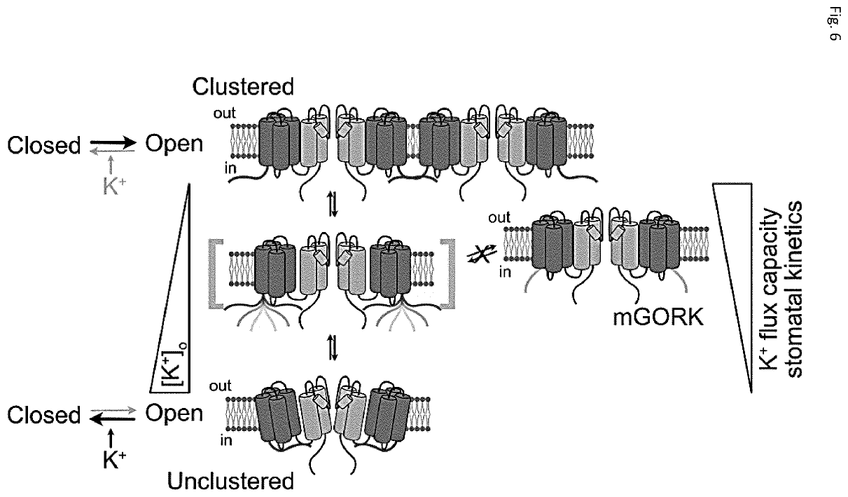

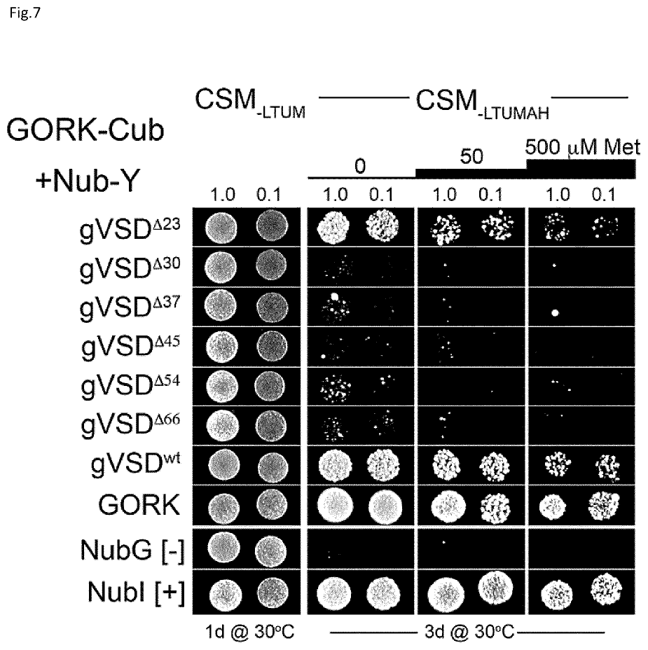

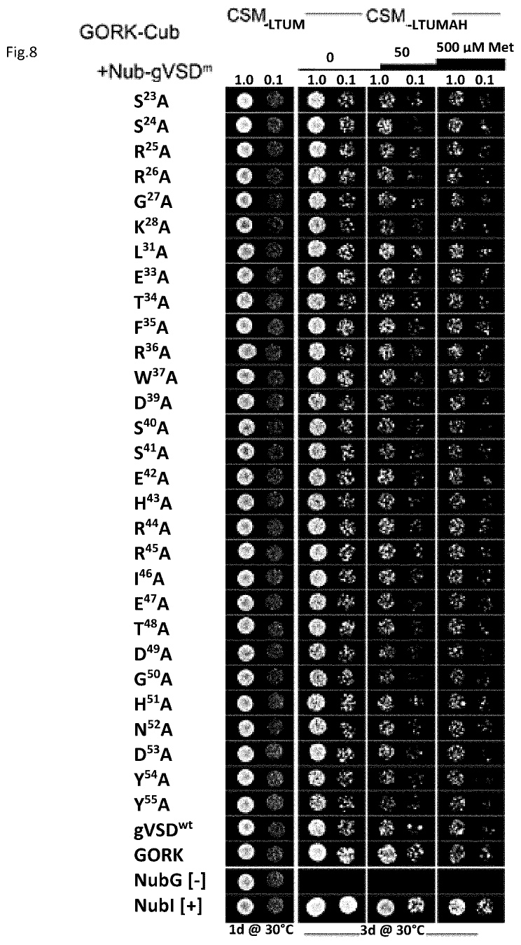

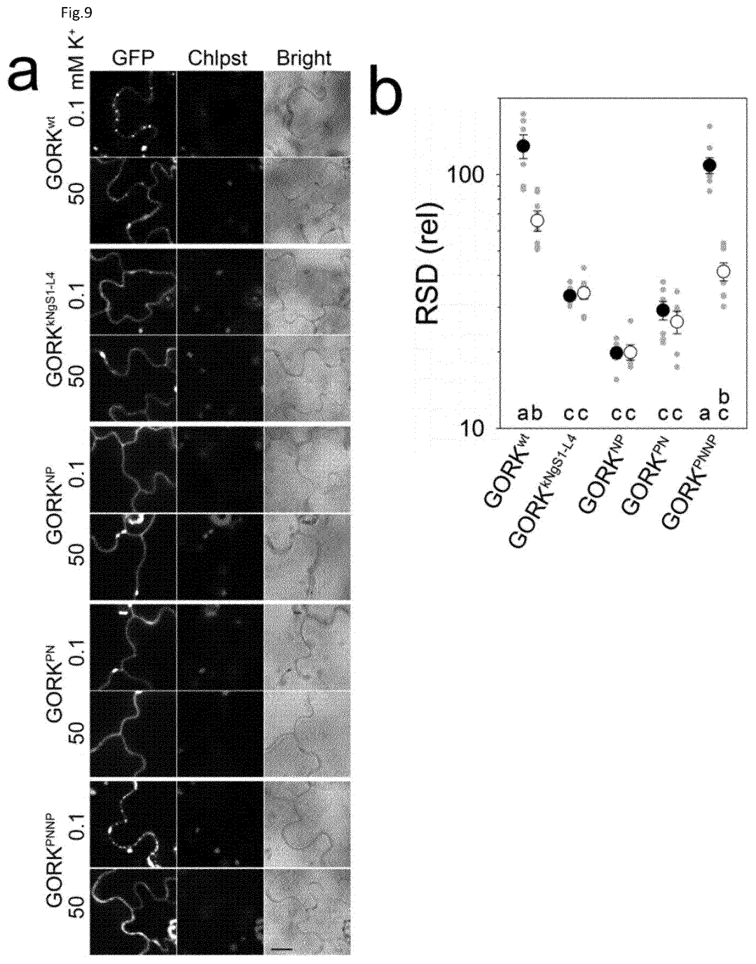

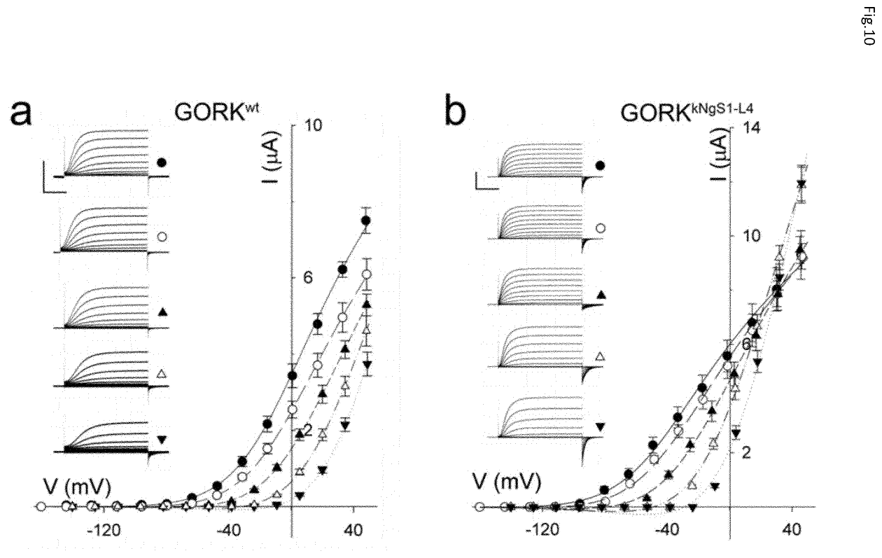

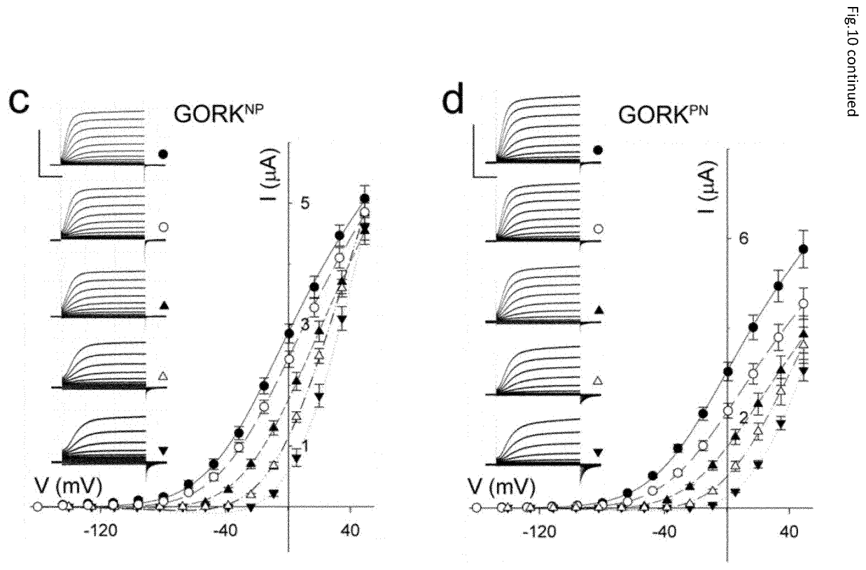

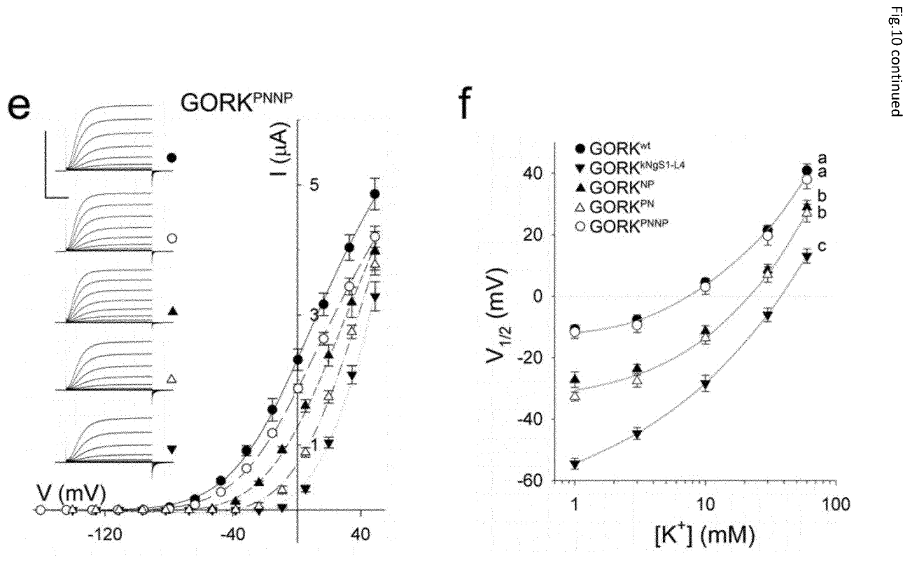

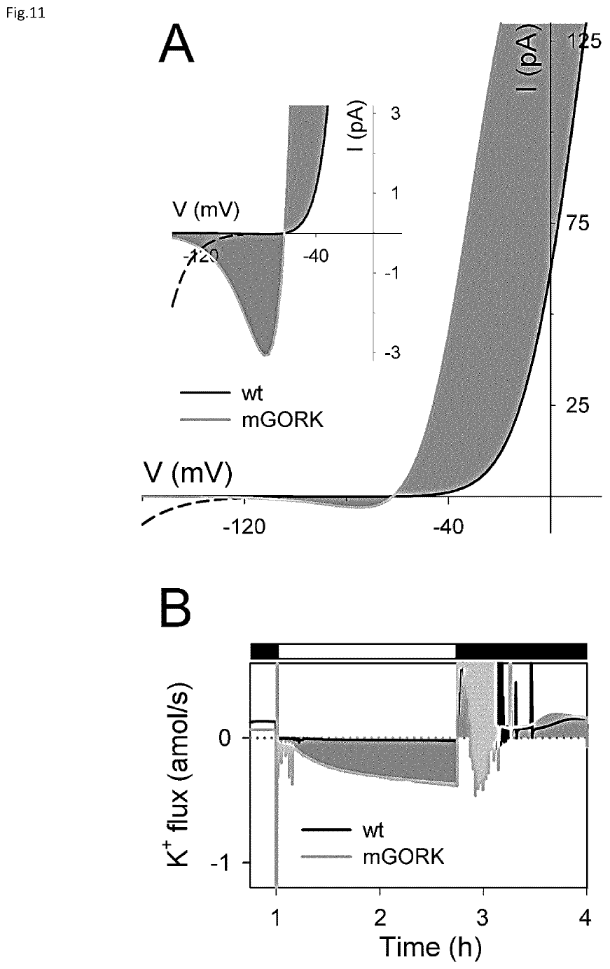

Voltage-sensitive K⁺ channels—including the outward-rectifying channels GORK and closely-related SKOR as well as the inward-rectifying channel KAT1—belong to the superfamily of so-called Kv channels that are found across all phyla. These proteins form functional channels as tetramers, each protein subunit comprising six transmembrane α-helices with cytosolic N- and C-termini (as shown in FIG. 1A). The first four α-helices (S1-S4) of the subunit form a semi-autonomous voltage-sensor domain (VSD), a structure that is highly conserved among voltage-gated channels. The last two α-helices (S5-S6) line a water-filled pore through the membrane within the centre of the tetrameric channel assembly. The pore of P region between S5 and S6 alpha-helices has a short pore helix with a highly conserved TxGYGD (SEQ ID NO: 54) domain serving as the selective filter for potassium. The pore-lining α-helices connect to the VSD through a cytosolic linker that ensures that conformational changes between the VSD and the pore domains occur in ‘lockstep’. These channels open and close, in a process called “gating” in response to membrane voltage, as well as the extracellular potassium concentration. The VSD itself incorporates a network of charges that, with a change in voltage, drive VSD conformation and alter its hydration surface as it moves part-way across the membrane, thereby opening the channel pore. GORK and SKOR assembles clusters at submillimolar K+ that reversibly disassembles when K+ is raised to 10 mM and higher concentrations; additionally, clustering is inhibited by the K+ channel blocker Ba⁺ that enters and lodges in the channel pore, preventing K+ permeation and blocking conformational relaxation to the closed state. Thus, it is believed that clustering is connected to gating and that both are characteristics intrinsic to the GORK and SKOR protein itself. Within the plane of the membrane, the VSDs form an outer ring around the channel pore (as shown in FIG. 1A). We have reasoned that clustering between GORK channels within the membrane entails inter-channel interactions between VSDs.

The following embodiments apply to all aspects of the invention.

In one embodiment, the stomatal voltage-gated potassium channel is an outward-rectifying potassium efflux channel (also known as a “Kₒᵤₜ channel”). Kₒᵤₜ channels respond to the extracellular K⁺ concentration. Increasing the concentration of extracellular K⁺ suppresses channel opening in a voltage-dependent manner. In contrast, a low concentration of extracellular K⁺ promotes channel opening. In particular, activation of Kₒᵤₜ channels is dependent on membrane depolarization, such that the channels open when the membrane potential shifts to more positive voltages, usually higher than the K⁺ equilibrium voltage (Eₖ). This allows K⁺ release. In other words, Kₒᵤₜ channels open when membrane voltages are higher than Eₖ so that the driving force on K⁺ is outward.

In one embodiment, the potassium channel is a GORK (Guard cell Outward Rectifying Potassium (K⁺)) channel. In another embodiment, the potassium channel is a SKOR (Outward rectifying Shaker-type K⁺) channel. These channels share similar alternations in charge along their N-termini, as shown in FIG. 2a.

In a further embodiment, the amino acid sequence of the GORK channel is selected from one of SEQ ID NOs: 1, 3 and 5 or a functional homologue or variant thereof. In a further embodiment, the nucleic acid sequence of the GORK channel is selected from one of SEQ ID NOs: 2, 4 and 6 or a functional variant or homologue thereof.

In another embodiment, the amino acid sequence of the SKOR channel is selected from one of SEQ ID NOs 7, 9, 11, 13, 15, 17, 19 and 21 or a functional variant or homologue thereof. In a further embodiment, the nucleic acid sequence of the SKOR channel is selected from one of SEQ ID NOs: 8, 10, 12, 14, 16, 18, 20 or 22 or a functional variant or homologue thereof.

In a further embodiment, the sequence of the stomatal voltage-gated potassium channel is selected from one of the below sequences in Table 1.

TABLE 1
stomatal voltage-gated potassium channel sequences from different organisms.

| | | | Organism- | SEQ ID |
|---|---|---|---|---|
| Organism | UniProt | NCIB/GenBank | specific databases | NO: |
| Arabidopsis thaliana | Q94A76 | CAC17380.1 | AT5G37500 | SEO ID |
| | | | | NO: 1 |
| Brassica napus (Rape) | A0A078H3V4 | XP_013656069.1 | | SEQ ID |
| | | | | NO: 3 |
| Tarenaya hassleriana | | XP_010553885.1 | | |
| Elaeis guineensis (Oil palm) | A0A619QAA3 | XP_010905454.2 | | SEQ ID |
| | | | | NO: 7 |
| Raphanus sativus (Radish) | A0A6J0JE53 | XP_018433301.1 | | SEQ ID |
| | | | | NO: 5 |
| Capsicum annuum | A0A1U8GYE0 | KAF3658364.1 | | |
| (peppers) | | | | |
| Hibiscus syriacus | A0A6A2ZB50 | XP_039017376.1 | | |
| (Rose of Sharon) | | | | |
| Triticum aestivum (Wheat) | A0A3B6RCP3 | XP_044442180.1 | | |
| Oryza sativa (Rice) | Q653P0 | XP_015644419.1 | | SEQ ID |
| | | | | NO: 9 |
| Gynandropsis gynandra | — | | | |
| 12581.1 (C4) | | | | |
| Zea mays (maize, C4) | A0AID6LW78 | PWZ19722.1 | | |
| Panicum miliaceum | A0A3L6PAE8 | RLM54412.1 | | |
| (Proso millet, C4) | | | | |
| Sorghum vulgare | A0A194YID1 | XP_021305239.1 | | |
| (Sorghum bicolor, C4) | | | | |

In one embodiment, the plant, part thereof or plant cell comprises at least one mutation in one of the above sequences.

In one embodiment, the mutation is in the N-terminus of the potassium channel. In one embodiment, the N-terminus comprises the voltage-sensing domain (VSD). By “VSD” is meant the cytosolic N termini, the S1, S2, S3 and S4 transmembrane alpha helices and the cytosolic loops between S1 and S2, between S2 and S3, between S3 and S4 and between S4 and S5. Accordingly, in one embodiment, the one or more mutation are in the VSD.

In a particularly preferred embodiment, the mutation is in the cytosolic N termini. Typically the N termini makes up around the first 100 to 150 amino acids of the potassium channel. Preferably, the at least one mutation is in the N-terminal 70 (i.e. positions 1-70) amino acids of the potassium channel, more preferably within the N-terminal 60 (i.e. positions 1 to 60) amino acids of the potassium channel. In a further preferred embodiment, the mutation is at one or more of positions 20-60, more preferably at one or more of residues at positions 24-59 of the potassium channel.

In a further embodiment, the plant, part thereof or plant cell, may contain at least one mutation in the cytosolic N termini and further, at least one mutations in the S1, S2, and/or S3 loop and/or the S4/S5 linker.

The mutation may be a substitution of one amino acid for another amino acid or a deletion or addition of one or more amino acids. Preferably, the mutation is a substitution mutation. Alternatively, the mutation is a deletion.

In one embodiment, the sequence of the cytosolic N termini comprises SEQ ID NO: 31 or a functional variant or homologue thereof.

(SEQ ID NO: 31)
DDVSSRRGKLSLAETFRWLDSSEHRRIETDGHNDYKYIIHPKNRWYK

In another embodiment, the sequence of the cytosolic N termini comprises one of the following sequences or a functional variant or homologue thereof.

| | |
|---|---|
| Arabidopsis thaliana | 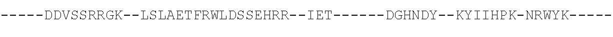 |
| Brassica napus (Rape) | 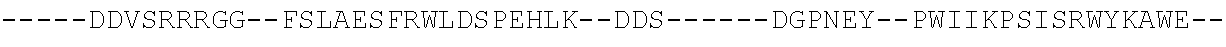 |
| T. hassleriana | 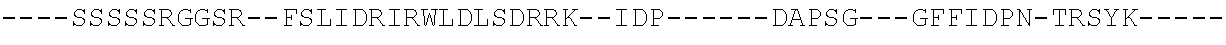 |
| Elaeis guineensis(Oil palm) | 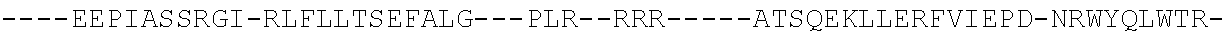 |
| Raphanus sativus (Radish) | 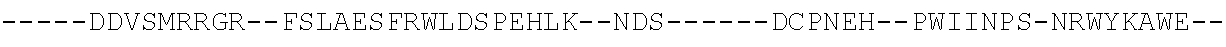 |
| Capsicum annuum (peppers) | 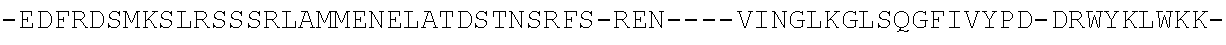 |
| Hibiscus syriacus (Rose of Sharon) | 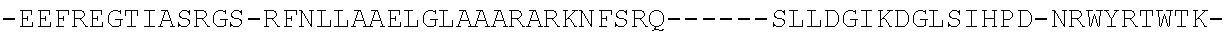 |
| Triticum aestivum (Wheat) | 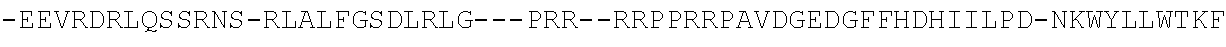 |
| OsKOR1 (Rice) | 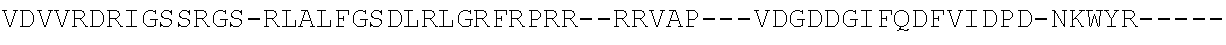 |
| G. gynandra 12581.1(C4) | 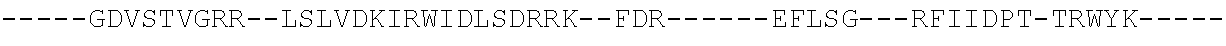 |
| Zea mays (maize, C4) | 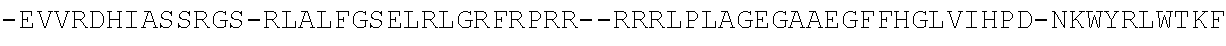 |
| Panicum miliaceum (Proso millet, C4) | 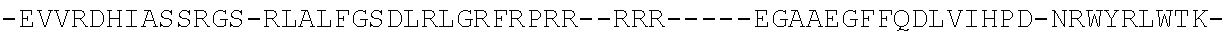 |
| Sorghum bicolor (C4) | 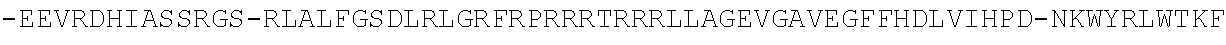 |

| | |
|---|---|
| Arabidopsis thaliana | SEQ ID NO: 31 |
| Brassica napus (Rape) | SEQ ID NO: 32 |
| T. hassleriana | SEQ ID NO: 33 |
| Elaeis guineensis(Oil palm) | SEQ ID NO: 34 |
| Raphanus sativus (Radish) | SEQ ID NO: 35 |
| Capsicum annuum (peppers) | SEQ ID NO: 36 |
| Hibiscus syriacus (Rose of Sharon) | SEQ ID NO: 37 |
| Triticum aestivum (Wheat) | SEQ ID NO: 38 |
| OsKOR1 (Rice) | SEQ ID NO: 39 |
| G. gynandra 12581.1(C4) | SEQ ID NO: 40 |
| Zea mays (maize, C4) | SEQ ID NO: 41 |
| Panicum miliaceum (Proso millet, C4) | SEQ ID NO: 42 |
| Sorghum bicolor (C4) | SEQ ID NO: 43 |

We have found that GORK clustering depends on binding between the VSDs of neighbouring channels—as shown in FIG. 1. It arises through conserved sequences of alternately charged residues that appear to interact in register between the cytosolic N-termini of the VSDs and their complementary binding sites. This pattern of alternately charged amino acids in the cytosolic N-termini is shown in FIG. 2, and is conserved amongst different crops.

We show that mutations affecting the pattern of alternate charges affects clustering, which in turn influences the K⁺-dependence of GORK gating. The results from these mutations proved counterintuitive; it was anticipated that impairing clustering would suppress gating and displace the steady-state IV relations to the right along the voltage axis. Yet, paradoxically, mutations suppressing VSD interaction and clustering actually enhanced channel activity as if the channels had nonetheless assembled in clusters.

This is shown in FIG. 3. In an embodiment, the surprisingly increased channel activity resulting from mutations in the N-termini is a consequence of impaired internal binding, suppressing the ‘locked-closed’ state, leading to enhanced K⁺ flux and stomatal kinetics.

As such, we have found that binding and clustering associate with, and impacts on the voltage- and K⁺-dependencies of GORK channel gating (Figure. 4). Most importantly, we show that this feature can be harnessed to accelerate stomatal kinetics and improve WUE without a cost in carbon assimilation. Modelling predicted and experiments demonstrated that manipulating GORK gating to enhance guard cell K⁺ flux accelerated stomatal kinetics as well as promoting stomatal conductance, gₛ (Figure. 5). These findings present a strategy, based on an ion channel native to the guard cell, for improving stomatal responsiveness and water use by the plant.

Accordingly, in one embodiment, the plant, part thereof or plant cell comprises at least one mutation in the cytosolic N termini of the channel, wherein the mutation prevents clustering of the channel. As shown in FIG. 1, the skilled person could, as an example, use the mating-based split ubiquitin screen (mbSUS), to determine whether a candidate mutation affects (i.e. prevents or reduces) clustering of the channel. As used herein, by “clustering” is meant the process by which individual potassium channels (each of which are made up of four subunits to form a tetramer) assemble or cluster together as a result of interactions between the VSDs at the periphery of the tetramer (FIG. 1a (1) and (2)).

In a further embodiment, the plant, part thereof or plant cell comprises at least one mutation in the cytosolic N termini whereby the mutation alters the pattern of charged amino acids in the cytosolic N termini. In one embodiment, “altering the pattern” means that where the cytosolic N termini comprises an alternating pattern of charged residues—that is, one or more positively charged residues followed by one or more negatively charged residues, or vice versa, with or without neutral amino acids interspersed in-between, followed by one or more positively charged residues and so on—this alternating pattern of residues is altered or changed by mutation such that the alternating pattern in part or in full is disrupted. For example—in a pattern of positive-negative-positive-negative-positive residues, as shown in FIG. 2, one or more of the charged residues/regions may be mutated to positive or negative residues.

As shown in FIG. 2, the wild-type cytosolic N termini of GORK comprises an alternating pattern of positive and negative amino acids. Mutation of the charged (positive) residues to negative residues (FIGS. 2b and c—gVSD^{PN}) or mutation of the charged (negative) residues to positive residues (FIGS. 2b and c—gVSD^{NP}) prevents clustering of the potassium channel.

Accordingly, in one embodiment, the plant, part thereof or plant cell comprises at least one mutation in the cytosolic N termini of the channel, wherein the mutation is the substitution of one or more negatively charged residues for positively charged residues or neutral residues. Preferably, enough residues are substituted to change the pattern of charged residues in the sequence. More preferably, all negatively charged residues are substituted for positively charged residues or neutral residues.

In another embodiment, the plant, part thereof or plant cell comprises at least one mutation in the cytosolic N termini of the channel, wherein the mutation is the substitution of one or more positively charged residues for negatively charged residues or neutral residues. Preferably, enough residues are substituted to change the pattern of charged residues in the sequence. More preferably, all negatively charged residues are substituted for positively charged residues or neutral residues.

Positively charged amino acids are Lysine (K), Arginine (R) and Histidine (H).

Negatively charged amino acids are: Aspartic Acid (D) and Glutamic acid (E).

Neutral amino acids are: Glycine (G), Alanine (A), Valine (V), Cysteine (C), Proline (P), Leucine (L), Isoleucine (I), Methionine (M), Tryptophan (W), Phenylalanine (F), Serine (S), Threonine (T), Tyrosine (Y), Asparagine (N) and Glutamine (Q).

However, as shown in FIG. 3, reversing the alternating pattern of charged residues (gVSD^{PNNP}) had no effect on clustering. Accordingly, the invention does not comprise a plant, part thereof or plant cell where the pattern of charged amino acid residues in the cytosolic N termini has been reversed. In one embodiment, therefore, the plant does not comprise a mutation in the cytosolic N termini where the mutation is the substitution of all positively charged residues for negatively charged residues and the substitution of all negatively charged residues for positively charged residues.

In another embodiment, the mutation is a deletion of one or more nucleotides in the cytosolic N termini. Preferably, the mutation is the deletion of all nucleotides in the cytosolic N termini.

The plant may be produced by any of the below-described methods. Preferably the mutation is introduced into at least one plant cell and a plant regenerated from the at least one mutated plant cell.

In an alternative aspect, the plant comprises and expresses at least one nucleic acid construct, wherein the nucleic acid construct comprises a nucleic acid sequence encoding a GORK channel, selected from SEQ ID NO: 26, 28 and 30 or a functional variant or homologue thereof. In one embodiment, the plant comprises and expresses at least one nucleic acid construct, wherein the nucleic acid construct comprises a nucleic acid sequence comprising a GORK nucleic acid sequence, selected from SEQ ID NO:25, 27 and 20 or a functional variant or homologue thereof. Preferably, the nucleic acid sequences are operably linked to a regulatory sequence, such as a constitutive, strong or tissue-specific (e.g. stomata-specific) promoter. The nucleic acid constructs may be introduced into a plant by transformation, as described in further detail below.

In another aspect of the invention, there is provided a method of increasing or accelerating stomatal conductance and/or the rate of stomatal opening and closing in a plant, the method comprising introducing at least at least of the above-described m above into a stomatal voltage-gated potassium channel. Enhancing the rate at which stomata open and close can promote photosynthetic carbon assimilation and water use efficiency (WUE). In one embodiment, the sensitivity of the channel to external potassium is increased at least one-fold, two-fold, three-fold, four-fold, five-fold or more compared to a wild-type or control channel.

In another aspect of the invention there is provided a method of increasing at least one of yield, water use efficiency (WUE) and/or carbon assimilation in a plant, the method comprising introducing at least at least one of the above-described mutations, into a stomatal voltage-gated potassium channel. In one embodiment, the method is an increase in WUE without a loss in C assimilation. As shown in FIG. 5, by mutating a GORK channel as described herein, the total dry biomass of the GORK^{kN-gS1-L4} and GORK^{NP} plants were enhanced 3.6±0.1- and 2.8±0.3-fold compared to controls under varying light, which translated to a highly significant improvement in WUE of 2.3±0.1 and 1.8±0.2-fold, respectively, when compared to the wild-type plants grown under the fixed light regime (FIG. 5f).

The term “yield” in general means a measurable produce of economic value, typically related to a specified crop, to an area, and to a period of time. Individual plant parts directly contribute to yield based on their number, size and/or weight. The actual yield is the yield per square meter for a crop per year, which is determined by dividing total production per year (includes both harvested and appraised production) by planted square metres.

In one embodiment, the increase in yield, is an increase in biomass or dry biomass. An increase in biomass can be measured by assessing any one or more of the following: (a) increased biomass (weight) of one or more parts of a plant, aboveground (harvestable parts), or increased root biomass, increased root volume, increased root length, increased root diameter or increased root length or increased biomass of any other harvestable part. Increased biomass may be expressed as g/plant or kg/hectare or (b) increased seed yield per plant, which may comprise one or more of an increase in seed biomass (weight) per plant or on an individual basis. All parameters are relative to a wild-type or control plant. The terms “seed” and “grain” as used herein can be used interchangeably. The terms “increase”, “improve” or “enhance” as used herein are also interchangeable. In one embodiment, an increase in yield comprises at least an increase in biomass, and in one embodiment, said increase in biomass is at least one-fold, two-fold, three-fold or four-fold or more compared to a wild-type or control plant.

By “water use efficiency” or “WUE” is meant the ratio of water used in plant metabolism to water lost through transpiration. In one example, WUE may be measured by measuring the amount of carbon fixed per water transpired. Alternatively, WUE may be measured by measuring the biomass per water transpired over a growing period or the amount of water fed to a plant. Other methods for measuring WUE would be well known to the skilled person.

By “carbon assimilation” or carbon fixation (such terms may be used interchangeably) is meant the conversion of inorganic carbon to organic compound, for example, through photosynthesis. In one example, carbon assimilation may be measured by measuring total biomass over time.

An “increase” as used herein, may refer to an increase of at least 1%, 2%, 3%, 4%, 5%, 6%, 7%, 8%, 9%, 10%, 20%, 30%, 40%, 50%, 60%, 70%, 80%, 90 or 95% or more compared to a control plant. Alternatively, the increase may at least a one-fold two-fold, three-fold, four-fold or five-fold increase, and preferably, between a one-fold and four-fold increase compared to a wild-type or control plant. The increases observed may be under light-fluctuating light conditions.

In an alternative embodiment, the method comprises introducing at least one mutation, as described above, into at least one, preferably endogenous, gene encoding a stomatal voltage-gated potassium channel.

By “at least one mutation” is means that where the stomatal voltage-gated potassium channel gene is present as more than one copy or homeologue (with the same or slightly different sequence) there is at least one mutation in at least one gene. Preferably all genes are mutated.

In one embodiment, the mutation is introduced using mutagenesis or targeted genome editing. That is, in one embodiment, the invention relates to a method and plant that has been generated by genetic engineering methods as described, and does not encompass naturally occurring varieties.

Targeted genome modification or targeted genome editing is a genome engineering technique that uses targeted DNA double-strand breaks (DSBs) to stimulate genome editing through homologous recombination (HR)-mediated recombination events. To achieve effective genome editing via introduction of site-specific DNA DSBs, four major classes of customisable DNA binding proteins can be used: meganucleases derived from microbial mobile genetic elements, ZF nucleases based on eukaryotic transcription factors, transcription activator-like effectors (TALEs) from Xanthomonas bacteria, and the RNA-guided DNA endonuclease Cas9 from the type II bacterial adaptive immune system CRISPR (clustered regularly interspaced short palindromic repeats). Meganuclease, ZF, and TALE proteins all recognize specific DNA sequences through protein-DNA interactions. Although meganucleases integrate nuclease and DNA-binding domains, ZF and TALE proteins consist of individual modules targeting 3 or 1 nucleotides (nt) of DNA, respectively. ZFs and TALEs can be assembled in desired combinations and attached to the nuclease domain of FokI to direct nucleolytic activity toward specific genomic Ioci.

A preferred genome editing method that can be used according to the various aspects of the invention is CRISPR. The use of this technology in genome editing is well described in the art, for example in U.S. Pat. No. 8,697,359 and references cited herein. In short, CRISPR is a microbial nuclease system involved in defense against invading phages and plasmids. CRISPR loci in microbial hosts contain a combination of CRISPR-associated (Cas) genes as well as non-coding RNA elements capable of programming the specificity of the CRISPR-mediated nucleic acid cleavage (sgRNA). Three types (1-Ill) of CRISPR systems have been identified across a wide range of bacterial hosts. One key feature of each CRISPR locus is the presence of an array of repetitive sequences (direct repeats) interspaced by short stretches of non-repetitive sequences (spacers). The non-coding CRISPR array is transcribed and cleaved within direct repeats into short crRNAs containing individual spacer sequences, which direct Cas nucleases to the target site (protospacer). The Type II CRISPR is one of the most well characterized systems and carries out targeted DNA double-strand break in four sequential steps. First, two non-coding RNA, the pre-crRNA array and tracrRNA, are transcribed from the CRISPR locus. Second, tracrRNA hybridizes to the repeat regions of the pre-crRNA and mediates the processing of pre-crRNA into mature crRNAs containing individual spacer sequences. Third, the mature crRNA:tracrRNA complex directs Cas9 to the target DNA via Watson-Crick base-pairing between the spacer on the crRNA and the protospacer on the target DNA next to the protospacer adjacent motif (PAM), an additional requirement for target recognition. Finally, Cas9 mediates cleavage of target DNA to create a double-stranded break within the protospacer.

One major advantage of the CRISPR-Cas9 system, as compared to conventional gene targeting and other programmable endonucleases is the ease of multiplexing, where multiple genes can be mutated simultaneously simply by using multiple sgRNAs each targeting a different gene. In addition, where two sgRNAs are used flanking a genomic region, the intervening section can be deleted or inverted (Wiles et al., 2015).

Cas9 is thus the hallmark protein of the type II CRISPR-Cas system, and is a large monomeric DNA nuclease guided to a DNA target sequence adjacent to the PAM (protospacer adjacent motif) sequence motif by a complex of two noncoding RNAs: CRISPR RNA (crRNA) and trans-activating crRNA (tracrRNA). The Cas9 protein contains two nuclease domains homologous to RuvC and HNH nucleases. The HNH nuclease domain cleaves the complementary DNA strand whereas the RuvC-like domain cleaves the non-complementary strand and, as a result, a blunt cut is introduced in the target DNA. Heterologous expression of Cas9 together with an sgRNA can introduce site-specific double strand breaks (DSBs) into genomic DNA of live cells from various organisms. For applications in eukaryotic organisms, codon optimized versions of Cas9, which is originally from the bacterium Streptococcus pyogenes, have been used.

The single guide RNA (sgRNA) is the second component of the CRISPR/Cas system that forms a complex with the Cas9 nuclease. sgRNA is a synthetic RNA chimera created by fusing crRNA with tracrRNA. The sgRNA guide sequence located at its 5′ end confers DNA target specificity. Therefore, by modifying the guide sequence, it is possible to create sgRNAs with different target specificities. The canonical length of the guide sequence is 20 bp. In plants, sgRNAs have been expressed using plant RNA polymerase Ill promoters, such as U6 and U3. Accordingly, using techniques known in the art it is possible to design sgRNA molecules that target the GORK sequence, and in particular, the cytosolic N-termini and/or the S2-S3 loop as described herein. In one embodiment, the sgRNA molecules target a sequence selected from SEQ ID No: 31, 32, 33, 34, 35, 36, 37, 38, 39, 40, 41, 42, 43 or a variant thereof as defined herein.

Cas9 expression plasmids for use in the methods of the invention can be constructed as described in the art.

In a preferred embodiment of any aspect of the invention described herein, sgRNA can be used with a modified Cas9 protein, such as nickase Cas9 or nCas9 or a “dead” Cas9 (dCas9) fused to a “Base Editor”—such as an enzyme, for example a deaminase such as cytidine deaminase, or TadA (tRNA adenosine deaminase) or ADAR or APOBEC. These enzymes are able to substitute one base for another. As a result no DNA is deleted, but a single substitution is made (Kim et al., 2017; Gaudelli et al. 2017). Alternatively, the method may use sgRNA together with a template or donor DNA construct, to introduce a targeted substitution, and in particular one of the substitutions described herein. In this embodiment, the introduction of a template DNA strand, following a sgRNA-mediated snip in the double-stranded DNA, can be used to produce a specific targeted mutation in the gene using homology directed repair. As a further alternative, prime editing can be used to introduce the specific mutation (Anzalone et al., 2019). Here a catalytically impaired Cas9 endonuclease is fused to an engineered reverse transcriptase programmed with a prime editing guide RNA (pegRNA) that is both specific to the target site and encodes the desired edit.

In an alternative embodiment, the nuclease used may be Cpf1 or MAD7.

Alternatively, more conventional mutagenesis methods can be used to introduce at least one mutation into stomatal voltage-gated potassium channel. These methods include both chemical and physical mutagenesis. Examples include T-DNA mutagenesis and targeting induced local lesions in genomes (TILLING). These techniques are well known in the art. Furthermore, rapid high-throughput screening procedures allow the analysis of amplification products for identifying a mutation in the potassium channel as described above, as compared to a corresponding non-mutagenised wild type plant. Once a mutation is identified in a gene of interest, the seeds of the M2 plant carrying that mutation are grown into adult M3 plants and screened for an alteration in stomatal kinetics.

Plants and plant parts obtained or obtainable by the methods described above which carry the above described nucleic acid constructs or carry a functional mutation in the potassium channel also within the scope of the invention.

In a further aspect of the invention there is provided a method of producing a plant with an increase in at least one of growth, yield, water use efficiency and/or carbon assimilation compared to a control plant, the method comprising introducing on or more of the above-described mutations into the plant.

Accordingly, in one embodiment, the method comprises
a. selecting a part of the plant;
b. transfecting at least one cell of the part of the plant of paragraph (a) with at least one sgRNA as described above;
c. regenerating at least one plant derived from the transfected cell or cells;
d. selecting one or more plants obtained according to paragraph (c) that have one or more mutations in the potassium channel, and preferably, in the cytosolic N termini of the channel.

Any of the genome editing constructs or nucleic acid constructs described herein, may be introduced into said plant through a process called transformation. The term “introduction” or “transformation” as referred to herein encompasses the transfer of an exogenous polynucleotide into a host cell, irrespective of the method used for transfer. Plant tissue capable of subsequent clonal propagation, whether by organogenesis or embryogenesis, may be transformed with a genetic construct of the present invention and a whole plant regenerated there from. The particular tissue chosen will vary depending on the clonal propagation systems available for, and best suited to, the particular species being transformed. Exemplary tissue targets include leaf disks, pollen, embryos, cotyledons, hypocotyls, megagametophytes, callus tissue, existing meristematic tissue (e.g., apical meristem, axillary buds, and root meristems), and induced meristem tissue (e.g., cotyledon meristem and hypocotyl meristem). The polynucleotide may be transiently or stably introduced into a host cell and may be maintained non-integrated, for example, as a plasmid. Alternatively, it may be integrated into the host genome. The resulting transformed plant cell may then be used to regenerate a transformed plant in a manner known to persons skilled in the art.

The transfer of foreign genes into the genome of a plant is called transformation. Transformation of plants is now a routine technique in many species. Advantageously, any of several transformation methods may be used to introduce the gene of interest into a suitable ancestor cell. The methods described for the transformation and regeneration of plants from plant tissues or plant cells may be utilized for transient or for stable transformation. Transformation methods include the use of liposomes, electroporation, chemicals that increase free DNA uptake, injection of the DNA directly into the plant, particle gun bombardment, transformation using viruses or pollen and microprojection. Methods may be selected from the calcium/polyethylene glycol method for protoplasts, electroporation of protoplasts, microinjection into plant material, DNA or RNA-coated particle bombardment, infection with (non-integrative) viruses and the like. Transgenic plants, including transgenic crop plants, are preferably produced via Agrobacterium tumefaciens mediated transformation. According to the invention, the nucleic acid is preferably stably integrated in the transgenic plants genome and the progeny of said plant therefore also comprises the transgene.

To select transformed plants, the plant material obtained in the transformation is, in certain embodiments, subjected to selective conditions so that transformed plants can be distinguished from untransformed plants. For example, the seeds obtained in the above-described manner can be planted and, after an initial growing period, subjected to a suitable selection by spraying. A further possibility is growing the seeds, if appropriate after sterilization, on agar plates using a suitable selection agent so that only the transformed seeds can grow into plants. Alternatively, the transformed plants are screened for the presence of a selectable marker such as the ones described above.

Following DNA or nucleic acid transfer and regeneration, putatively transformed plants may also be evaluated, for instance using Southern analysis, for the presence of one or more of the above-described mutations.

The generated transformed plants may be propagated by a variety of means, such as by clonal propagation or classical breeding techniques. For example, a first generation (or T₁) transformed plant may be selfed and homozygous second-generation (or T₂) transformants selected, and the T₂ plants may then further be propagated through classical breeding techniques. The generated transformed organisms may take a variety of forms. For example, they may be chimeras of transformed cells and non-transformed cells; clonal transformants (e.g., all cells transformed to contain the expression cassette); grafts of transformed and untransformed tissues (e.g., in plants, a transformed rootstock grafted to an untransformed scion).

In a further embodiment of any of the methods described herein, the method may further comprise at least one or more of the steps of assessing the phenotype of the genetically altered plant, measuring at least one of an increase in at least one of growth, yield, water use efficiency and/or carbon assimilation and comparing said phenotype to determine an increase in at least one of growth, yield, water use efficiency and/or carbon assimilation in a wild-type or control plant. In other words, the method may involve the step of screening the plants for the desired phenotype.

In a further aspect of the invention there is provided a plant obtained or obtainable by the above described methods.

The term “variant” or “functional variant” as used throughout with reference to any of the sequences described herein refers to a variant gene sequence or part of the gene sequence (such as a fragment) which retains the biological function of the full non-variant sequence. A functional variant also comprises a variant of the gene of interest, which has sequence alterations that do not affect function, for example in non-conserved residues. Also encompassed is a variant that is substantially identical, i.e. has only some sequence variations, for example in non-conserved residues, compared to the wild type sequences as shown herein and is biologically active. Alterations in a nucleic acid sequence that results in the production of a different amino acid at a given site that does not affect the functional properties of the encoded polypeptide are well known in the art. For example, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine, can also be expected to produce a functionally equivalent product. Nucleotide changes which result in alteration of the N-terminal and C-terminal portions of the polypeptide molecule would also not be expected to alter the activity of the polypeptide. Each of the proposed modifications is well within the routine skill in the art, as is determination of retention of biological activity of the encoded products.

As used in any aspect of the invention described throughout a “variant” or a “functional variant” has at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% overall sequence identity to the non-variant nucleic acid or amino acid sequence.

Two nucleic acid sequences or polypeptides are said to be “identical” if the sequence of nucleotides or amino acid residues, respectively, in the two sequences is the same when aligned for maximum correspondence as described below. The terms “identical” or percent “identity,” in the context of two or more nucleic acids or polypeptide sequences, refer to two or more sequences or subsequences that are the same or have a specified percentage of amino acid residues or nucleotides that are the same, when compared and aligned for maximum correspondence over a comparison window, as measured using one of the following sequence comparison algorithms or by manual alignment and visual inspection. When percentage of sequence identity is used in reference to proteins or peptides, it is recognised that residue positions that are not identical often differ by conservative amino acid substitutions, where amino acids residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Means for making this adjustment are well known to those of skill in the art. For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. Non-limiting examples of algorithms that are suitable for determining percent sequence identity and sequence similarity are the BLAST and BLAST 2.0 algorithms.

Suitable homologues can be identified by sequence comparisons and identifications of conserved domains. There are predictors in the art that can be used to identify such sequences. The function of the homologue can be identified as described herein and a skilled person would thus be able to confirm the function, for example when overexpressed in a plant.

Thus, the nucleotide sequences of the invention and described herein can also be used to isolate corresponding sequences from other organisms, particularly other plants, for example crop plants. In this manner, methods such as PCR, hybridization, and the like can be used to identify such sequences based on their sequence homology to the sequences described herein. Topology of the sequences and the characteristic domains structure can also be considered when identifying and isolating homologs. Sequences may be isolated based on their sequence identity to the entire sequence or to fragments thereof. In hybridization techniques, all or part of a known nucleotide sequence is used as a probe that selectively hybridizes to other corresponding nucleotide sequences present in a population of cloned genomic DNA fragments or cDNA fragments (i.e., genomic or cDNA libraries) from a chosen plant. The hybridization probes may be genomic DNA fragments, cDNA fragments, RNA fragments, or other oligonucleotides, and may be labelled with a detectable group, or any other detectable marker. Methods for preparation of probes for hybridization and for construction of cDNA and genomic libraries are generally known in the art and are disclosed in Sambrook, et al., (1989) Molecular Cloning: A Library Manual (2d ed., Cold Spring Harbor Laboratory Press, Plainview, New York).

Hybridization of such sequences may be carried out under stringent conditions. By “stringent conditions” or “stringent hybridization conditions” is intended conditions under which a probe will hybridize to its target sequence to a detectably greater degree than to other sequences (e.g., at least 2-fold over background). Stringent conditions are sequence dependent and will be different in different circumstances. By controlling the stringency of the hybridization and/or washing conditions, target sequences that are 100% complementary to the probe can be identified (homologous probing). Alternatively, stringency conditions can be adjusted to allow some mismatching in sequences so that lower degrees of similarity are detected (heterologous probing). Generally, a probe is less than about 1000 nucleotides in length, preferably less than 500 nucleotides in length. Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Duration of hybridization is generally less than about 24 hours, usually about 4 to 12. Stringent conditions may also be achieved with the addition of destabilizing agents such as formamide.

A plant according to all aspects of the invention described herein may be a monocot or a dicot plant. Preferably, the plant is a crop plant. By crop plant is meant any plant which is grown on a commercial scale for human or animal consumption or use. In a preferred embodiment, the plant is a cereal. In another embodiment the plant is Arabidopsis.

The plant according to the various aspects of the invention may be a moncot or a dicot plant. A dicot plant may be selected from the families including, but not limited to Asteraceae, Brassicaceae (eg Brassica napus), Chenopodiaceae, Cucurbitaceae, Leguminosae (Caesalpiniaceae, Aesalpiniaceae Mimosaceae, Papilionaceae or Fabaceae), Malvaceae, Rosaceae or Solanaceae. For example, the plant may be selected from lettuce, sunflower, Arabidopsis, broccoli, spinach, water melon, squash, cabbage, tomato, potato, yam, capsicum, tobacco, cotton, okra, apple, rose, strawberry, alfalfa, bean, soybean, field (fava) bean, pea, lentil, peanut, chickpea, apricots, pears, peach, grape vine or citrus species. In one embodiment, the plant is oilseed rape.

Also included are biofuel and bioenergy crops such as rape/canola, sugar cane, sweet sorghum, Panicum virgatum (switchgrass), linseed, lupin and willow, poplar, poplar hybrids, Miscanthus or gymnosperms, such as loblolly pine. Also included are crops for silage (maize), grazing or fodder (grasses, clover, sanfoin, alfalfa), fibres (e.g. cotton, flax), building materials (e.g. pine, oak), pulping (e.g. poplar), feeder stocks for the chemical industry (e.g. high erucic acid oil seed rape, linseed) and for amenity purposes (e.g. turf grasses for golf courses), ornamentals for public and private gardens (e.g. snapdragon, petunia, roses, geranium, Nicotiana sp.) and plants and cut flowers for the home (African violets, Begonias, chrysanthemums, geraniums, Coleus spider plants, Dracaena, rubber plant).

A monocot plant may, for example, be selected from the families Arecaceae, Amaryllidaceae or Poaceae. For example, the plant may be a cereal crop, such as wheat, rice, barley, maize, oat, sorghum, rye, millet, buckwheat, turf grass, Italian rye grass, sugarcane or Festuca species, or a crop such as onion, leek, yam or banana.

Preferably, the plant is a crop plant. By crop plant is meant any plant which is grown on a commercial scale for human or animal consumption or use. Preferred plants are maize, wheat, rice, oilseed rape, sorghum, soybean, potato, tomato, grape, barley, pea, bean, field bean, lettuce, cotton, sugar cane, sugar beet, broccoli or other vegetable brassicas or poplar.

In one embodiment, the plant is selected from Brassica, wheat, rice, oil palm, soybean, tomato, potato and pepper.

The term “plant” as used herein encompasses whole plants, ancestors and progeny of the plants and plant parts, including seeds, fruit, shoots, stems, leaves, roots (including tubers), flowers, tissues and organs, wherein each of the aforementioned comprise the nucleic acid construct as described herein or carry the herein described mutations. The term “plant” also encompasses plant cells, suspension cultures, callus tissue, embryos, meristematic regions, gametophytes, sporophytes, pollen and microspores, again wherein each of the aforementioned comprises the nucleic acid construct or mutations as described herein.

The invention also extends to harvestable parts of a plant of the invention as described herein, but not limited to seeds, leaves, fruits, flowers, stems, roots, rhizomes, tubers and bulbs. The aspects of the invention also extend to products derived, preferably directly derived, from a harvestable part of such a plant, such as dry pellets or powders, oil, fat and fatty acids, starch or proteins. Another product that may be derived from the harvestable parts of the plant of the invention is biodiesel. The invention also relates to food products and food supplements comprising the plant of the invention or parts thereof. In one embodiment, the food products may be animal feed. In another aspect of the invention, there is provided a product derived from a plant as described herein or from a part thereof.

In a most preferred embodiment, the plant part or harvestable part is a seed or the fruit. Therefore, in a further aspect of the invention, there is provided a seed or fruit produced from a genetically altered plant as described herein.

A control plant as used herein according to all of the aspects of the invention is a plant which has not been modified according to the methods of the invention. Accordingly, in one embodiment, the control plant does not have one or more of the above-described mutations. In one embodiment, the control plant is a wild type plant. The control plant is typically of the same plant species, preferably having the same genetic background as the modified plant.

While the foregoing disclosure provides a general description of the subject matter encompassed within the scope of the present invention, including methods, as well as the best mode thereof, of making and using this invention, the following examples are provided to further enable those skilled in the art to practice this invention and to provide a complete written description thereof. However, those skilled in the art will appreciate that the specifics of these examples should not be read as limiting on the invention, the scope of which should be apprehended from the claims and equivalents thereof appended to this disclosure. Various further aspects and embodiments of the present invention will be apparent to those skilled in the art in view of the present disclosure.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.

The foregoing application, and all documents and sequence accession numbers cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

The invention is now described in the following non-limiting examples.

### Example I: GORK Channels Interact Via the Voltage-Sensor Domain N-Terminus

We generated VSDs to test whether truncated channel domains would interact with the full-length channel protein, knowing that VSDs are expressed and function on their own in the membrane.

The yeast mating-based split-ubiquitin screen (mbSUS) yields growth of the diploid yeast on selective media only when the two halves of the split-ubiquitin moiety are brought together through interaction between putative protein interactors to which they are fused. mbSUS assays enable tests for interaction with full-length membrane proteins and the strength of interaction can be assessed by suppressing the bait protein fusion under the control of a Met-sensitive promoter. We used this screen to assess interactions of the full-length GORK as bait with the VSD of GORK (gVSD) as the prey. Strong growth was recovered on selective media, even in 500 μM Met, but growth was not recovered with the VSD of KAT1 (kVSD) or with full-length KAT1 as preys (FIG. 1b). These findings demonstrated an interaction with GORK that was specific to the structure of the gVSD.

### Example II: Interaction Depends on a Surface of Alternating Charged Residues

We carried out sequential domain substitutions in gVSD with the corresponding domains of kVSD in order to identify regions critical for gVSD interaction with GORK. Again, the VSD-GORK interactions were tested by mbSUS assay. Growth of the diploid yeast (FIG. 1c) in the presence of Met was recovered with chimeras that incorporated the cytosolic N-terminus of gVSD, even when the remaining structure was from kVSD. Conversely, interaction was strongly reduced when the N-terminus of gVSD was replaced with the kVSD N-terminus. Some growth was evident without added Met when yeast expressed the kVSD N-terminus in the GORK VSD backbone, specifically with the gVSD^{kN-gS1-L4} and gVSD^{kN-L1gS2-L4} preys. This weaker growth was lost when the N-terminus and the cytosolic S2-S3 loop of the gVSD backbone were both replaced with the corresponding kVSD sequences, suggesting the presence of an internal binding site for the VSD N-terminus of the full-length GORK, possibly in the GORK S2-S3 loop. We concluded that the primary site essential for binding was localized within the cytosolic N-terminus of 69 residues of the GORK channel.

Both the GORK and KAT1 N-termini harbor similarly charged amino acids in the first 21 residues, diverging thereafter before converging to a near-identical sequence near the base of the S1 α-helix. Deleting the first 23 residues of gVSD had no obvious effect on its interaction with GORK in mbSUS assays, but further truncations failed to recover yeast growth (FIG. 7) indicating that residues critical for interaction were situated among the subsequent 39 amino acids preceding the S1 α-helix. However, alanine-scanning mutagenesis of residues 23-66, individually and severally among the most divergent regions within this sequence failed to uncover a clear difference in yeast growth (FIG. 8), suggesting that interactions between the VSDs of GORK did not depend on a unique amino acid motif.

Aligning the N-termini of GORK and related channels of species from Brassica napus to wheat and rice showed a well-conserved pattern of alternating charges beyond the first 20 amino acids (FIG. 2a) that is not found in the KAT1 N-terminus (FIG. 2b). We reasoned that this alternation might present a charged surface ‘registered’ for a zipper-like interaction of the gVSD with a complementary surface on the full-length channel. Indeed, replacing all negatively-charged amino acids (Asp, Glu) with positively-charged residues (Lys) within these domains (gVSD^{NP}) suppressed diploid yeast growth in split-ubiquitin assays; growth was similarly suppressed when all positively-charged residues were replaced with negatively-charged residues (gVSD^{PN}) and when all of the charged residues were substituted with Ala. (gVSD^{A}). However, growth was recovered (FIG. 2c) when negatively- and positively-charged residues were exchanged (gVSD^{PNNP}), implying that the alternation in charged surfaces between the domains enables a registration of interactions with a complementarily charged surface to facilitate gVSD binding.

To support these findings, we carried out gel filtration analysis for N-terminal multimer assemblies in vitro and assessed VSD-GORK interaction by ratiometric bimolecular fluorescence complementation (rBiFC) in vivo. The in vitro approach offered quantitative information about possible multimer assemblies but was limited necessarily to work with the soluble N-termini. rBiFC analysis enabled a close comparison with the constructs expressed for the mbSUS studies and included internal controls for transformation and transgene loading. However, we did not expect the rBiFC studies to show puncta or a K+ dependency, because VSD binding would ‘cap’ the interacting surfaces of any GORK channel assembly (FIG. 1a) and BiFC annealing renders a largely stable complex. Gel filtration measurements showed that the gVSD and gVSD^{PN NP} N-termini eluted at apparent molecular weights near 26 kD, consistent with the formation of dimers as might be expected for inter-molecular binding between adjacent channels (FIG. 1a). By contrast, both the kVSD and gVSDNP N-termini eluted as monomers near 13 kD. Confocal imaging yielded similar results, showing a strong rBiFC signal in vivo on expressing GORK with gVSD and gVSD^{PNNP} but not with kVSD or the non-interacting mutants gVSD^{kN-gS1-L4} and gVSD^{NP}. Thus, the most parsimonious explanation is that an alternation in charged surfaces along the N-termini enables interactions, with a complementarily charged surface between the VSD N-termini of GORK.

### Example III: gVSD Interactions Affect GORK Clustering on Transient Expression

To assess clustering, initially we introduced a selection of these mutations in the full-length channel tagged with GFP and expressed the constructs transiently in tobacco leaves for confocal image analysis. After mounting, the upper epidermis of leaf segments was removed and the lower epidermis and remaining mesophyll were superfused with 0.1 mM K⁺ and then with 50 mM K⁺ while imaging. Channel clustering was quantified in the epidermal cells by measuring GFP fluorescence along the cell periphery and calculating the relative standard deviation (RSD) from each of six or more independent experiments.

As before, a pronounced redistribution of the punctate GFP fluorescence of the leaf tissue expressing GORK^{wt} when transferred from 0.1 mM to 50 mM K⁺. We observed (FIG. 9a) a similar K⁺ dependence to clustering for GORK^{PNNP} (SEQ ID NO: 23 and 24), but each of the mutants GORK^{kN-gS1-L4} (SEQ ID NO: 27 and 28), GORK^{PN} (SEQ ID NO: 29 and 30) and GORK^{NP} (SEQ ID NO: 25 and 26) gave little evidence of clustering, even in 0.1 mM K⁺. Analysis of these experiments (FIG. 9b) showed a significant decline in RSD with K⁺ elevations for both GORK^{wt} and GORK^{PNNP}; it also showed much reduced RSD values for GORK^{kN-gS1-L4}, GORK^{PN} and GORK^{NP} that were unaffected by K⁺ concentration, indicating that these mutant forms were dispersed around the cell periphery, their distribution independent of K⁺ outside. Thus, we conclude that the mutations eliminating gVSD binding with GORK also suppress GORK clustering.

GORK yields a current under voltage clamp that activates with positive-going voltage steps and decreasing K⁺ outside; as a consequence, the steady-state current-voltage (IV) curve shifts positive-going with increasing external K⁺. We observed much the same behaviour when GORK^{wt} was expressed in Xenopus oocytes (FIG. 10a). Expressing the same clustering-impaired constructs of GORK^{kN-gS1-L4}, GORK^{PN} and GORK^{NP} (FIG. 10b-d) led to negative-going shifts in the IV curves at each K⁺ concentration relative to GORK^{wt}, but not when positively- and negatively-charged residues were exchanged with the GORK^{PNNP} mutant (FIG. 10e).

The ensemble current of a voltage-sensitive ion channel can be described by a Boltzmann function of the form

$$I = {G}_{max} ( V - {E}_{K} ) / ( 1 + {e}^{δ F ( V - {V}_{1 / 2} ) / RT} ) \qquad [ 1 ]$$

where Gₘₐₓ is the maximum ensemble conductance, V is the voltage, E_{K} is the equilibrium voltage for K⁺, F is the Faraday constant, R is the universal gas constant, and T is the temperature. The parameters V_{1/2} and δ define the midpoint voltage for gating and the sensitivity of the gate to a change in voltage, respectively. To quantify gating, we resolved the ensemble parameters Gₘₐₓ, V_{1/2} and δ by joint, least-squares fitting to Eqn [1] of the steady-state K⁺ currents across the K⁺ concentrations. In each case, the analysis yielded visually satisfactory fittings with δ held in common and statistically significant shifts in V_{1/2} with K⁺ concentration. However, at any one K⁺ concentration, V_{1/2} values for GORK^{kN-gS1-L4}, GORK^{PN} and GORK^{NP} were displaced by −19±2 to −46±5 mV, consistent with a decrease in free energy for gating, ΔΔG, between −0.23 and −0.92 Kcal/mol. Indeed, plotting V_{1/2} as a function of K⁺ concentration showed that each of the mutants affected in clustering also retained a dependence on K⁺ outside; however, substantially higher K⁺ concentrations were required to achieve a V_{1/2} similar to the GORK^{wt}, indicating 3- to 8-fold decreases in the sensitivity to inhibition by K⁺ (FIG. 10f). In other words, mutating residues that promoted GORK clustering counterintuitively also appeared to reduce the free energy barrier for gating.

### Example IV: Manipulating GORK Alters Clustering and Enhances K⁺ Currents in Guard Cells

To assess the consequences of the GORK mutations in vivo, we generated stable transformants of GORK^{wt}, GORK^{kN-gS1-L4}, GORK^{NP} and GORK^{PNNP}, again expressing the proteins with GFP tags. Lines were generated under control of the constitutive pUB10 and guard cell-selective pGC1 promoters in the gork null mutant background of Arabidopsis. GORK transgene suppression had previously proven a difficulty, and we therefore analysed the T1 generation, after selecting plants for growth on Basta and GFP fluorescence, and T2 populations from at least three independent transformed lines of these T1 plants. In eight or more experiments with each construct, we observed no appreciable differences between transformants and the results were pooled accordingly. Plants complemented with GORK^{wt} and GORK^{PNNP} showed a peripheral clustering of the fluorescent marker in 0.1 mM K⁺ that dispersed when K⁺ outside was raised to 50 mM, much as observed in the wild type before. However, plants complemented with the non-interacting GORK mutations exhibited a diffuse distribution of GFP fluorescence around the guard cell periphery, even in 0.1 mM K⁺, that was unaffected by external K⁺ (FIG. 3).

We recorded GORK currents under voltage clamp from intact guard cells of the same plants and, as a control, from the gork null mutant background to confirm the lack of outward current. Recordings were carried out over an intermediate range of concentrations from 1 to 30 mM K⁺ (FIG. 4a-e) to expose shifts in K⁺-dependent gating. As in the oocytes, guard cells of wild-type Arabidopsis and the GORK^{wt} and GORK^{PNNP} complemented plants yielded currents that activated positive-going voltage steps and IV curves that were displaced positive-going with increasing external K⁺. Guard cells of plants complemented with the non-interacting GORK mutations showed similar currents; however, in this case the IV curves were shifted negative-going along the voltage axis at each K⁺ concentration compared to the wild type current and the GORK^{wt} transformants. Mean steady-state currents were well-fitted using the Boltzmann function of Eqn [1](FIG. 4a-e, solid lines) with a common value for δ but with significant displacements of V_{1/2} for the non-interacting mutants. At 30 mM K⁺, V_{1/2} values for GORK^{kN-gS1-L4} and GORK^{NP} were displaced by −28±4 and −17±3 mV (FIG. 4f) equivalent to ΔΔG values of −0.56 and −0.20 Kcal/mol and a decrease in sensitivity to K⁺ outside by factors of 7.4±0.9- and 4.5±0.4-fold, respectively, relative to the wild-type and GORK^{wt} transformants. Thus, as in oocytes, each of the non-interacting mutants reduced the inhibition of channel activity by external K⁺, whereas the GORK^{PNNP} complementation rescued the wild-type characteristics.

### Example VI: GORK Mutations Impact on Stomatal Dynamics and Gas Exchange

Because the non-interacting mutations enhanced GORK current with voltage and external K⁺ concentration, we anticipated that the effect should be evident as an acceleration in changes of stomatal conductance, gₛ, following a closing stimulus. Incorporating a −30 mV shift of V_{1/2} for GORK in simulations using the OnGuard3 platform predicted an acceleration in gₛ closing rates (FIG. 5a). Counterintuitively, they also predicted a small increase in the rate of opening, its dynamic range and steady-state gₛ that could be ascribed to an inward conductance and K⁺ uptake through GORK introduced by the shift in V_{1/2} (FIG. 4 and FIG. 11).

To test these predictions, we recorded the gas exchange characteristics of the same plants used for the analysis of clustering and channel gating. Experiments incorporated transitions to 200 μmol m⁻²s⁻¹ photosynthetically-active radiation (PAR) from the dark and back again after preconditioning with 200 μmol m⁻²s⁻¹ PAR. The results (FIG. 5a-c) confirmed a highly-significant accelerations of 1.4±0.1- and 1.6±0.2-fold for GORK^{NP} and GORK^{kN-gS1-L4}, respectively, in the rate of gₛ decline on transit to dark. They also showed a small acceleration of opening and increases of 1.1±0.1 and 1.2±0.1-fold in steady-state gₛ in the light, respectively.

One measure of plant performance is water use efficiency (WUE), often defined as the amount of dry mass produced per unit water transpired. WUE is affected by light through the combined influence on carbon demand and associated transpiration. With the enhanced gₛ and accelerated rates of stomatal movement we reasoned that the effects might translate to increases in WUE and biomass under fluctuating light. Light varies throughout the day in the natural environment, for example as clouds pass by. Photosynthesis generally tracks the energy input of light, but stomata are slower to respond to changes in light intensity, and this difference in kinetics can result in suboptimal assimilation rates when light intensity rises and to transpiration without corresponding assimilation when light intensity falls quickly. Thus, we reasoned that accelerating stomatal closure by manipulating GORK gating might enhance WUE and carbon assimilation when integrated over a period of vegetative growth under fluctuating light.

We compared the growth of wild-type plants, the gork null mutant and the gork lines complemented with GORK^{wt}, GORK^{kN-gS1-L4} and GORK^{NP} and GORK^{PNNP} under control of the pGC1 promoter. The complemented plants were selected as before. All lines were grown together under two light regimes, with plant exposed to a fixed daylight intensity of 140 μmol m⁻²s⁻¹ PAR or to the same daylight period with varying light and the equivalent total fluence. For the fluctuating light regime, we stepped the light intensity between 10 and 220 μmol m⁻²s⁻¹ with step intervals of 15 min, thus approximating the times normally required for stomatal closing and roughly half of that for opening. We also limited water availability under both regimes, maintaining soil moisture at 8±4% throughout the 5-week period of growth.

Rosette areas, fresh and dry weights of all plants were reduced under fluctuating light compared to the constant daylight conditions. Grown under the fluctuating light, however, the GORK^{kN-gS1-L4} and GORK^{NP} transgenic plants showed greater rosette areas and dry weights compared to the wild-type control, GORK^{wt} and GORK^{PNNP} lines, and only marginally reduced when compared to plants grown under the fixed light regime (FIG. 5d-f). Total dry biomass of the GORK^{kN-gS1-L4} and GORK^{NP} plants were enhanced 3.6±0.1 and 2.8±0.3-fold compared to the controls under varying light, which translated to a highly significant improvement in WUE of 2.3±0.1- and 1.8±0.2-fold, respectively, when compared to the wild-type plants grown under the fixed light regime (FIG. 5f). We confirmed that the increases in biomass were not the consequence of alterations in photosynthesis per se⁴²: CO₂ assimilation under saturating light (600 μmol m⁻² s⁻¹) was unaffected in any of the transgenic lines across the physiological range of internal CO₂ concentrations. Thus, complementing with GORK mutated to affect its gating, enhanced stomatal dynamics to improve water use efficiency and enhance carbon assimilation.

### Example VII: Methods

### Plant Growth, Transformation and Whole-Plant Physiology

Tobacco (Nicotiana tabacum) were grown and leaves transformed by Agrobacterium infiltration. Arabidopsis (Arabidopsis thaliana) wild-type (Col0), the gork null mutant and its complementation lines were sterilized and grown at 22:18° C. and 9:15 h light:dark cycles. The characteristics of the gork mutant background and its phenotype are described elsewhere.

Gas exchange measurements and growth experiments were carried out using LICOR 6800 gas exchange systems (Lincoln, USA) as described previously. Soil water content was monitored using a ML3 moisture sensor (DeltaT Devices, Cambridge UK) and plants watered at 1- to 3-d intervals to maintain 8±4% soil water content.

### Confocal Microscopy

Transformed tobacco and Arabidopsis leaves were imaged on a Leica SP8 SMD confocal microscope equipped with 20×/0.85 NA dry and 40×/1.3 NA oil lenses and hybrid GaAs detectors (Leica, Wetzlar, Germany). Fluorescence was excited with the 488 nm laser line and emitted light was collected across 495-550 nm for GFP. Chloroplast autofluorescence was collected across 630-690 nm. Laser intensities and detector gains were standardized between sets of experiments for quantitative analysis.

### Oocyte and Guard Cell Electrophysiology

Electrical recordings of K⁺ channels expressed in Xenopus oocytes were carried out using a standard protocols and a two-electrode voltage clamp. cRNAs were injected at 1 μg/oocyte. Recordings were carried out 48-72 h after injection and oocytes were collected and analyzed for protein expression. Oocytes were superfused with 1, 10, 30 and 60 mM K⁺ in modified ND96 buffer with 1.8 mM MgCl₂, 1.8 mM CaCl₂ and 10 mM HEPES-NaOH, pH 7.2 adjusted for osmotic balance^{31,66}. All recordings were carried out using Henry's EP suite (v. 3.5.5.5, Y-Science, Glasgow, UK).

Currents from intact guard cells in epidermal peels were recorded using double-barreled microelectrodes and Henry's EP suite. Guard cells were superfused with 1, 10, and 30 mM KCl in 5 mM Ca²⁺-MES, pH 6.1 ([Ca²⁺]=1 mM). Voltage was clamped in cycles from holding voltages at least −50 mV from E_{K} with positive-going steps for outward-rectifying currents. Voltage was clamped in cycles from a holding voltage of −100 mV with negative-going steps for inward-rectifying currents. Currents were analyzed using Henry's EP suite (Y-Science, Glasgow) and SigmaPlot 11.2 (Systat Software, Inc., USA) as described previously, and steady-state currents were fitted by joint, non-linear least-squares using the Boltzmann function of Eqn [1].

### OnGuard Modelling and Statistics

Quantitative modelling using OnGuard3. All other results are reported as means ±SE of n independent experiments. Significance was determined by Analysis of Variance (ANOVA), as appropriate with post-hoc analysis (Student-Newman-Keuls, Holm-Sidek and Tukey), and is indicated at P<0.05 unless otherwise stated.

### SEQUENCE LISTING

A. thaliana GORK CAC17380.1
SEQ ID NO: 1
MGRLRRRQEIIDHEEEESNDDVSSRRGKLSLAETFRWLDSSEHRRIETDGHNDYKYIIHPKNRWYKAWEMFI
LVWAIYSSLFTPMEFGFFRGLPERLFVLDIVGQIAFLVDIVLQFFVAYRDTQSYRTVYKPTRIAFRYLKSHFLMD
FIGCFPWDLIYKASGKHELVRYLLWIRLFRVRKVVEFFQRLEKDTRINYLFTRILKLLFVEVYCTHTAACIFYYLA
TTLPPEQEGYTWIGSLKLGDYSYENFREIDLWKRYTTALYFAIVTMATVGYGDIHAVNLREMIFVMIYVSFD
MVLGAYLIGNVTALIVKGSNTERFRDKMNDLISFMNRKKLGRDLRSQITGHVRLQYDSHYTDTVMLQDIPA
SIRAKIAQLLYLPYIKKVPLFKGCSTEFINQIVIRLHEEYFLPGEVITEQGNVVDHLYFVCEGLLEALVTKTDGSEE
SVTLLGPHTSFGDISIICNISQPFTVRVCELCHLLRLDKQSFSNILEIYFHDGRTILNNIMEEKESNDRIKKLESDIV
IHIGKQEAELALKVNSAAFQGDFYQLKSLIRSGADPNKTDYDGRSPLHLAACRGYEDITLFLIQEGVDVNLKD
KFGHTPLFEAVKAGQEGVIGLLVKEGASFNLEDSGNFLCTTVAKGDSDFLKRLLSSGMNPNSEDYDHRTPLH
VAASEGLFLMAKMLVEAGASVISKDRWGNSPLDEARLCGNKKLIKLLEDVKNAQSSIYPSSLRELQEERIERR
KCTVFPFHPQEAKEERSRKHGVVVWVPSNLEKLIVTAAKELGLSDGASFVLLSEDQGRITDIDMISDGHKLY
MISDTTDQT
>A. thaliana GORK mRNA
SEQ ID NO: 2
ATGGGACGTCTCCGGAGACGGCAAGAGATAATAGATCATGAAGAAGAGGAATCAAACGACGACGTTT
CATCAAGAAGAGGAAAACTCAGTTTAGCCGAGACGTTTCGGTGGCTTGATTCATCAGAGCATCGGAGA
ATTGAAACCGATGGTCATAATGATTATAAATACATCATTCATCCCAAAAACAGGTGGTACAAGGCATGG
GAAATGTTTATATTGGTGTGGGCAATATACTCCTCATTGTTCACTCCCATGGAGTTTGGTTTCTTCCGCG
GTCTGCCTGAGAGACTCTTTGTACTTGACATTGTTGGTCAGATCGCGTTCTTGGTCGATATTGTTCTTCA
GTTCTTTGTTGCCTATCGCGATACTCAGTCCTACCGGACTGTCTACAAACCAACACGTATTGCTTTCCGG
TACTTGAAGTCGCATTTTCTCATGGATTTCATCGGTTGCTTCCCTTGGGATCTTATTTATAAGGCATCAG
GGAAACATGAGTTGGTGAGGTACTTGTTGTGGATAAGGCTATTTCGGGTTCGCAAAGTGGTTGAGTTT
TTCCAAAGGCTTGAGAAAGACACAAGAATCAACTATCTATTCACTAGAATCTTAAAGCTCTTGTTCGTTG
AAGTTTATTGTACTCACACTGCTGCTTGTATCTTCTATTACTTGGCCACCACTCTTCCTCCAGAACAAGAA
GGGTACACGTGGATCGGTAGCTTGAAGCTAGGAGACTATAGCTACGAGAATTTCCGAGAAATCGATCT
ATGGAAACGTTATACTACTGCTCTATACTTCGCCATTGTCACTATGGCAACTGTCGGTTATGGAGACATT
CACGCGGTGAATCTGAGGGAAATGATATTTGTAATGATATATGTTTCATTTGATATGGTTCTCGGTGCTT
ACCTTATTGGTAACGTCACTGCCTTGATTGTGAAAGGTTCAAACACAGAGAGGTTCAGAGATAAAATGA
ATGATCTCATAAGTTTCATGAACCGCAAAAAACTCGGGAGAGACCTTCGTAGCCAGATAACTGGTCATG
TTAGATTGCAGTACGACAGTCACTACACCGACACTGTCATGCTTCAGGACATCCCAGCATCAATCCGCG
CCAAGATTGCGCAATTATTGTATCTGCCTTACATCAAAAAAGTTCCTCTCTTCAAAGGCTGCTCCACAGA
GTTTATCAATCAAATAGTTATAAGGCTCCATGAAGAGTATTTTCTTCCAGGAGAAGTAATAACAGAGCA
AGGAAACGTCGTGGATCATTTGTATTTCGTCTGTGAAGGCTTACTGGAGGCTCTTGTTACAAAAACAGA
TGGATCAGAAGAGAGTGTGACGTTACTTGGGCCTCACACTTCTTTTGGAGACATCTCCATCATTTGCAA
CATTTCTCAACCTTTCACTGTTAGGGTTTGTGAGCTATGCCATCTTTTACGACTCGATAAACAGTCTTTCT
CAAACATCCTCGAGATTTATTTTCACGACGGACGCACAATCTTGAACAATATTATGGAGGAGAAGGAAT
CAAATGATAGGATAAAGAAGCTAGAATCTGACATAGTGATTCACATTGGGAAACAAGAAGCAGAACTT
GCATTGAAAGTAAACAGTGCAGCTTTCCAAGGAGATTTTTACCAGCTTAAGAGCTTAATCCGATCTGGA
GCCGATCCTAACAAAACCGATTACGATGGAAGATCACCGCTTCATCTTGCAGCATGTAGAGGCTATGAA
GACATTACATTATTCCTTATTCAGGAAGGTGTTGATGTCAATCTAAAAGATAAGTTCGGACACACACCAT
TGTTTGAGGCTGTGAAAGCAGGACAAGAAGGAGTGATTGGTTTGCTTGTCAAAGAAGGAGCCTCCTTT
AATTTAGAAGATTCAGGAAACTTCCTTTGCACGACAGTTGCTAAAGGCGACTCTGATTTTCTCAAGAGA
TTGCTTTCAAGCGGTATGAACCCAAACAGTGAAGATTATGATCACAGAACGCCGCTTCATGTCGCGGCT
TCTGAAGGGTTATTCTTGATGGCTAAAATGTTGGTTGAAGCTGGAGCAAGCGTTATTTCTAAAGACCGA
TGGGGGAATTCTCCGCTTGATGAAGCCCGATTGTGCGGAAACAAGAAACTGATTAAGTTACTCGAAGA
TGTGAAAAATGCTCAGTCGTCTATCTACCCGTCAAGCTTGCGTGAATTACAAGAGGAGAGAATTGAGA
GACGGAAATGCACGGTGTTTCCATTCCACCCGCAAGAGGCGAAAGAAGAGCGTAGTAGAAAGCACGG
AGTTGTGGTTTGGGTCCCAAGCAATCTCGAGAAACTCATAGTAACCGCTGCGAAAGAGCTAGGGCTAT
CGGATGGAGCCTCATTTGTACTATTATCAGAAGACCAAGGTCGTATCACAGACATTGATATGATTAGTG
ATGGACACAAATTGTATATGATCAGTGATACTACTGATCAAACATAA
>Brassica napus GORK XP_013656069.1
SEQ ID NO: 3
MGCLRRRQESIAEEDDINDDVSRRRGGFSLAESFRWLDSPEHLKDDSDGPNEYPWIIKPSISRWYKAWELFI
LVWAIYSSLFTPMEFGFFRGLPENLFILDIVGQIAFLVDIVLQFFVAFQDKHNYRIDSKPTHIALRYLKSHFFLDL
VSCFPWDLIYKASGKHEVVRYILWIRLFRVRKVIEFFQRLEKDTRINYLFTRILKLIFVEVYCTHTAACIFYYLATT
LPAENEGYTWIGSLKLGDYSYENFRKIDIWKRYTTSLYFAIVTMATVGYGDIHAVNLREMIFVMIYVSFDMVL
GAYLIGNITALIVKGSNTERFRDKMNDLASFMNRKKLRGDIRSQITHHVRLQYDSKFTNTVMLQDIPASIRAK
IAQLLYTPYIEKIPLFKGCSSEFINQIVVRLHEEYFFPGEVITEQGNVVDHLYFVCEGSLEALETKTDGTEDLVELL
EPHTSFGDISIICNISQPFTIRVRSLCHLLRLDKQSFSNILEIYFHDGRKILNNLMEGKESNERIKKLESDIMIHIGK
QEAELALKVNSAAFQGDIYQLKSLVRSGADPNKTDYDGRAPLHLAASRGYEDITLFLIQEGVDINQKDKFGN
TPLLEAVKAGQDRVIDLLVKEGASFDLEDAGNFLCTVVVKGDSDFLKRLLSSGMDPNTEDYDHRTPLHVAAS
EGLFLMAKMLVEAGASVVAKDRWGNSPLDEARMCGNKKLIKLLEDADTAQPYIRPSSFHEPQDEKFERRKC
TVFPFHPHEEPSRKHGVMVWLPRDLQKLVETAAQELGISNEVSFVILAEEGGRITDIDMISDGQKLYLISDST
DQST
>Brassica napus GORK mRNA
SEQ ID NO: 4
ATGGGGTGTCTCCGGCGACGGCAAGAGAGTATAGCCGAGGAAGATGATATAAACGACGACGTTTCAA
GGAGAAGAGGTGGGTTCAGCTTGGCCGAGTCGTTCCGGTGGCTTGATTCCCCTGAGCATCTGAAAGAT
GATTCCGATGGCCCTAACGAATACCCTTGGATCATTAAGCCCTCAATTAGGTGGTACAAGGCATGGGA
GTTATTCATATTGGTCTGGGCAATATACTCGTCTTTGTTCACTCCCATGGAGTTTGGTTTCTTCCGCGGTC
TACCCGAAAACCTCTTCATACTCGACATTGTTGGTCAGATCGCATTTTTGGTCGATATTGTTCTACAGTTT
TTTGTCGCCTTCCAAGATAAGCATAACTACCGAATTGACAGCAAACCAACACATATTGCTCTCCGGTACT
TGAAATCGCATTTTTTCTTGGATTTGGTCAGTTGCTTCCCATGGGATCTCATCTATAAGGCTTCAGGGAA
ACATGAGGTGGTGAGGTACATATTGTGGATAAGGCTGTTTCGTGTGCGCAAAGTGATTGAGTTCTTCC
AAAGGCTTGAGAAAGACACGAGAATCAACTATCTCTTCACCAGAATCTTGAAGCTCATTTTCGTTGAGG
TTTACTGTACTCACACTGCTGCATGCATCTTCTATTACTTGGCCACCACTCTTCCTGCTGAGAACGAAGG
TTATACTTGGATCGGTAGCCTGAAGTTAGGAGACTATAGCTACGAGAATTTCCGAAAGATCGACATCTG
GAAACGTTACACCACATCTCTCTACTTCGCCATTGTCACTATGGCTACTGTCGGTTATGGAGACATACAT
GCCGTAAATTTAAGGGAAATGATATTTGTAATGATATATGTTTCGTTCGATATGGTTCTTGGTGCGTACC
TTATTGGTAACATCACTGCCTTGATTGTGAAAGGATCAAACACAGAGAGATTTAGAGATAAAATGAATG
ATCTGGCAAGTTTCATGAACCGAAAAAAACTTAGGGGAGACATTCGTAGCCAGATAACTCATCACGTTA
GATTGCAGTACGACAGTAAATTCACCAACACTGTCATGCTTCAAGACATCCCAGCCTCTATCCGCGCCA
AGATTGCGCAGTTATTATACACGCCTTACATTGAGAAAATCCCTCTGTTCAAGGGCTGCTCATCAGAGTT
TATTAACCAGATAGTTGTAAGGCTCCATGAAGAGTATTTTTTTCCAGGAGAAGTAATCACAGAGCAAGG
AAACGTCGTTGATCATTTGTATTTCGTCTGTGAAGGCTCACTGGAGGCTCTTGAAACAAAAACAGATGG
AACAGAAGACCTTGTGGAGTTGCTTGAGCCTCACACTTCTTTTGGTGATATATCCATCATCTGCAACATC
TCTCAACCTTTCACTATTAGAGTTCGTTCATTGTGCCATCTTTTACGCCTCGATAAACAGTCTTTCTCCAA
CATCCTCGAGATTTATTTTCATGATGGACGCAAAATCCTAAACAATCTTATGGAGGGTAAAGAATCAAA
TGAGAGGATAAAGAAGCTCGAATCTGACATTATGATTCACATTGGGAAACAAGAAGCAGAACTTGCAT
TGAAGGTAAACAGTGCAGCTTTCCAAGGAGATATCTACCAGCTTAAAAGCTTAGTCCGCTCTGGCGCCG
ATCCTAACAAAACAGATTACGATGGAAGAGCACCGCTTCATCTTGCAGCGTCTAGGGGATACGAAGAC
ATTACATTGTTTCTTATTCAAGAAGGCGTCGATATAAATCAAAAAGATAAATTTGGCAATACACCATTGT
TAGAGGCTGTAAAAGCAGGGCAAGACAGAGTGATCGATTTGCTTGTCAAAGAAGGAGCTTCCTTTGAT
TTAGAAGATGCTGGAAACTTTCTCTGCACGGTAGTTGTGAAAGGCGACTCTGATTTTCTTAAGAGATTG
CTCTCAAGCGGTATGGACCCAAACACCGAAGATTATGATCATAGAACACCGCTTCATGTCGCTGCTTCA
GAAGGCTTATTCTTGATGGCTAAGATGCTTGTTGAAGCTGGAGCTAGCGTTGTCGCTAAAGACCGGTG
GGGAAACTCTCCGCTTGATGAAGCTCGAATGTGTGGAAACAAGAAATTGATTAAGTTACTTGAAGACG
CGGATACAGCTCAGCCTTATATACGCCCGTCAAGCTTTCATGAACCACAAGATGAGAAATTTGAGAGAC
GGAAATGCACAGTGTTTCCATTCCACCCACATGAGGAGCCGAGTAGAAAGCATGGAGTTATGGTTTGG
CTTCCGCGTGACCTCCAGAAACTTGTGGAAACAGCTGCGCAAGAGCTCGGGATATCTAATGAAGTCTCC
TTTGTAATATTGGCCGAGGAAGGAGGTCGAATCACAGACATTGATATGATCAGTGATGGACAGAAACT
GTATTTGATCAGTGATAGTACTGATCAATCAACCTGA
>Raphanus sativus (Radish) GORK XP_018433301.1
SEQ ID NO: 5
MGCLRRPQESIAEEDDTNDDVSMRRGRFSLAESFRWLDSPEHLKNDSDCPNEHPWIINPSNRWYKAWELF
ILLWAIYSSLFTPMEFGFFRGLPENLFVLDIIGQIAFLVDIVIQFFVAFQDKHTYRTDDKPTHIALRYMKSHFFL
DLVSCFPWDLIYKASGKNEVVRYILWIRLFRVRKVIEFFQRLEKDTRINYLFTRILKLIFVEVYCTHTAACIFYYLA
TTLPAENEGYTWIGSLKLGDYSYENFRKIDIWKRYTTSLYFAIVTMATVGYGDIHAVNLREMIFVMIYVSLDM
VLGAYLIGNITALIVKGSNTERFRDKMNDLASFMNRKKLRGDIRSQITHHVRLQYDSKFTNTVMLQDIPASIR
AKIAQLLYTPYIEKIPLFKGCSSEFINQIVIRLHEEYFFPGEVITEQGNVVDHLYFVCEGSLEALATKTDGTEELVA
LLEPHTSFGDISIICNISQPFTVRVRSLCHLLRLDKQSFSNILEIYFHDGRKILNNLMEGKESNERIKELEPDVMIH
IGKQEAELALKVNSAAFQGDIYQLKRLVRSGADPNKTDYDGRAPLHLAASRGYEDITLFLIQEGVDINQKDKF
GNTPLLEAVKAGQDRVIHVLVKEGASFDLEDAGNFLCTVVLKGDSDFLRRLLSSGMDPNTEDYDHRTPLHV
AASEGLFLMAKMLVEAGARVVAKDRWGNSPLDEARICGNKKLIKLLENARTAQPSICPSSSHELQDEKFKRR
KCTVFPFHPHEEPNRKHGVMVWLPRDLQKLVETAAQELRLSNEASFVILSEEGGRITDVDMISDGQKLYLIS
DSTDQSA
>Raphanus sativus (Radish) GORK mRNA
SEQ ID NO: 6
ATGGGGTGTCTCCGGCGACCGCAAGAGAGTATAGCCGAGGAAGACGATACAAACGACGACGTTTCAA
TGAGAAGAGGTAGATTCAGCTTGGCCGAGTCGTTCCGGTGGCTTGATTCCCCCGAGCATCTGAAAAAT
GATTCCGATTGCCCTAACGAACACCCTTGGATCATTAATCCCTCAAACAGGTGGTACAAGGCATGGGAG
TTATTCATATTGTTATGGGCAATATATTCGTCTTTGTTCACTCCCATGGAGTTTGGTTTCTTCCGCGGTTT
ACCCGAAAACCTCTTCGTACTCGACATTATTGGTCAGATTGCATTTTTGGTCGATATTGTTATACAGTTTT
TCGTTGCCTTCCAAGATAAACATACCTACCGGACTGACGACAAACCAACACATATTGCTCTCCGGTACAT
GAAATCGCATTTTTTCTTGGATTTGGTCAGTTGCTTCCCATGGGATCTCATCTATAAGGCGTCAGGGAAA
AACGAGGTGGTGAGGTACATATTGTGGATAAGGCTGTTTCGTGTGCGCAAAGTGATAGAGTTCTTCCA
AAGGCTTGAGAAAGACACGAGAATCAACTATCTGTTCACCAGAATCTTGAAGCTCATTTTTGTTGAGGT
TTATTGTACTCACACTGCTGCATGCATCTTCTATTACTTGGCCACCACTCTTCCTGCCGAGAACGAAGGT
TACACTTGGATCGGTAGCCTGAAGTTAGGAGACTATAGCTACGAGAATTTCCGAAAGATCGACATTTG
GAAACGTTATACCACATCTCTCTACTTCGCCATTGTCACTATGGCTACTGTCGGTTATGGAGACATACAC
GCGGTAAATTTAAGGGAAATGATATTCGTAATGATATATGTTTCACTCGATATGGTTCTGGGTGCGTAC
CTTATTGGTAACATCACTGCCTTGATAGTGAAAGGATCAAACACAGAGAGATTTAGAGATAAAATGAAT
GATCTGGCAAGTTTCATGAACCGAAAAAAACTACGGGGAGACATTCGAAGCCAGATAACTCATCACGT
TAGATTGCAATACGACAGTAAATTCACCAACACTGTCATGCTTCAAGACATCCCAGCCTCTATCCGCGCC
AAGATTGCCCAATTATTATACACGCCTTACATTGAGAAAATCCCTCTGTTCAAGGGCTGTTCATCAGAGT
TTATTAACCAGATAGTTATAAGGCTCCACGAAGAGTATTTTTTTCCGGGAGAAGTAATCACAGAGCAAG
GAAACGTTGTTGATCATTTGTATTTCGTCTGTGAAGGCTCACTGGAGGCTCTTGCTACAAAAACAGATG
GAACAGAAGAGCTTGTGGCGTTGCTTGAGCCTCACACTTCTTTTGGTGATATCTCCATCATTTGCAACAT
CTCTCAACCTTTCACTGTTAGAGTTCGTTCATTATGCCATCTTTTACGCCTGGATAAACAGTCTTTCTCCA
ACATCCTCGAGATTTATTTTCATGATGGACGCAAAATTCTAAACAATCTTATGGAGGGTAAGGAATCAA
ATGAGAGGATAAAGGAGCTAGAACCTGATGTTATGATTCACATTGGGAAACAAGAAGCAGAACTTGCA
CTGAAGGTAAACAGTGCAGCTTTCCAAGGAGATATCTACCAGCTTAAAAGATTAGTCCGCTCTGGAGCC
GATCCTAACAAAACAGATTACGATGGAAGAGCACCGCTTCATCTTGCAGCGTCTAGGGGATACGAAGA
CATTACATTGTTTCTTATTCAAGAAGGCGTCGATATAAATCAAAAAGATAAATTTGGGAATACACCATTG
TTAGAGGCTGTAAAAGCAGGGCAAGACAGAGTGATCCATGTGCTTGTCAAAGAAGGAGCTTCCTTTGA
TTTAGAAGATGCTGGAAACTTTCTTTGCACGGTCGTTCTGAAAGGCGACTCTGATTTTCTTAGGAGATT
GCTCTCAAGCGGTATGGACCCAAACACCGAAGATTATGATCATAGAACACCACTTCATGTCGCTGCTTC
TGAAGGATTATTCTTGATGGCTAAGATGCTTGTTGAAGCTGGAGCTAGAGTTGTCGCTAAAGACAGGT
GGGGAAACTCTCCTCTTGATGAAGCTCGAATATGTGGAAACAAGAAATTGATTAAGTTACTTGAAAAC
GCGAGAACAGCTCAGCCTTCTATATGCCCGTCAAGCTCTCATGAACTACAAGATGAGAAATTTAAGAGA
CGGAAATGCACAGTGTTTCCATTCCACCCACACGAGGAGCCTAATAGAAAGCATGGAGTCATGGTGTG
GCTTCCGCGTGATCTACAGAAACTTGTGGAAACAGCTGCGCAAGAGCTCAGGCTATCTAATGAAGCCTC
CTTTGTAATATTGTCAGAGGAAGGAGGTCGAATCACAGACGTTGATATGATCAGTGATGGACAGAAAC
TGTATTTGATCAGTGATAGTACTGATCAATCAGCGTGA
>Elaeis guineensis(Oil palm) KOR1 XP_010905454.2
SEQ ID NO: 7
MSPRAKNGSPEIEYELEEPIASSRGIRLFLLTSEFALGPLRRRRATSQEKLLERFVIEPDNRWYQLWTRFILVWA
LYSSFFTPIEFGFFRGLPNNLFWLDFAGQVAFLVDIFVQFLVAYRDSHTYRMIYEPTSIAVRYAKSSFVFDLLGC
FPWDAIYRACGRKEEVRYLLWIRLTRVRKVTDFFQKMEKDIRINYLFTRIVKLIVVELYCTHTAACIFYYLATTLP
ASMEGYTWIGSLKLGDYSYSHFRDMDIAKRYITSLYFAIVTMATVGYGDIHAVNPREMVFIMIYVSFDMILG
AYLIGNMTALIVKGSKTERFRDRMKELIKYMNRNKLGKDIRDQIKGHVRLQYESSYTEASVLQDIPVSIRAKIS
QTLYKPYIENVPLFKGCSAEFIHQIIIKLQEEFFLPGEVILEQGNAVDQLYFVCHGKLEGVGIGEDGPLELEPNSS
FGEIAILCNIPQPYTVRVCELCRLLRIDKQIFTNILQIYFVDGRKILSNLLEGNDSIQIKQLEGDIIFHIGKQEAELA
LRVNNAAFYGDLHYLKGLIQAGADPNKTDYDGRSPLHLAAAKGHEDITLFLIQEGVNINLSDNFGNTPLFEA
VKVGHDRVASLLFSRGAQLNLKDAGSHLCTAAAKGDSDFIRRALSYGVDPNSKDYDHRTALHIAASEGLYFI
AKLLLEAGASVFAMDRWGATPLDEGRKSGNQSLMLLLEGAKSDELSKFPEHAREVQDKMHPRRCTVFPFH
PWDPKEARREGVVLWIPCTIEELIRSSREQLKSSGSCILSEDGGRILDVDMISDGQKLYLVADQETTAHNNQH
T
>Elaeis guineensis(Oil palm) KOR1 mRNA
SEQ ID NO: 8
ATGGGTCCCCGGAGATCGAGTACGAGTTGGAGGAGCCGATTGCGTCGTCGCGGGGGATCCGCCTTTTC
CTGCTCACCTCCGAATTCGCCCTCGGCCCCCTCCGCCGCCGCCGGGCGACCAGCCAGGAGAAGCTTCTC
GAGCGCTTCGTCATCGAACCCGATAACCGGTGGTACCAGCTGTGGACGAGGTTTATACTGGTATGGGC
ATTGTACTCGTCCTTCTTCACGCCGATAGAGTTCGGCTTCTTCAGGGGGCTCCCGAACAACCTCTTCTGG
CTCGATTTCGCCGGCCAGGTGGCCTTCCTCGTCGACATCTTCGTCCAGTTCCTCGTCGCCTACCGTGACT
CTCACACCTACCGCATGATCTACGAGCCCACCTCCATTGCTGTCCGGTACGCGAAGTCGAGCTTCGTCTT
CGACTTACTTGGCTGCTTTCCTTGGGATGCTATCTATAGGGCATGCGGACGTAAAGAGGAAGTGAGGT
ACCTTTTATGGATTCGATTAACTCGGGTGCGCAAAGTCACAGATTTTTTTCAGAAGATGGAGAAAGATA
TCCGCATCAACTACTTGTTTACAAGAATTGTTAAACTCATAGTTGTGGAGCTCTACTGCACGCATACAGC
TGCCTGTATATTTTACTATCTTGCAACAACTTTGCCAGCATCCATGGAAGGCTATACATGGATAGGGAG
CTTAAAGTTGGGTGATTATAGCTATTCGCACTTCAGGGATATGGATATCGCAAAGCGTTACATAACATC
GTTATATTTTGCCATTGTCACAATGGCAACTGTTGGTTATGGAGACATACATGCAGTTAATCCCAGGGA
GATGGTATTCATCATGATTTATGTTTCTTTTGATATGATTCTGGGAGCTTACCTTATTGGCAACATGACT
GCATTGATTGTGAAAGGATCAAAGACAGAAAGGTTTAGGGACAGAATGAAAGAATTAATTAAATACAT
GAACAGAAACAAACTTGGAAAGGATATTCGTGACCAGATCAAAGGACATGTACGCTTACAATATGAAA
GCAGCTACACAGAGGCTTCAGTTCTTCAGGATATCCCAGTTTCAATTCGTGCTAAGATATCACAGACACT
ATACAAACCGTATATCGAAAATGTTCCCCTGTTTAAGGGATGCTCAGCCGAATTCATTCACCAGATTATT
ATCAAGCTGCAGGAAGAGTTTTTCCTTCCAGGGGAGGTGATATTGGAACAGGGAAATGCAGTAGATCA
GCTATATTTTGTCTGCCATGGTAAGCTGGAAGGAGTAGGAATTGGTGAAGATGGACCTTTGGAACTGG
AGCCCAACAGTTCCTTTGGAGAAATTGCGATTCTGTGCAACATTCCTCAGCCTTACACTGTTCGTGTTTG
TGAATTGTGTAGACTTTTGCGAATTGATAAACAAATCTTCACAAATATTCTACAAATTTACTTTGTTGATG
GTCGGAAAATTTTGAGCAACCTTCTAGAGGGTAATGATTCCATTCAAATTAAGCAACTAGAAGGTGATA
TTATATTCCATATCGGTAAGCAAGAAGCCGAGCTTGCTTTAAGAGTAAACAATGCTGCTTTCTATGGAG
ATCTCCATTACCTTAAAGGTTTGATCCAAGCAGGAGCAGATCCTAATAAGACTGATTATGATGGACGCT
CACCTTTGCATCTCGCAGCAGCAAAAGGGCATGAAGATATCACTCTATTTCTCATTCAAGAAGGCGTGA
ACATTAACCTTTCTGACAACTTTGGAAATACACCCTTGTTTGAGGCAGTCAAGGTTGGACATGATCGAG
TGGCTTCTTTACTCTTTAGTAGAGGTGCACAATTAAATCTTAAAGATGCTGGTAGCCACCTTTGTACAGC
TGCTGCAAAGGGGGATTCAGATTTTATTAGAAGAGCTTTATCTTACGGTGTTGATCCAAACTCAAAGGA
CTACGATCACCGCACAGCGCTGCATATAGCTGCTTCTGAGGGATTATATTTTATAGCAAAGTTACTITTA
GAGGCTGGGGCAAGTGTTTTTGCTATGGACAGATGGGGAGCTACACCATTGGATGAAGGACGAAAGA
GTGGAAACCAATCCTTGATGCTGCTGTTAGAAGGTGCAAAGTCTGACGAGCTGTCCAAGTTTCCTGAAC
ATGCTCGGGAAGTTCAAGATAAAATGCATCCAAGGAGGTGCACAGTGTTTCCTTTTCACCCGTGGGATC
CGAAAGAAGCAAGAAGAGAGGGAGTCGTGCTATGGATCCCATGCACCATAGAGGAGCTCATTAGATC
CTCTAGAGAGCAGCTAAAGTCTTCAGGTTCATGCATACTATCAGAAGATGGGGGCAGGATTCTTGATGT
AGATATGATATCTGATGGCCAGAAATTATATCTAGTTGCAGATCAAGAAACAACTGCACATAATAACCA
GCATACTTGA
>O. sativa KOR1 (rice)XP_015644419.1
SEQ ID NO: 9
MGRGIGSKRRVEDDDGENMPGRKKKEEEEEEEDDDGEEEYEVDVVRDRIGSSRGSRLALFGSDLRLGRFRP
RRRRVAPVDGDDGIFQDFVIDPDNKWYRLWTRFILVWAVYSSFFTPLEFGFFRGLPRNLFFLDIAGQIAFLIDI
VLRFFVAYRDPDTYRMVHNPTSIALRYCKSSFIFDLLGCFPWDAIYKACGSKEEVRYLLWIRLTRAMKVTEFFR
SMEKDIRINYLFTRIVKLIVVELYCTHTAACIFYYLATTLPESMEGYTWIGSLOLGDYSYSHFREIDLTKRYMTSL
YFAIVTMATVGYGDIHAVNVREMIFIMIYVSFDMILGAYLIGNMTALIVKGSRTERFRDKMKEVIRYMNRNK
LGKDIREQIKGHLRLQYESSYTEASVLQDIPVSIRAKISQTLYKPYIESIPLFKGCSAEFIQQIVIRLQEEFFLPGEVI
LEQGSAVDQLYFVCHGALEGVGIGEDGQEETILMLEPESSFGEIAVLCNIPQPFTVRVCELCRLLRLDKQSFTN
ILEIFFVDGRRILSNLSESSEYGSRIKQLESDITFHIGKQEAELTLRVNNAAFYGDMHQLKSLIRAGADPKNTDY
DGRSPLHLAACKGFEDVVQFLLHEGVDIDLSDKFGNTPLLEAVKQGHDRVATLLFSKGAKLSLENAGSHLCT
AVARGDTDFVRRALAYGGDPNARDYDHRAPLHIAAAEGLYLMAKLLVDAGASVFATDRWGTTPLDEGRR
CGSRTMVQLLEAAKSGELSRYPERGEEVRDKMHPRRCSVFPHHPWDGGERRREGVVVWIPHTIEGLVSSA
QEKLGLAGSGEGLRLLGEDGARVLDVDMVHDGQKLYLVVGGGGDDGGTEARQ
>O. sativa KOR1 (rice)
SEQ ID NO: 10
ATGGGGAGGGGGATAGGGTCGAAGCGGAGGGTGGAGGACGACGACGGGGAGAATATGCCGGGGAG
GAAGAAGAAGGAGGAGGAGGAGGAGGAGGAGGATGACGATGGGGAAGAGGAGTACGAGGTGGAC
GTGGTGCGCGACCGGATCGGGTCGTCGCGGGGGAGCCGCCTGGCGCTGTTCGGCTCCGACCTCCGCCT
CGGCCGCTTCCGCCCGCGCCGCCGCCGCGTCGCCCCCGTCGACGGCGACGATGGCATCTTCCAGGACT
TCGTCATCGACCCCGACAACAAGTGGTATCGGTTATGGACGAGGTTCATACTAGTGTGGGCAGTGTAC
AGTTCCTTCTTCACGCCATTGGAATTTGGCTTCTTCAGGGGGCTCCCCAGGAACCTCTTCTTCCTAGACA
TAGCTGGTCAGATCGCCTTCCTCATCGACATTGTTCTCAGGTTCTTCGTGGCCTACCGTGATCCTGACAC
CTACCGCATGGTTCACAATCCCACCTCCATCGCTCTCCGATATTGTAAATCAAGCTTCATTTTTGATCTTC
TTGGTTGTTTTCCGTGGGATGCTATCTATAAGGCTTGTGGAAGTAAAGAAGAAGTAAGATACCTATTGT
GGATTCGGTTGACACGAGCTATGAAGGTGACAGAGTTCTTCAGGAGCATGGAAAAAGATATCCGTATA
AATTACCTGTTTACTAGGATTGTGAAACTCATAGTTGTGGAACTCTACTGTACGCACACAGCAGCATGT
ATCTTCTACTATCTTGCAACAACACTTCCTGAATCAATGGAAGGATATACATGGATAGGGAGCTTGCAG
TTGGGAGACTATAGTTATTCTCATTTCAGGGAGATTGATCTTACCAAGCGTTATATGACATCGCTGTACT
TTGCCATAGTCACCATGGCAACTGTTGGTTATGGTGACATTCATGCTGTGAATGTCAGGGAGATGATAT
TCATCATGATTTATGTTTCCTTTGATATGATTCTTGGAGCCTACCTCATTGGTAACATGACTGCACTTATT
GTCAAGGGCTCGAGAACCGAGCGATTCAGGGATAAGATGAAAGAAGTTATCAGGTACATGAACAGAA
ACAAGCTTGGAAAGGACATAAGGGAACAGATTAAAGGACATTTGAGGTTGCAGTATGAAAGCAGCTA
CACTGAAGCTTCTGTCCTTCAGGATATCCCAGTCTCAATTCGTGCGAAGATTTCACAAACACTGTATAAG
CCATATATCGAAAGCATTCCACTGTTTAAAGGATGTTCAGCAGAATTCATTCAACAGATTGTGATCAGG
CTGCAGGAAGAGTTCTTCCTACCCGGAGAGGTTATTTTGGAGCAGGGAAGTGCAGTTGATCAACTGTA
CTTTGTCTGTCATGGGGCACTGGAAGGTGTTGGCATTGGTGAAGATGGTCAAGAAGAGACCATTCTAA
TGCTAGAGCCTGAAAGTTCTTTTGGAGAAATCGCTGTTCTCTGCAATATCCCACAACCCTTCACCGTTCG
TGTCTGCGAACTTTGTCGTCTTTTGCGGCTTGACAAGCAATCCTTCACAAACATCCTGGAGATCTTCTTC
GTTGATGGAAGAAGAATTCTGAGCAACCTTTCTGAGAGTAGCGAATACGGCAGCCGGATCAAGCAGCT
GGAATCCGACATCACGTTCCACATCGGGAAGCAAGAGGCAGAGCTGACGCTGCGAGTGAACAACGCT
GCGTTCTACGGTGACATGCATCAGCTCAAGAGCCTGATCCGTGCAGGAGCCGATCCCAAGAACACCGA
TTACGACGGGCGATCTCCTCTGCATCTTGCGGCTTGCAAAGGGTTCGAAGATGTCGTCCAGTTCCTCCT
CCACGAAGGCGTCGACATCGATCTTTCCGACAAATTTGGCAACACGCCATTGCTGGAGGCGGTGAAGC
AGGGGCACGACCGGGTGGCGACGCTGCTGTTCAGCAAGGGGGCGAAGCTGAGCCTGGAGAACGCCG
GGAGCCACCTGTGCACGGCGGTGGCGAGGGGCGACACGGACTTCGTCCGGCGGGCGCTCGCCTACGG
CGGCGACCCGAACGCGAGGGACTACGACCACCGCGCGCCGCTCCACATCGCCGCCGCCGAGGGCCTCT
ACCTGATGGCGAAGCTGCTCGTCGACGCCGGCGCCAGCGTGTTCGCCACCGACCGGTGGGGCACGAC
GCCGCTGGACGAAGGGCGCAGGTGCGGGAGCCGGACGATGGTGCAGCTGCTGGAGGCGGCCAAGTC
CGGCGAGCTCTCCAGGTACCCGGAGCGCGGCGAGGAGGTGCGGGACAAGATGCACCCGCGGCGGTG
CTCCGTGTTCCCGCACCACCCGTGGGACGGCGGCGAGCGGCGGCGGGAAGGGGTGGTGGTGTGGATC
CCGCACACCATCGAGGGGCTCGTCAGCTCGGCGCAGGAGAAGCTCGGCCTCGCCGGCTCCGGCGAGG
GGCTCCGGCTGCTCGGCGAGGACGGCGCCAGAGTGCTCGACGTCGACATGGTCCACGACGGGCAGAA
GCTCTACCTCGTCGTCGGCGGCGGCGGCGATGACGGCGGCACGGAGGCAAGGCAGTGA
SEQ ID NO: 11: >A. thaliana SKOR NM_111153.4
MGGSSGGGVSYRSGGESDVELEDYEVDDFRDGIVESRGNRFNPLTNFLGLDFAGGSGGKFTVINGIRDISRG
SIVHPDNRWYKAWTMFILIWALYSSFFTPLEFGFFRGLPENLFILDIAGQIAFLVDIVLTFFVAYRDSRTYRMIY
KRSSIALRYLKSTFIIDLLACMPWDIIYKAAGEKEEVRYLLLIRLYRVHRVILFFHKMEKDIRINYLFTRIVKLIFVEL
YCTHTAACIFYYLATTLPASQEGYTWIGSLKLGDYSYSKFREIDLWTRYTTSMYFAVVTMATVGYGDIHAVN
MREMIFAMVYISFDMILGAYLIGNMTALIVKGSKTERFRDKMADIMRYMNRNKLGRNIRGQITGHLRLQYE
SSYTEAAVLQDIPVSIRAKIAQTLYLPYIEKVPLFRGCSSEFINQIVIRLHEEFFLPGEVIMEQGSVVDQLYFVCH
GVLEEIGITKDGSEEIVAVLQPDHSFGEISILCNIPQPYTVRVAELCRILRLDKQSFMNILEIFFHDGRRILNNLLE
GKESNVRIKQLESDITFHISKQEAELALKLNSAAFYGDLYQLKSLIRAGGDPNKTDYDGRSPLHLAASRGYEDIT
LYLIQESVDVNIKDKLGSTPLLEAIKNGNDRVAALLVKEGATLNIENAGTFLCTVVAKGDSDFLKRLLSNGIDP
NSKDYDHRTPLHVAASEGFYVLAIQLVEASANVLAKDRWGNTPLDEALGCGNKMLIKLLEDAKNSQISSFPS
GSKEPKDKVYKKKCTVYFSHPGDSKEKRRRGIVLWVPRSIEELIRTAKEQLNVPEASCVLSEDEAKIIDVDLISD
GQKLYLAVET
>A. thaliana SKOR mRNA
SEQ ID NO: 12
ATGGGAGGTAGTAGCGGCGGCGGAGTTTCTTATCGTAGCGGCGGCGAATCAGACGTGGAATTAGAGG
ATTACGAGGTTGATGATTTCAGAGATGGGATTGTAGAATCGCGAGGAAACAGATTCAATCCTCTCACCA
ATTTCTTAGGGTTAGACTTCGCCGGCGGTAGCGGTGGAAAGTTCACCGTCATTAATGGAATCAGAGAT
ATCTCCAGAGGCTCCATTGTTCATCCCGATAACCGGTGGTACAAGGCGTGGACGATGTTTATATTGATA
TGGGCACTTTATTCTTCCTTCTTCACTCCATTGGAATTCGGATTCTTCAGGGGATTACCAGAGAATCTGTT
CATCCTCGATATCGCTGGCCAAATCGCTTTCTTAGTAGATATTGTCTTGACATTCTTCGTTGCTTATCGTG
ATAGCCGAACTTATAGAATGATCTATAAACGCAGCTCAATTGCTTTACGGTACTTAAAATCAACTTTTAT
AATTGACTTACTTGCTTGCATGCCATGGGATATCATCTACAAGGCTGCAGGCGAAAAAGAAGAAGTGA
GATACCTATTGTTGATAAGGCTATATCGAGTTCATAGAGTAATCCTGTTTTTCCACAAAATGGAGAAAG
ATATAAGAATCAATTACCTTTTTACAAGAATCGTCAAGCTTATATTCGTCGAGCTTTATTGCACTCACACC
GCAGCTTGTATCTTCTATTACTTGGCCACGACGCTTCCTGCTTCTCAAGAAGGGTACACTTGGATTGGAA
GCTTGAAGTTAGGAGATTACAGTTACTCGAAGTTTAGAGAGATCGATCTCTGGACTCGATACACTACTT
CTATGTACTTTGCAGTTGTTACTATGGCAACTGTTGGTTATGGAGATATACACGCGGTTAATATGCGGG
AAATGATATTCGCGATGGTCTACATTTCATTCGATATGATTCTAGGAGCTTACTTGATTGGTAACATGAC
AGCTTTGATTGTAAAAGGTTCGAAAACAGAGAGATTCCGGGATAAGATGGCTGATATCATGCGGTATA
TGAACCGAAACAAACTCGGTAGGAACATCCGTGGTCAGATTACCGGACATTTGCGGTTGCAGTACGAA
AGCAGCTACACTGAAGCAGCTGTTCTTCAAGACATTCCTGTCTCTATCCGTGCTAAGATTGCGCAAACTT
TATACTTGCCGTATATTGAGAAAGTTCCTCTCTTTCGCGGATGCTCATCTGAATTCATTAACCAGATTGTT
ATAAGACTTCATGAAGAGTTCTTTCTTCCTGGAGAAGTTATAATGGAGCAAGGAAGCGTCGTTGATCAG
CTCTACTTTGTTTGTCACGGTGTATTGGAGGAGATAGGTATAACAAAGGATGGATCTGAAGAAATAGT
GGCAGTTCTACAACCAGATCATTCTTTTGGAGAGATTTCAATCCTCTGCAATATTCCTCAGCCTTACACG
GTTAGAGTTGCAGAGCTATGTCGGATTCTAAGGCTTGACAAGCAATCTTTCATGAACATACTCGAGATA
TTTTTCCATGATGGACGGAGGATTCTCAACAATCTGCTTGAAGGGAAAGAATCTAATGTCCGGATTAAG
CAACTAGAATCCGATATAACCTTTCATATCAGTAAACAAGAGGCAGAGCTAGCTCTGAAGTTGAATAGT
GCGGCTTTCTACGGTGATCTTTATCAGCTTAAGAGTTTGATTCGAGCTGGAGGTGACCCGAATAAGACA
GATTATGACGGAAGATCGCCTTTGCATCTTGCAGCCTCTAGAGGATATGAAGATATTACATTATATCTTA
TACAAGAATCAGTTGATGTAAATATCAAAGATAAATTGGGGAGCACACCTTTACTAGAAGCAATCAAG
AATGGGAATGATCGTGTTGCGGCGTTGCTTGTTAAAGAAGGAGCTACGTTGAACATAGAGAACGCAG
GGACTTTCCTTTGCACCGTGGTTGCGAAAGGAGACAGCGATTTCTTGAAACGGCTTCTTAGTAACGGAA
TTGATCCTAATTCCAAAGATTATGATCACAGAACACCTCTTCATGTAGCTGCCTCTGAAGGATTTTATGT
CTTGGCGATCCAATTGGTAGAAGCAAGTGCTAATGTTCTTGCAAAAGACAGATGGGGAAACACACCTC
TTGATGAAGCTTTGGGTTGTGGGAACAAGATGTTGATAAAGTTACTCGAAGACGCTAAGAATTCTCAA
ATCTCTTCGTTTCCGAGTGGCTCCAAAGAGCCTAAAGATAAAGTCTATAAGAAGAAATGTACAGTGTAT
TTTTCACATCCTGGTGATTCGAAAGAGAAAAGAAGGCGCGGGATTGTACTGTGGGTACCTCGAAGCAT
CGAGGAGCTTATAAGAACAGCAAAGGAGCAGCTGAATGTTCCGGAGGCTTCTTGTGTATTATCTGAAG
ATGAAGCCAAAATTATTGATGTAGATTTGATAAGTGATGGACAAAAACTGTATTTGGCTGTTGAAACAT
AA
>Brassica napus SKOR X1_XP_013750920.1
SEQ ID NO: 13
MGGSSGGGVSYRSEVDSDVELEDYEVDDDFGEGIVESRGNRFNPLTNFLGLDFTGGNGGKFTVINGIRDISR
GSVVHPDSGCYKAWTMFIVIWALYSSFFTPLEFGFFRGLPGNLFILDILGQIAFLVDIVLTFFVAYRDSRTYRM
VYRRSSIALRYLKSTFIIDFLSCMPWDIIYKVAGRKEEVRYLLLIRLYRVRRVILFFHKMEKDIRINYLFTRIVKLIFV
ELYCTHTAACIFYYLATTLPASQEGYTWIGSLKLGDYSYAKFREIDLWTRYTTSMYFAVVTMATVGYGDIHAV
NMREMIFAMAYISFDMILGAYLIGNMTALIVKGSKTERFRDKMADIMRYMNRNKLGRNIRGQITGHLRLQ
YESSYTEAAVLQDIPVSIRAKIAQTLYLSYIEKVPLFRGCSSEFINQIVIRLHEEFFLPGEVIMEQGSVVDQLYFVC
HGVLEEIGTAKDGSEEIVSLLQPDNSFGEISILCNIPQPYTVRVSELCRVLRLDKQSFMNILEIYFHDGRRILNNL
LEGKESNGRIKQLESDITFHISKQEAELALKLNSAAFYGDLYQLKSLIRAGADPNKTDYDGRSPLHLAASRGYE
DITLYLIQESVDVNIKDKLGNTPLLEAIKNGNDRVAALLVKEGATLSIENAGTFLCTVVAKGDSDFLKRLLNNGI
DPNSKDYDQRTPLHVASSEGLYLLARQLVEAGANVLKKDRWGNTPLDEALVCGNKMLIKLLEDAKTSQMS
TFLNSSKEIKDKVYKKKCTVYSSHPNDSKEKRRRGIVLWVPKSIEELVRSAAEQLNFPEASCVLSEDEGKIIDVE
LISDGQKLYLTIET
>Brassica napus SKOR X1 mRNA
SEQ ID NO: 14
ATGGGAGGTAGCAGCGGCGGCGGAGTTTCGTACCGGAGCGAAGTCGACTCGGACGTGGAGTTGGAG
GATTACGAGGTGGATGATGATTTCGGAGAAGGCATCGTAGAATCGCGAGGAAACAGATTCAATCCCCT
CACCAATTTCTTAGGATTAGATTTCACCGGAGGTAACGGCGGAAAGTTCACCGTCATTAACGGAATCAG
AGATATCTCCAGAGGCTCCGTTGTTCATCCTGATAGCGGATGCTACAAGGCGTGGACCATGTTCATAGT
GATATGGGCACTGTACTCTTCCTTCTTCACTCCATTGGAGTTCGGATTCTTCAGGGGATTACCAGGGAAT
CTGTTCATCCTCGACATCCTTGGCCAAATCGCTTTCTTAGTCGATATTGTGTTAACATTCTTCGTGGCGTA
TAGAGACAGCAGAACTTACAGAATGGTCTATAGACGCAGCTCCATCGCTTTACGCTACTTAAAATCAAC
TTTTATTATTGATTTTCTATCATGTATGCCATGGGATATCATCTACAAGGTAGCAGGTCGAAAAGAAGAA
GTGAGATACCTATTGTTGATAAGGCTATATCGAGTTCGAAGAGTGATCCTGTTTTTCCACAAAATGGAG
AAAGACATAAGAATCAATTATCTTTTCACAAGAATCGTCAAGCTTATATTCGTCGAGCTCTATTGCACTC
ACACCGCTGCTTGCATCTTCTATTACTTGGCCACCACGCTTCCTGCTTCTCAAGAAGGGTACACTTGGAT
TGGAAGCTTGAAACTGGGGGATTACAGTTACGCTAAGTTTAGAGAGATCGATCTCTGGACCCGTTACA
CCACTTCTATGTACTTCGCTGTTGTCACTATGGCAACTGTTGGTTATGGAGATATACATGCGGTTAACAT
GCGGGAAATGATATTCGCGATGGCCTACATATCATTCGACATGATTCTAGGTGCTTACTTGATCGGTAA
CATGACAGCTTTGATAGTAAAAGGCTCAAAAACAGAGAGATTCAGGGACAAGATGGCGGATATTATGA
GGTATATGAACCGAAACAAACTCGGTAGAAACATCCGTGGTCAGATCACTGGTCATTTGCGGTTGCAG
TACGAAAGTAGTTACACTGAAGCAGCTGTTCTTCAAGACATACCTGTCTCTATCCGCGCTAAGATTGCTC
AAACGTTATATTTGTCGTATATTGAGAAAGTCCCTCTCTTCCGTGGATGCTCATCTGAATTCATTAACCA
GATTGTTATAAGACTTCATGAAGAGTTTTTTCTCCCTGGAGAAGTAATAATGGAGCAAGGAAGTGTTGT
TGATCAACTCTACTTCGTCTGTCATGGTGTATTGGAGGAGATAGGTACAGCTAAAGATGGATCTGAAGA
GATAGTATCACTACTTCAACCAGATAACTCCTTTGGAGAGATTTCAATCCTCTGCAACATTCCTCAGCCTT
ACACAGTTCGAGTTTCCGAGCTGTGTAGGGTTCTAAGGCTAGACAAACAATCTTTTATGAACATACTCG
AGATATATTTCCACGATGGACGGAGGATACTCAACAATCTGCTTGAAGGGAAAGAATCTAACGGCAGG
ATTAAGCAGCTGGAATCTGATATTACCTTTCATATCAGTAAACAAGAGGCAGAGCTAGCTTTGAAGTTG
AATAGTGCTGCTTTCTATGGCGATCTTTATCAGCTTAAGAGCTTGATTCGAGCTGGAGCTGACCCAAAT
AAAACAGATTATGATGGAAGATCGCCTTTGCATCTTGCAGCGTCTAGAGGATATGAAGACATCACACTG
TATCTTATTCAAGAATCGGTAGACGTAAATATCAAAGATAAACTGGGGAACACGCCGTTACTAGAAGCA
ATCAAGAACGGGAACGATCGTGTTGCGGCGTTGCTTGTGAAAGAAGGTGCAACGTTGAGCATAGAGA
ACGCAGGGACTTTTCTTTGCACTGTGGTTGCCAAAGGAGACAGCGATTTCTTAAAACGTCTTCTCAACA
ATGGCATTGATCCAAACTCTAAAGATTATGATCAGAGGACACCTCTTCACGTTGCTTCCTCTGAAGGGTT
GTATCTCTTGGCAAGACAGTTGGTAGAAGCAGGTGCTAATGTTCTTAAAAAGGACAGGTGGGGAAATA
CTCCTCTAGATGAAGCCCTAGTTTGTGGAAACAAGATGTTGATAAAACTACTTGAAGACGCTAAGACTT
CTCAGATGTCTACGTTTCTGAATAGCTCTAAAGAGATCAAAGATAAAGTCTATAAGAAGAAATGCACAG
TGTATTCTTCACATCCGAATGATTCCAAAGAGAAAAGAAGACGAGGGATTGTATTGTGGGTGCCTAAA
AGCATCGAGGAGCTTGTAAGAAGTGCAGCAGAACAGCTGAATTTTCCGGAAGCTTCTTGTGTTTTGTCT
GAAGATGAAGGTAAAATTATTGATGTAGAGTTGATAAGTGATGGACAAAAACTGTATCTGACTATTGA
AACATAA
>Raphanus sativus (Radish) SKOR XP_018491793.1
SEQ ID NO: 15
MGGSSGNGVSYRGEGESDVELEDYEVDDDDFRDGIVETRGNRFNPLTNFLGLDLAGGNGGKFTVINGIRDI
SRGSVIHPDNRCYKAWTTFILIWALYSSFFTPLEFGFFRGLPENLFILDIAGQIAFLVDIVLTFFVAYRDSRTYRM
VYRRSSIALRYLKSSFVIDLLACMPWDIIYKAAGEKEEVRYLLLIRLYRVRRVILFFHKMEKDIRINYLFTRIVKLIF
VELYCTHTAACIFYYLATTLPASQEGYTWIGSLKLGDYSYAKFREIDLWTRYTTSMYFAIVTMATVGYGDIHA
VNMREMIFAMIYISFDMILGAYLIGNMTALIVKGSKTERFRDKMADIMRYMNRNKLSRNIRGQITGHLRLQ
YQSSYTEAAVLQDIPVSIRAKIAQTLYLPYIEKVPLFRGCSSEFINQIVIRLHEEFFLPGEVIMEQGSVVDQLYFV
CHGVLEEIGTAKDGSEEIVSLLQPDNSFGEISILCNIPQPYTVRVSELCRILRLDKQSFMNILEIYFHDGRRILNN
LLEGKESNVRIKELESDITFHISKQEGELALKLNSAAFYGDLYQLKSLIRAGADPNKTDYDGRSPLHLAASRGYE
DITLYLIQESVDVNIKDKLGNTPLLEAIKNGNDRVAALLVKEGATLSIENAGTFLCTVVAKGDSDFLKRLLNNGI
DPNSKDYDHRTPLHVAASEGLYLLAMQLVEAGANVLKKDRWGNTPLDEALGCGNKMLIKLLEDAKSSQISS
FPSSSKELKDKVYKKKCTVYSSHPNDAKETRRRGIVLWVPRNIEELVRTAAEQLNVPEASCVLSEDEGKIIDVD
LISDGQKLYLTVET*
>Raphanus sativus (Radish) SKOR mRNA
SEQ ID NO: 16
ATGGGAGGCAGTAGCGGCAACGGAGTATCGTACCGGGGCGAGGGAGAATCTGACGTGGAGTTGGAG
GATTACGAGGTGGATGATGATGACTTCAGAGATGGGATTGTAGAAACGCGAGGAAACAGATTCAACC
CCCTCACGAACTTCCTAGGGTTAGATTTGGCCGGAGGTAACGGAGGAAAGTTCACCGTCATTAACGGA
ATCAGAGATATCTCCAGAGGCTCCGTTATTCATCCCGATAACCGTTGCTACAAGGCGTGGACCACGTTC
ATACTGATATGGGCACTTTACTCTTCCTTCTTTACTCCACTAGAATTTGGATTCTTCAGGGGACTACCAG
AGAATCTATTCATCCTAGACATTGCAGGCCAAATCGCTTTCTTAGTCGATATCGTGCTAACGTTCTTCGT
GGCTTATAGAGATAGCAGAACTTACAGAATGGTCTATAGACGCAGCTCCATCGCTCTACGGTACTTAAA
ATCATCTTTTGTTATTGATTTACTTGCTTGCATGCCATGGGATATCATTTACAAGGCTGCAGGTGAAAAA
GAAGAAGTGAGATACCTATTGTTGATAAGACTATATCGAGTTCGTAGAGTAATCCTGTTCTTCCACAAA
ATGGAGAAAGACATAAGAATCAATTACCTTTTCACAAGAATCGTCAAGCTTATATTTGTTGAGCTCTATT
GCACTCACACCGCGGCTTGTATCTTCTATTACCTGGCCACCACGCTACCTGCTTCTCAAGAAGGGTATAC
TTGGATTGGAAGCTTGAAACTGGGAGATTACAGTTACGCTAAGTTTAGAGAGATCGATCTCTGGACTC
GGTACACAACTTCTATGTACTTTGCAATTGTCACTATGGCAACTGTTGGTTATGGAGATATACATGCGGT
TAATATGCGGGAAATGATATTTGCGATGATCTACATATCATTCGACATGATTCTAGGTGCTTACTTGATC
GGTAACATGACAGCTTTGATAGTAAAAGGTTCAAAAACAGAGAGATTCAGGGACAAGATGGCGGATA
TTATGAGGTATATGAACCGAAACAAACTCAGTAGAAACATCCGTGGTCAGATCACTGGACATTTACGGT
TGCAGTACCAAAGTAGTTACACCGAGGCAGCTGTTCTTCAAGACATACCTGTCTCTATCCGCGCCAAGA
TTGCACAAACTTTATACTTGCCATATATTGAGAAGGTTCCTCTCTTCCGTGGCTGCTCATCTGAGTTCATT
AACCAGATTGTTATAAGACTTCATGAAGAGTTCTTTCTCCCTGGAGAAGTTATCATGGAGCAAGGAAGC
GTTGTGGATCAGCTCTACTTTGTTTGTCATGGTGTTTTGGAGGAGATAGGTACAGCTAAGGATGGATCA
GAAGAGATAGTGTCACTTCTTCAACCAGATAACTCTTTTGGAGAGATATCAATCCTCTGCAACATTCCTC
AGCCTTACACAGTTCGAGTTTCTGAGCTGTGTCGGATTCTAAGACTAGATAAACAATCTTTCATGAACAT
CCTCGAGATATATTTCCATGACGGAAGGAGGATCCTCAACAATCTGCTTGAAGGGAAAGAATCTAATGT
CCGGATTAAGGAGCTGGAATCTGATATTACATTTCATATCAGTAAACAAGAGGGAGAGCTAGCTTTGA
AGTTGAACAGTGCTGCTTTCTACGGTGATCTTTACCAGCTTAAGAGCTTGATTCGAGCTGGAGCTGACC
CGAATAAGACAGATTATGACGGAAGATCACCTTTGCATCTTGCAGCCTCTAGAGGATATGAAGACATA
ACATTGTACCTAATCCAAGAATCAGTAGATGTAAATATCAAAGATAAACTAGGGAACACGCCGTTACTA
GAAGCAATCAAGAACGGGAATGATCGTGTCGCGGCTTTGCTTGTGAAAGAAGGTGCAACGTTGAGCAT
AGAGAACGCAGGGACTTTCCTTTGCACGGTGGTTGCTAAAGGAGACAGCGATTTCTTGAAACGTCTTCT
CAACAACGGCATTGATCCTAACTCTAAAGACTATGATCACAGGACACCTCTCCACGTTGCTGCTTCTGAA
GGATTGTATCTATTAGCAATGCAACTAGTAGAAGCAGGTGCTAATGTTCTTAAAAAAGACAGGTGGGG
GAATACTCCTTTAGACGAAGCCCTAGGTTGTGGTAATAAGATGTTGATAAAACTACTTGAAGACGCTAA
GAGTTCACAGATCTCTTCGTTTCCAAGTAGCTCCAAAGAGCTCAAAGATAAAGTATACAAGAAGAAATG
TACAGTGTATTCTTCACATCCGAATGACGCAAAAGAAACAAGAAGACGTGGGATTGTGTTGTGGGTGC
CTAGAAACATCGAGGAGCTTGTGAGAACTGCAGCAGAACAGTTGAATGTTCCTGAAGCTTCTTGTGTGT
TGTCTGAAGATGAAGGTAAAATCATTGATGTAGATTTGATTAGTGATGGACAAAAGCTGTATCTGACCG
TTGAAACATAA
>Glycine max SKOR XP_003544361.1
SEQ ID NO: 17
MTGKKNVKAATTTTPEEEDQRKQTTTSSSPSSSSRSSSSSSSSDEREYEVQDLRDRLKSSRGSRFDLIENQLGL
NSTWSKFSRQALLHGIRGFSVDFVIHPDNRWYRAWTKFILLWAVYSSFFTPMEFGFFRGLPENLFILDIIGQI
AFLVDIVLQFFVAYRDSQTYRTVYKRTPIALRYLKSNFIFDLLGCMPWDIIYKACGRKEEVRYLLWIRLYRVRKV
TDFFHKLEKDIRVNYIITRIVKLIVVELYCTHTAACIFYYLATTLPESQEGYTWIGSLKLGDFSYSHFREIDLWKRY
TTSLYFAIVTMATVGYGDIHAVNMREMVFIMVYVSFDMILGAYLIGNMTALIVKGSKTEKFRDKMTDLMKY
MNRNRLGRDIREQIKGHVRLOYESSYTEASVIQDIPISIRAKISQTLYLPYIEKVSLFKGCSSEFIRQIVIRLHEEFF
LPGEVIMEQGNVVDQLYFVCHGVLEEVGTAEDGTEETVSLLQPNSSFGEISILCNIPQPYTVRVCELSRLLRLD
KQSFTNILDIYFYDGRKVLNNLLEGKESFRDKQLESDITFHIGKQEAELALKVNNAAFNGDLYQLKGLIRAGAD
PNKTDYDGRSPLHLAASRGYEDITLFLIQERVDVNIKDNFGNTPLLEAVKNGHDRVASLLVREGASMKIENA
GSFLCTAVARGDSDYLKRLLSNGMDPNLKDYDYRSPLHIAAAEGLYFMAKLLLEGGASVFTKDRWGNTPLD
EARMCGNKNLIKLLEDAKSAQLSEFPSQEYTDKMHPKKCTVFPYHPWDPKDNRRHGIVLWIPHSIQELIKSA
AEQIEFSGDACILSEDAGKVTDVDMIKDGQKLYLVHETH
>Glycine max SKOR XP_003544361.1 mRNA
SEQ ID NO: 18
ATGACGGGGAAGAAGAATGTGAAGGCGGCGACAACCACGACACCGGAAGAGGAAGACCAGAGGAA
GCAAACGACGACGTCGTCTTCCCCCTCGTCGTCTTCGCGCTCCTCCTCCTCGTCGTCGTCTTCGGATGAG
AGAGAGTACGAGGTGCAGGACCTGCGTGACCGGTTGAAATCGTCGCGAGGGAGCAGGTTCGATCTCA
TAGAGAACCAGTTGGGACTCAACTCGACTTGGAGCAAGTTCAGTCGCCAAGCGCTTCTCCATGGAATCC
GAGGCTTCTCCGTGGACTTCGTTATTCATCCTGATAACAGGTGGTATCGGGCATGGACAAAATTCATAT
TGCTATGGGCAGTGTATTCATCCTTCTTCACCCCAATGGAGTTTGGGTTTTTCCGTGGCCTGCCTGAGAA
TCTCTTTATTCTGGACATCATTGGACAAATAGCTTTTCTTGTAGATATTGTCTTGCAATTTTTTGTTGCTTA
TAGAGATAGTCAGACCTATCGTACGGTCTACAAGAGAACCCCTATTGCTCTGCGGTATCTGAAATCTAA
CTTCATATTTGATCTTCTTGGATGTATGCCCTGGGACATCATCTACAAGGCTTGTGGAAGAAAAGAGGA
AGTGAGATACCTTTTGTGGATCAGACTATATAGGGTTCGGAAAGTCACAGATTTTTTCCACAAGTTGGA
GAAGGACATACGTGTCAATTACATTATTACCCGGATTGTGAAACTGATAGTAGTTGAACTCTATTGCAC
CCATACAGCAGCCTGCATTTTCTACTATTTGGCTACCACACTACCTGAATCCCAAGAAGGGTACACATGG
ATAGGAAGTTTAAAACTAGGTGACTTTAGTTATTCACATTTCAGAGAAATTGATCTTTGGAAGCGCTAT
ACAACATCTCTGTACTTCGCTATTGTTACAATGGCTACAGTTGGCTATGGAGATATACATGCAGTAAATA
TGAGGGAGATGGTATTCATAATGGTGTATGTTTCATTTGATATGATTCTTGGTGCCTATTTGATTGGTAA
CATGACAGCTTTGATAGTCAAGGGATCAAAGACTGAAAAGTTTAGGGACAAGATGACAGACCTTATGA
AATATATGAACAGAAATAGGCTTGGAAGGGATATCCGTGAACAAATTAAGGGCCATGTGCGGTTACAG
TATGAGAGTAGCTACACTGAGGCTTCTGTCATTCAGGATATCCCTATATCTATTCGAGCTAAGATATCCC
AAACATTATATTTGCCGTACATTGAAAAGGTTTCTCTTTTTAAGGGATGCTCTTCTGAATTCATCCGTCA
GATTGTTATTAGGCTCCATGAGGAGTTCTTTCTTCCAGGGGAAGTTATAATGGAACAAGGAAATGTTGT
GGATCAACTATATTTTGTCTGCCATGGAGTACTGGAGGAAGTAGGCACTGCTGAAGATGGGACTGAAG
AAACTGTTTCACTTCTGCAGCCTAACAGTTCATTCGGAGAAATATCTATTCTTTGCAACATTCCTCAACCA
TATACTGTCCGTGTTTGTGAACTGAGTAGACTCCTGCGACTTGATAAACAATCATTTACAAATATCCTAG
ACATATACTITTATGATGGAAGGAAAGTATTAAACAACCTTCTAGAGGGAAAAGAATCTTTTCGAGATA
AGCAGTTGGAGTCAGACATCACATTTCATATTGGAAAGCAAGAAGCTGAGCTTGCTTTGAAGGTCAAT
AATGCAGCTTTCAATGGGGATCTGTATCAGCTAAAAGGTTTAATTCGCGCTGGAGCTGATCCCAACAAG
ACAGATTATGATGGAAGGTCACCTTTGCATCTTGCAGCATCTAGAGGATATGAAGATATCACACTTTTC
CTTATTCAAGAACGTGTAGATGTCAATATTAAAGATAACTTCGGGAACACACCCTTACTTGAAGCAGTT
AAGAATGGACATGATCGGGTTGCTTCTTTACTTGTTAGAGAAGGGGCCTCTATGAAGATTGAAAATGCT
GGTAGTTTTTTGTGTACTGCAGTTGCAAGGGGAGATTCAGATTATCTTAAAAGACTITTATCCAATGGC
ATGGATCCTAACTTAAAAGATTATGATTACCGAAGTCCTCTTCACATTGCTGCAGCTGAAGGTTTATATT
TCATGGCAAAGTTGCTATTAGAAGGAGGAGCTAGTGTTTTTACCAAAGACAGATGGGGAAATACGCCG
CTTGATGAAGCTCGGATGTGTGGAAATAAGAACCTGATCAAGCTTCTGGAAGATGCAAAATCTGCTCA
ACTGTCAGAATTCCCATCCCAAGAATATACAGACAAAATGCATCCAAAGAAGTGCACAGTGTTCCCTTA
CCACCCGTGGGATCCGAAAGATAATAGAAGACATGGAATTGTATTATGGATTCCACACTCCATACAAGA
GCTAATCAAATCAGCGGCAGAACAAATAGAATTTTCTGGCGATGCTTGTATTTTATCAGAAGATGCTGG
TAAAGTTACTGATGTGGACATGATCAAGGATGGTCAGAAGCTGTATTTAGTCCATGAAACACATTAG
>Solanum lycopersicum SKOR XP_004240037.1
SEQ ID NO: 19
MSMKRELREERNGRDSPKEYKMDDLRDSMKSLRSTSRLAMMENELIADSTPWRFSSENVLNGLRGLSQGF
VIYPDDRWYKLWDKFILIWAIYSTFFTPMEFGFFKGLPRKLFLLDICGQIAFLVDIVIQFFVAYRDSQTYKMVY
RRTPIALRYLKSHFILDVLSCMPWDNIYKASGRKEGVRYLLWIRLSRVRRVTDFFQKMEKDIRINYLFTRIVKLI
TVELYCTHTAACIFYFLATTLPEEKEGYTWIGSLTLGDYSYSHFREIDLWRRYITSLYFAIVTMATVGYGDIHAV
NLREMIFVMVYVSFDMILGAYLIGNMTALIVKGSKTVRYRDKMTDLMNYMNRNRLGRDIRSQIKDHLRLQ
YESAYTDGAVLQDLPISIRAKISQTLYLSCIENIPLFRECSAEFISQIVTRVHEEFFLPGEVIMEQGHVVDQLYFV
CDGVLEEVGIGEDGSQETVALLEPNSSFGEISILCNIPQPYTVRVSELCRLIRIDKQSFSNILEIYFHDGRRILTNL
LEGKDLRVKQLESDITFHIGKQEAELALKVNSAAYHGDLHQLKSLIRAGADPNKKDYDGRSPLHLSASRGYED
ISIFLIKEGVDFNASDNFGNTPLFEAIKNGHDRVASLLVKEGAFLKIENAGSFLCTLVTKGDSDLLRRLLSNGID
ANSKDYDHRTPLHVAASQGLLAMARLLLGAGASVFSKDRWGNTPFDEARLSGNNQLIKLLEEAKSAQTSEI
HSVSHEISEKIHLRKCTVYPIHPWEPKDLRKHGVVLWVPTSMEELVTAASEQLNFSSGSCILSEDAGKILDIDM
ISDGQKLYLISETT
>Solanum lycopersicum SKOR mRNA
SEQ ID NO: 20
ATGTCGATGAAGAGAGAATTACGAGAAGAGAGAAATGGAAGAGATTCACCAAAAGAGTATAAAATGG
ATGATTTGAGAGATAGTATGAAATCGTTGCGAAGTACCAGTCGATTGGCGATGATGGAAAACGAGCTA
ATTGCCGATTCAACTCCTTGGAGATTTAGCAGTGAAAACGTCCTCAATGGCCTCAGAGGCCTTTCTCAA
GGTTTCGTCATTTATCCCGATGACAGGTGGTACAAGCTATGGGATAAGTTTATCCTGATTTGGGCTATA
TATTCAACTTTTTTTACACCAATGGAGTTTGGATTCTTCAAAGGATTGCCCAGGAAACTATTTCTTTTAGA
CATTTGTGGTCAAATAGCTTTTCTTGTCGACATAGTCATACAATTTTTTGTAGCCTACAGAGACAGTCAG
ACATACAAGATGGTGTATAGGCGAACCCCTATTGCTCTCCGGTACTTGAAATCTCATTTTATCTTGGATG
TGCTCAGTTGCATGCCGTGGGATAACATTTATAAGGCTTCTGGCAGAAAAGAAGGAGTGAGATACCTT
TTATGGATTAGATTAAGCAGGGTGCGGAGGGTTACTGACTTTTTCCAGAAGATGGAGAAAGATATTCG
GATCAATTATCTCTTCACTAGGATTGTAAAGCTTATCACTGTTGAACTTTATTGCACGCATACAGCAGCT
TGCATCTTTTACTTTCTGGCAACTACTCTGCCTGAAGAGAAAGAAGGCTACACATGGATTGGTAGTTTG
ACCCTGGGAGATTACAGTTATTCACACTTTAGAGAGATTGATCTCTGGAGGCGGTACATCACTTCCCTG
TACTTTGCAATTGTCACTATGGCAACTGTGGGTTATGGTGACATACATGCAGTCAATCTGAGGGAAATG
ATATTTGTAATGGTTTATGTCTCTTTTGATATGATTCTTGGTGCTTACTTGATCGGTAACATGACAGCATT
GATTGTCAAGGGATCAAAAACTGTGCGATACAGGGACAAAATGACAGATCTTATGAACTATATGAACA
GAAATAGACTCGGAAGGGATATTCGTAGTCAAATTAAAGATCACTTGCGATTACAATATGAAAGCGCTT
ACACTGATGGAGCCGTTCTCCAGGACCTTCCCATCTCAATTCGTGCAAAGATATCCCAGACTTTATATCT
GTCTTGCATTGAAAATATTCCTCTTTTCAGGGAGTGCTCCGCAGAATTCATAAGTCAAATTGTAACTCGA
GTGCATGAAGAATTTTTCCTCCCAGGAGAAGTGATCATGGAACAGGGGCATGTAGTGGACCAACTTTA
TTTTGTCTGTGATGGTGTGCTGGAAGAAGTTGGTATAGGAGAAGATGGGTCACAAGAGACAGTAGCAC
TTCTTGAACCTAATAGCTCGTTTGGAGAAATATCCATTCTTTGCAACATTCCACAGCCATATACTGTCCGT
GTTAGTGAACTATGCAGACTCATACGGATTGATAAGCAATCATTTTCAAATATTTTGGAGATCTATTTTC
ATGATGGAAGAAGAATCTTGACGAACTTACTAGAGGGAAAGGATCTTCGTGTGAAGCAACTGGAGTCA
GATATAACATTCCATATTGGGAAACAAGAGGCTGAGCTTGCTTTGAAAGTGAATAGTGCAGCTTATCAT
GGTGATTTGCACCAGCTGAAGAGTCTGATTCGAGCTGGAGCTGATCCCAACAAGAAAGATTATGATGG
AAGATCACCTCTGCATCTTTCAGCATCCAGAGGATATGAAGACATCTCTATTTTCCTTATCAAAGAAGGT
GTCGATTTCAATGCTTCAGACAACTTTGGAAACACACCGCTCTTTGAAGCTATCAAGAATGGACACGAT
CGTGTTGCTTCATTACTTGTTAAGGAAGGTGCCTTCTTGAAGATTGAAAATGCTGGTAGCTTCTTGTGCA
CATTGGTTACAAAAGGGGATTCAGATCTACTACGAAGGCTGTTGTCCAACGGTATTGATGCAAACTCCA
AAGATTATGATCACCGAACACCGCTCCATGTAGCGGCTTCTCAAGGATTACTCGCAATGGCGAGATTGC
TTCTGGGAGCTGGTGCTTCAGTTTTTTCAAAGGACAGATGGGGAAATACTCCATTTGATGAAGCTAGAC
TAAGTGGAAACAATCAGTTGATCAAGCTTCTGGAAGAAGCAAAATCTGCTCAAACATCGGAAATCCAC
AGTGTTTCACACGAGATTTCAGAAAAAATTCACCTGCGGAAGTGTACTGTATACCCTATCCACCCATGG
GAGCCTAAGGACCTCAGGAAACATGGTGTTGTTTTATGGGTACCTACGAGTATGGAAGAGCTCGTTAC
TGCAGCTTCGGAACAACTCAACTTCTCATCAGGCTCTTGTATTTTATCAGAAGATGCAGGTAAAATTCTT
GATATAGATATGATATCTGATGGTCAGAAGTTGTACTTGATCAGTGAAACAACTTGA
>Solanum tuberosum SKOR XP_006360380.1
SEQ ID NO: 21
MSMKRELGEERNGRDSPKEYKMNDLRDSMKSLRSTSRLAMMENELIADSTPSRFSRENVINGLRGLSQDF
VIYPDDRWYKLWDKFILIWAIYSTFFTPMEFAFFKGLPRKLFLLDICGQIAFLVDIVIQFFVAYRDSQTYKMVY
RRTPIALRYLKSHFILDVLSCMPWDNIFKASGRIEGVRYLLWIRLSRVRRVTDFFQKMEKDIRINYLFTRIIKLIT
VELYCTHTAACIFYFLATTLPAEKEGYTWIGSLTLGDYSYSNFREIDLWRRYITSLYFAIVTMATVGYGDIHAVN
LREMIFVMVYVSFDMILGAYLIGNMTALIVKGSKTVRYRDKMTDLMNYMNRNRLGRDIRSQIKDHLRLQYE
SAYTDGAVLHDLPISIRAKISQTLYQSCIENIPLFRECSAEFISQIVTRVHEEFFLPGEVIMEQGHVVDQLYFVCD
GVLVEVGIGEDGSQETVAHLEPNSSFGEISILCNIPQPYTVRVCELCRLIRIDKQSFSNILEIYFHDGRRILANLLE
GKDLRVKQLESDITFHIGKQEADLALKVNSAAYHGDLHQLKSLIRAGADPNKKDYDGRSPLHLSASRGYEDIS
FFLVKEGIDLNASDNFGNTPLFEAIKNGHDRVASLLVKEGAFLKIENAGSFLCTLVTKGDSDLLRRLLSNGIDA
NSKDYDHRTPLHVAASQGLLAMARLLLGAGASVFSMDRWGNTPFDEARLSGNNQLIKLLEDAKSAQTSEF
PSVSHEISEKKHPRKCTVFPVHPWEPKDLRKHGVVLWVPTSMEELVTAASEQLNFPSGSCILSEDAGKILDID
MISDGQKLYLISETT
>Solanum tuberosum SKOR mRNA
SEQ ID NO: 22
ATGTCGATGAAGAGAGAATTAGGAGAAGAGAGGAATGGAAGAGATTCACCAAAAGAGTATAAGATG
AATGATTTGAGAGATAGTATGAAATCGTTGAGAAGTACCAGTAGATTAGCGATGATGGAGAACGAGCT
AATAGCTGATTCAACTCCTTCGAGATTTAGCAGAGAAAACGTCATCAATGGCCTCAGAGGCCTTTCTCA
AGATTTCGTCATTTATCCCGATGACAGGTGGTACAAGCTATGGGATAAGTTTATCCTGATTTGGGCAAT
ATATTCAACTTTTTTTACACCAATGGAGTTTGCATTCTTTAAAGGATTGCCCAGGAAACTATTTCTTTTAG
ACATTTGTGGTCAAATAGCTTTTCTTGTCGACATAGTCATACAATTTTTTGTAGCCTACAGAGATAGTCA
GACATACAAGATGGTGTATAGGCGAACCCCTATTGCTCTTCGGTACTTGAAATCTCATTTTATCTTGGAT
GTTCTCAGTTGCATGCCGTGGGATAACATTTTTAAGGCTTCTGGAAGAATTGAAGGAGTGAGATACCTT
TTATGGATTAGGTTAAGCAGGGTGCGCAGGGTTACTGACTTTTTCCAGAAGATGGAGAAAGATATTCG
AATCAATTATCTCTTCACTAGGATTATAAAGCTTATCACTGTTGAACTTTATTGCACGCATACAGCAGCTT
GCATCTTTTACTTTTTGGCAACTACTCTGCCTGCAGAGAAAGAAGGCTACACATGGATTGGTAGTTTGA
CCCTGGGAGATTACAGTTATTCAAACTTTAGAGAGATTGATCTCTGGAGGCGGTACATCACTTCTCTGT
ACTTTGCAATTGTCACTATGGCAACTGTGGGTTATGGTGACATACATGCAGTCAATCTGAGGGAAATGA
TATTTGTGATGGTTTATGTTTCTTTTGATATGATTCTTGGTGCTTACTTGATCGGTAACATGACAGCACTG
ATTGTCAAGGGATCAAAAACTGTGCGATACAGGGACAAAATGACAGATCTTATGAACTATATGAACAG
AAATAGACTCGGAAGGGATATTCGTAGTCAAATTAAAGATCACTTGCGATTACAATATGAAAGCGCTTA
CACTGATGGAGCCGTTCTCCATGACCTTCCCATCTCAATTCGTGCCAAGATATCCCAGACTTTATATCAG
TCTTGCATTGAAAATATTCCTCTTTTTAGGGAGTGCTCCGCAGAATTCATAAGTCAAATTGTAACTCGAG
TGCATGAAGAATTTTTCCTCCCAGGAGAAGTGATCATGGAACAGGGGCATGTAGTGGACCAACTTTATT
TTGTCTGTGATGGTGTCCTGGTAGAAGTTGGTATAGGAGAAGATGGGTCACAAGAGACAGTAGCACAT
CTTGAGCCTAATAGCTCGTTCGGAGAAATATCCATTCTTTGCAACATTCCACAACCATATACTGTCCGTG
TTTGTGAACTATGCAGACTCATACGGATTGATAAGCAGTCATTTTCAAATATTTTGGAGATCTATTTTCA
CGATGGAAGAAGAATCTTGGCGAACTTACTAGAGGGAAAGGATCTTCGTGTGAAGCAACTGGAGTCA
GATATAACATTCCATATTGGGAAACAAGAGGCTGATCTTGCTTTGAAAGTTAATAGTGCAGCTTATCAT
GGTGACTTACACCAGCTGAAGAGTCTTATTCGAGCTGGAGCTGATCCCAACAAGAAAGATTATGATGG
AAGATCACCTCTGCATCTTTCAGCATCCAGAGGATATGAAGACATCTCTTTTTTCCTTGTCAAAGAAGGT
ATCGATCTCAATGCTTCAGACAACTTTGGAAACACACCGCTCTTTGAAGCTATCAAGAATGGACACGAT
CGTGTTGCTTCATTACTTGTTAAGGAAGGTGCCTTCTTGAAGATTGAAAATGCTGGTAGCTTCTTGTGTA
CGTTGGTTACAAAAGGGGATTCAGATCTACTACGAAGGCTGTTGTCCAATGGTATTGATGCAAACTCCA
AAGATTATGATCACCGAACACCGCTCCATGTAGCGGCTTCTCAAGGATTGCTTGCGATGGCGAGATTGC
TTCTGGGAGCTGGTGCTTCAGTTTTTTCAATGGACAGATGGGGAAATACTCCATTTGATGAAGCTAGAC
TAAGTGGAAACAATCAGTTGATCAAGCTTCTGGAAGACGCAAAATCAGCTCAAACATCAGAATTCCCCA
GTGTTTCGCATGAGATTTCAGAAAAAAAGCACCCACGGAAGTGTACTGTATTCCCTGTCCACCCATGGG
AGCCCAAGGACCTCAGGAAACATGGTGTTGTACTATGGGTACCTACGAGTATGGAAGAGCTCGTTACT
GCAGCTTCAGAACAACTCAACTTCCCATCAGGCTCTTGTATTTTATCAGAAGATGCAGGTAAAATTCTTG
ATATAGATATGATATCTGATGGTCAGAAGTTGTACTTGATCAGTGAAACAACTTGA
PNNP
SEQ ID NO: 23
MGRLRRRQEIIDHEEEESNDDVSSDDGDLSLAKTFDWLKSSKDDDIKTKGDNKYDYIIDPKNRW
YKAWEMFILVWAIYSSLFTPMEFGFFRGLPERLFVLDIVGQIAFLVDIVLQFFVAYRDTQTYRTVY
KPTRIAFRYLKSHFLMDFIGCFPWDLIYKASGKHELVRYLLWIRLFRVRKVVEFFQRLEKDTRIN
YLFTRILKLLFVEVYCTHTAACIFYYLATTLPPENEGYTWIGSLKLGDYSYENFREIDLWKRYTTA
LYFAIVTMATVGYGDIHAVNLREMIFVMIYVSFDMVLGAYLIGNITALIVKGSNTERFRDKMNDLIS
FMNRKKLGRDLRSQITGHVRLQYDSHYTDTVMLQDIPASIRAKIAQLLYLPYIKKVPLFKGOSTE
FINQIVIRLHEEYFLPGEVITEQGNVVDHLYFVCEGLLEALVTKTDGSEESVTLLGPHTSFGDISII
CNISQPFTVRVCELCHLLRLDKQSFSNILEIYFHDGRTILNNIMEEKESNDRIKKLESDIVIHIGKQ
EAELALKVNSAAFQGDFYQLKSLIRSGADPNKTDYDGRSPLHLAACRGYEDITLFLIQEGVDVN
LKDKFGHTPLFEAVKAGQEGVIGLLAKEGASFNLEDSGNFLCTTVAKGDSDFLKRLLSSGMDP
NSEDYDHRTPLHVAASEGLFLMAKMLVEAGASVISKDRWGNSPLDEARLCGNKKLIKLLEDTK
NAQSSIYPTSLRELQEERIERRKCTVFPFHPQEAKEERSRKHGVVVWIPSNLEKLIVTAAKELGL
SDGASFVLLSEDQGRITDIDMISDGHKLYMISDTTDQTHPAFLYKVVMGRPRRSTRVDSRYPTS
PNNP
SEQ ID NO: 24
atgggacgtctccggagacggcaagagataatagatcatgaagaagaggaatcaaacgacgacgtttcatcagatgatggagatc
tcagtttagccaaaacgtttgattggcttaaatcatcaaaagatgatgatattaaaaccaaaggtgataataaatatgattacatcattgat
cccaaaaacaggtggtacaaggcatgggaaatgtttatattggtgtgggcaatatactcctcattgttcactcccatggagtttggtttcttccg
cggtctgcctgagagactctttgtacttgacattgttggtcagatcgcgttcttggtcgatattgttcttcagttctttgttgcctatcgcgata
ctcagacctaccggactgtctacaaaccaacacgtattgctttccggtacttgaagtcgcattttctcatggatttcatcggttgcttcccttg
ggatcttatttataaggcatcagggaaacatgagttggtgaggtacttgttgtggataaggctatttcgggttcgcaaagtggttgagttttt
ccaaaggcttgagaaagacacaagaatcaactatctattcactagaatcttaaagctcttgttcgttgaagtttattgtactcacactgctg
cttgtatcttctattacttggccaccactcttcctccggaaaacgaaggttacacttggatcggtagcttgaagctaggagactatagctac
gagaatttccgagaaatcgatctatggaaacgttatactactgctctatacttcgccattgtcactatggcaactgtcggttatggagacat
tcacgcggtgaatctgagggaaatgatatttgtaatgatatatgtttcatttgatatggttctcggtgcttaccttattggtaacatcactgcc
ttgattgtgaaaggttcaaacacagagaggttcagagataaaatgaatgatctcataagtttcatgaaccgcaaaaaactcgggagag
accttcgtagccagataactggtcatgttagattgcagtacgacagtcactacaccgacactgtcatgcttcaggacatcccagcatca
atccgcgccaagattgcgcaattattgtatctgccttacatcaaaaaagttcctctcttcaaaggctgctccacagagtttattaatcaaat
agttataaggctccatgaagagtattttcttccaggagaagtaataacagagcaaggaaacgtcgtggatcatttgtatttcgtctgtgaa
ggcttactggaggctcttgttacaaaaacagatggatcagaagagagtgtgacgttacttgggcctcacacttcttttggagacatctcc
atcatttgcaacatttctcaacctttcactgttagggtttgtgagctatgccatcttttacgactcgataaacagtctttctcaaacatcctcga
gatttattttcacgacggacgcacaatcttgaacaatattatggaggagaaggaatcaaatgataggataaagaagctagaatctgac
atagtgattcacattgggaaacaagaagcagaacttgcattgaaagtaaacagtgcagctttccaaggagatttttaccagcttaaga
gcttaatccgatctggagccgatcctaacaaaaccgattacgatggaagatcaccgcttcatcttgcggcatgtagaggctatgaaga
cattacattattccttattcaagaaggtgttgatgtcaatctaaaagataagttcggacacacaccattgtttgaggctgtgaaagcagga
caagaaggagtgattggtttgcttgccaaagaaggagcctcctttaatttagaagattcaggaaacttcctttgcacgacagttgctaaa
ggcgactctgattttctcaagagattgctttcaagcggtatggacccaaacagtgaagattatgatcacagaacgccgcttcatgtcgcg
gcttctgaagggttattcttgatggctaaaatgttggttgaagctggagcaagcgttatttctaaagaccggtgggggaattctccgcttga
tgaagctcgattgtgcggaaacaagaaactgattaagttactcgaagatacgaaaaatgctcagtcgtctatctacccgacaagcttg
cgtgaattacaagaggagagaattgagagacggaaatgcacggtgtttccattccacccgcaagaggcgaaagaagagcgtagt
agaaagcacggagttgtggtttggatcccaagcaatctcgagaaactcatagtaaccgctgcgaaagagctagggctatcggatgg
agcctcatttgtactattatccgaagaccaaggtcgtatcacagacattgatatgattagtgatggacacaaattgtatatgatcagtgat
actactgatcaaaca
NP
SEQ ID NO: 25
atgggacgtctccggagacggcaagagataatagatcatgaagaagaggaatcaaacgacgacgtttcatcaagaagaggaaa
actcagtttagccaaaacgtttcggtggcttaaatcatcaaaaaaacggagaattaaaaccaaaggtaaaaataaatataaatacat
cattaaacccaaaaacaggtggtacaaggcatgggaaatgtttatattggtgtgggcaatatactcctcattgttcactcccatggagtttggtt
tcttccgcggtctgcctgagagactctttgtacttgacattgttggtcagatcgcgttcttggtcgatattgttcttcagttctttgttgcctat
cgcgatactcagacctaccggactgtctacaaaccaacacgtattgctttccggtacttgaagtcgcattttctcatggatttcatcggttgc
ttcccttgggatcttatttataaggcatcagggaaacatgagttggtgaggtacttgttgtggataaggctatttcgggttcgcaaagtggtt
gagtttttccaaaggcttgagaaagacacaagaatcaactatctattcactagaatcttaaagctcttgttcgttgaagtttattgtactcac
actgctgcttgtatcttctattacttggccaccactcttcctccggaaaacgaaggttacacttggatcggtagcttgaagctaggagacta
tagctacgagaatttccgagaaatcgatctatggaaacgttatactactgctctatacttcgccattgtcactatggcaactgtcggttatg
gagacattcacgcggtgaatctgagggaaatgatatttgtaatgatatatgtttcatttgatatggttctcggtgcttaccttattggtaacatc
actgccttgattgtgaaaggttcaaacacagagaggttcagagataaaatgaatgatctcataagtttcatgaaccgcaaaaaactcg
ggagagaccttcgtagccagataactggtcatgttagattgcagtacgacagtcactacaccgacactgtcatgcttcaggacatccc
agcatcaatccgcgccaagattgcgcaattattgtatctgccttacatcaaaaaagttcctctcttcaaaggctgctccacagagtttatta
atcaaatagttataaggctccatgaagagtattttcttccaggagaagtaataacagagcaaggaaacgtcgtggatcatttgtatttcgt
ctgtgaaggcttactggaggctcttgttacaaaaacagatggatcagaagagagtgtgacgttacttgggcctcacacttcttttggaga
catctccatcatttgcaacatttctcaacctttcactgttagggtttgtgagctatgccatcttttacgactcgataaacagtctttctcaaac
atcctcgagatttattttcacgacggacgcacaatcttgaacaatattatggaggagaaggaatcaaatgataggataaagaagctaga
atctgacatagtgattcacattgggaaacaagaagcagaacttgcattgaaagtaaacagtgcagctttccaaggagatttttaccagc
ttaagagcttaatccgatctggagccgatcctaacaaaaccgattacgatggaagatcaccgcttcatcttgcggcatgtagaggctat
gaagacattacattattccttattcaagaaggtgttgatgtcaatctaaaagataagttcggacacacaccattgtttgaggctgtgaaag
caggacaagaaggagtgattggtttgcttgccaaagaaggagcctcctttaatttagaagattcaggaaacttcctttgcacgacagttg
ctaaaggcgactctgattttctcaagagattgctttcaagcggtatggacccaaacagtgaagattatgatcacagaacgccgcttcat
gtcgcggcttctgaagggttattcttgatggctaaaatgttggttgaagctggagcaagcgttatttctaaagaccggtgggggaattctc
cgcttgatgaagctcgattgtgcggaaacaagaaactgattaagttactcgaagatacgaaaaatgctcagtcgtctatctacccgac
aagcttgcgtgaattacaagaggagagaattgagagacggaaatgcacggtgtttccattccacccgcaagaggcgaaagaaga
gcgtagtagaaagcacggagttgtggtttggatcccaagcaatctcgagaaactcatagtaaccgctgcgaaagagctagggctatc
ggatggagcctcatttgtactattatccgaagaccaaggtcgtatcacagacattgatatgattagtgatggacacaaattgtatatgatc
agtgatactactgatcaaaca
NP
SEQ ID NO: 26
MGRLRRRQEIIDHEEEESNDDVSSRRGKLSLAKTFRWLKSSKKRRIKTKGKNKYKYIIKPKNRW
YKAWEMFILVWAIYSSLFTPMEFGFFRGLPERLFVLDIVGQIAFLVDIVLQFFVAYRDTQTYRTVY
KPTRIAFRYLKSHFLMDFIGCFPWDLIYKASGKHELVRYLLWIRLFRVRKVVEFFQRLEKDTRIN
YLFTRILKLLFVEVYCTHTAACIFYYLATTLPPENEGYTWIGSLKLGDYSYENFREIDLWKRYTTA
LYFAIVTMATVGYGDIHAVNLREMIFVMIYVSFDMVLGAYLIGNITALIVKGSNTERFRDKMNDLIS
FMNRKKLGRDLRSQITGHVRLQYDSHYTDTVMLQDIPASIRAKIAQLLYLPYIKKVPLFKGOSTE
FINQIVIRLHEEYFLPGEVITEQGNVVDHLYFVCEGLLEALVTKTDGSEESVTLLGPHTSFGDISII
CNISQPFTVRVCELCHLLRLDKQSFSNILEIYFHDGRTILNNIMEEKESNDRIKKLESDIVIHIGKQ
EAELALKVNSAAFQGDFYQLKSLIRSGADPNKTDYDGRSPLHLAACRGYEDITLFLIQEGVDVN
LKDKFGHTPLFEAVKAGQEGVIGLLAKEGASFNLEDSGNFLCTTVAKGDSDFLKRLLSSGMDP
NSEDYDHRTPLHVAASEGLFLMAKMLVEAGASVISKDRWGNSPLDEARLCGNKKLIKLLEDTK
NAQSSIYPTSLRELQEERIERRKCTVFPFHPQEAKEERSRKHGVVVWIPSNLEKLIVTAAKELGL
SDGASFVLLSEDQGRITDIDMISDGHKLYMISDTTDQTHPAFLYKVVMGRPRRSTRVDSRYPTS
KN-gS1-L4
SEQ ID NO: 27
atgtcgatctcttggactcgaaatttcttcgaaagattctgcgtcgaggaatacaatatagacaccataaaacagagtagtttcctctctg
ccgatcttctaccatctcttggagccaggatcaaccaatctactaagctccgcaaacacataatctctcccaaaaacaggtggtacaagg
catgggaaatgtttatattggtgtgggcaatatactcctcattgttcactcccatggagtttggtttcttccgcggtctgcctgagagactcttt
gtacttgacattgttggtcagatcgcgttcttggtcgatattgttcttcagttctttgttgcctatcgcgatactcagacctaccggactgtcta
caaaccaacacgtattgctttccggtacttgaagtcgcattttctcatggatttcatcggttgcttcccttgggatcttatttataaggcatcag
ggaaacatgagttggtgaggtacttgttgtggataaggctatttcgggttcgcaaagtggttgagtttttccaaaggcttgagaaagacac
aagaatcaactatctattcactagaatcttaaagctcttgttcgttgaagtttattgtactcacactgctgcttgtatcttctattacttggcca
ccactcttcctccggaaaacgaaggttacacttggatcggtagcttgaagctaggagactatagctacgagaatttccgagaaatcgatct
atggaaacgttatactactgctctatacttcgccattgtcactatggcaactgtcggttatggagacattcacgcggtgaatctgagggaa
atgatatttgtaatgatatatgtttcatttgatatggttctcggtgcttaccttattggtaacatcactgccttgattgtgaaaggttcaaacac
agagaggttcagagataaaatgaatgatctcataagtttcatgaaccgcaaaaaactcgggagagaccttcgtagccagataactggt
catgttagattgcagtacgacagtcactacaccgacactgtcatgcttcaggacatcccagcatcaatccgcgccaagattgcgcaatt
attgtatctgccttacatcaaaaaagttcctctcttcaaaggctgctccacagagtttattaatcaaatagttataaggctccatgaagagt
attttcttccaggagaagtaataacagagcaaggaaacgtcgtggatcatttgtatttcgtctgtgaaggcttactggaggctcttgttaca
aaaacagatggatcagaagagagtgtgacgttacttgggcctcacacttcttttggagacatctccatcatttgcaacatttctcaaccttt
cactgttagggtttgtgagctatgccatcttttacgactcgataaacagtctttctcaaacatcctcgagatttattttcacgacggacgcac
aatcttgaacaatattatggaggagaaggaatcaaatgataggataaagaagctagaatctgacatagtgattcacattgggaaaca
agaagcagaacttgcattgaaagtaaacagtgcagctttccaaggagatttttaccagcttaagagcttaatccgatctggagccgatc
ctaacaaaaccgattacgatggaagatcaccgcttcatcttgcggcatgtagaggctatgaagacattacattattccttattcaagaag
gtgttgatgtcaatctaaaagataagttcggacacacaccattgtttgaggctgtgaaagcaggacaagaaggagtgattggtttgcttg
ccaaagaaggagcctcctttaatttagaagattcaggaaacttcctttgcacgacagttgctaaaggcgactctgattttctcaagagatt
gctttcaagcggtatggacccaaacagtgaagattatgatcacagaacgccgcttcatgtcgcggcttctgaagggttattcttgatggc
taaaatgttggttgaagctggagcaagcgttatttctaaagaccggtgggggaattctccgcttgatgaagctcgattgtgcggaaaca
agaaactgattaagttactcgaagatacgaaaaatgctcagtcgtctatctacccgacaagcttgcgtgaattacaagaggagagaat
tgagagacggaaatgcacggtgtttccattccacccgcaagaggcgaaagaagagcgtagtagaaagcacggagttgtggtttgg
atcccaagcaatctcgagaaactcatagtaaccgctgcgaaagagctagggctatcggatggagcctcatttgtactattatccgaag
accaaggtcgtatcacagacattgatatgattagtgatggacacaaattgtatatgatcagtgatactactgatcaaaca
KN-gS1-L4
SEQ ID NO: 28
MSISWTRNFFERFCVEEYNIDTIKQSSFLSADLLPSLGARINQSTKLRKHIISPKNRWYKAWEMFI
LVWAIYSSLFTPMEFGFFRGLPERLFVLDIVGQIAFLVDIVLQFFVAYRDTQTYRTVYKPTRIAFR
YLKSHFLMDFIGCFPWDLIYKASGKHELVRYLLWIRLFRVRKVVEFFQRLEKDTRINYLFTRILKL
LFVEVYCTHTAACIFYYLATTLPPENEGYTWIGSLKLGDYSYENFREIDLWKRYTTALYFAIVTM
ATVGYGDIHAVNLREMIFVMIYVSFDMVLGAYLIGNITALIVKGSNTERFRDKMNDLISFMNRKKL
GRDLRSQITGHVRLQYDSHYTDTVMLQDIPASIRAKIAQLLYLPYIKKVPLFKGCSTEFINQIVIRL
HEEYFLPGEVITEQGNVVDHLYFVCEGLLEALVTKTDGSEESVTLLGPHTSFGDISIICNISQPFT
VRVCELCHLLRLDKQSFSNILEIYFHDGRTILNNIMEEKESNDRIKKLESDIVIHIGKQEAELALKV
NSAAFQGDFYQLKSLIRSGADPNKTDYDGRSPLHLAACRGYEDITLFLIQEGVDVNLKDKFGHT
PLFEAVKAGQEGVIGLLAKEGASFNLEDSGNFLCTTVAKGDSDFLKRLLSSGMDPNSEDYDHR
TPLHVAASEGLFLMAKMLVEAGASVISKDRWGNSPLDEARLCGNKKLIKLLEDTKNAQSSIYPT
SLRELQEERIERRKCTVFPFHPQEAKEERSRKHGVVVWIPSNLEKLIVTAAKELGLSDGASFVLL
SEDQGRITDIDMISDGHKLYMISDTTDQTHPAFLYKVVMGRPRRSTRVDSRYPTS
PN
SEQ ID NO: 29
atgggacgtctccggagacggcaagagataatagatcatgaagaagaggaatcaaacgacgacgtttcatcagatgatggagatc
tcagtttagccgagacgtttgattggcttgattcatcagaggacgatgatattgaaaccgatggtgataatgattatgattacatcattgatc
ccaaaaacaggtggtacaaggcatgggaaatgtttatattggtgtgggcaatatactcctcattgttcactcccatggagtttggtttcttccgc
ggtctgcctgagagactctttgtacttgacattgttggtcagatcgcgttcttggtcgatattgttcttcagttctttgttgcctatcgcgatac
tcagacctaccggactgtctacaaaccaacacgtattgctttccggtacttgaagtcgcattttctcatggatttcatcggttgcttcccttggg
atcttatttataaggcatcagggaaacatgagttggtgaggtacttgttgtggataaggctatttcgggttcgcaaagtggttgagtttttcca
aaggcttgagaaagacacaagaatcaactatctattcactagaatcttaaagctcttgttcgttgaagtttattgtactcacactgctgcttg
tatcttctattacttggccaccactcttcctccggaaaacgaaggttacacttggatcggtagcttgaagctaggagactatagctacgag
aatttccgagaaatcgatctatggaaacgttatactactgctctatacttcgccattgtcactatggcaactgtcggttatggagacattcac
gcggtgaatctgagggaaatgatatttgtaatgatatatgtttcatttgatatggttctcggtgcttaccttattggtaacatcactgccttgat
tgtgaaaggttcaaacacagagaggttcagagataaaatgaatgatctcataagtttcatgaaccgcaaaaaactcgggagagacct
tcgtagccagataactggtcatgttagattgcagtacgacagtcactacaccgacactgtcatgcttcaggacatcccagcatcaatcc
gcgccaagattgcgcaattattgtatctgccttacatcaaaaaagttcctctcttcaaaggctgctccacagagtttattaatcaaatagtta
taaggctccatgaagagtattttcttccaggagaagtaataacagagcaaggaaacgtcgtggatcatttgtatttcgtctgtgaaggctt
actggaggctcttgttacaaaaacagatggatcagaagagagtgtgacgttacttgggcctcacacttcttttggagacatctccatcatt
tgcaacatttctcaacctttcactgttagggtttgtgagctatgccatcttttacgactcgataaacagtctttctcaaacatcctcgagattta
ttttcacgacggacgcacaatcttgaacaatattatggaggagaaggaatcaaatgataggataaagaagctagaatctgacatagtg
attcacattgggaaacaagaagcagaacttgcattgaaagtaaacagtgcagctttccaaggagatttttaccagcttaagagcttaat
ccgatctggagccgatcctaacaaaaccgattacgatggaagatcaccgcttcatcttgcggcatgtagaggctatgaagacattaca
ttattccttattcaagaaggtgttgatgtcaatctaaaagataagttcggacacacaccattgtttgaggctgtgaaagcaggacaagaa
ggagtgattggtttgcttgccaaagaaggagcctcctttaatttagaagattcaggaaacttcctttgcacgacagttgctaaaggcgact
ctgattttctcaagagattgctttcaagcggtatggacccaaacagtgaagattatgatcacagaacgccgcttcatgtcgcggcttctga
agggttattcttgatggctaaaatgttggttgaagctggagcaagcgttatttctaaagaccggtgggggaattctccgcttgatgaagct
cgattgtgcggaaacaagaaactgattaagttactcgaagatacgaaaaatgctcagtcgtctatctacccgacaagcttgcgtgaatt
acaagaggagagaattgagagacggaaatgcacggtgtttccattccacccgcaagaggcgaaagaagagcgtagtagaaagc
acggagttgtggtttggatcccaagcaatctcgagaaactcatagtaaccgctgcgaaagagctagggctatcggatggagcctcatt
tgtactattatccgaagaccaaggtcgtatcacagacattgatatgattagtgatggacacaaattgtatatgatcagtgatactactgat
caaaca
PN
SEQ ID NO: 30
MGRLRRRQEIIDHEEEESNDDVSSDDGDLSLAETFDWLDSSEDDDIETDGDNDYDYIIDPKNR
WYKAWEMFILVWAIYSSLFTPMEFGFFRGLPERLFVLDIVGQIAFLVDIVLQFFVAYRDTQTYRT
VYKPTRIAFRYLKSHFLMDFIGCFPWDLIYKASGKHELVRYLLWIRLFRVRKVVEFFQRLEKDTRI
NYLFTRILKLLFVEVYCTHTAACIFYYLATTLPPENEGYTWIGSLKLGDYSYENFREIDLWKRYTT
ALYFAIVTMATVGYGDIHAVNLREMIFVMIYVSFDMVLGAYLIGNITALIVKGSNTERFRDKMNDLI
SFMNRKKLGRDLRSQITGHVRLQYDSHYTDTVMLQDIPASIRAKIAQLLYLPYIKKVPLFKGCST
EFINQIVIRLHEEYFLPGEVITEQGNVVDHLYFVCEGLLEALVTKTDGSEESVTLLGPHTSFGDISI
ICNISQPFTVRVCELCHLLRLDKQSFSNILEIYFHDGRTILNNIMEEKESNDRIKKLESDIVIHIGKQ
EAELALKVNSAAFQGDFYQLKSLIRSGADPNKTDYDGRSPLHLAACRGYEDITLFLIQEGVDVN
LKDKFGHTPLFEAVKAGQEGVIGLLAKEGASFNLEDSGNFLCTTVAKGDSDFLKRLLSSGMDP
NSEDYDHRTPLHVAASEGLFLMAKMLVEAGASVISKDRWGNSPLDEARLCGNKKLIKLLEDTK
NAQSSIYPTSLRELQEERIERRKCTVFPFHPQEAKEERSRKHGVVVWIPSNLEKLIVTAAKELGL
SDGASFVLLSEDQGRITDIDMISDGHKLYMISDTTDQT

1. A genetically altered plant, plant part or plant cell, wherein the plant is characterised by at least one mutation in the N-terminal domain of a stomatal voltage-gated potassium channel.

2. The genetically altered plant, plant part or plant cell of claim 1, wherein the at least one mutation alters the pattern of charged amino acids in the N-terminal domain.

3. The genetically altered plant, plant part or plant cell of claim 2, wherein the mutation is the substitution of at least one positively charged amino acid for a negatively charged amino acid or a neutral amino acid and/or wherein the mutation is the substitution of at least one negatively charged amino acid for a positively charged amino acid or a neutral amino acid.

4. The genetically altered plant, plant part or plant cell of claim 3, wherein the mutation is the substitution of all positively charged amino acids for negatively charged amino acids or neutral amino acids in the N-terminal domain of the stomatal voltage-gated potassium channel.

5. The genetically altered plant, plant part or plant cell of claim 3, wherein the mutation is the substitution of all negatively charged amino acids for positively charged amino acids or neutral amino acids in the N-terminal domain of the stomatal voltage-gated potassium channel.

6. The genetically altered plant, plant part or plant cell of claim 3, wherein the mutation is not the substitution of all positively charged amino acids for negatively charged amino acids or neutral amino acids and the substitution of all negatively charged amino acids for positively charged amino acids or neutral amino acids in the N-terminal domain of the stomatal voltage-gated potassium channel.

7. The genetically altered plant, plant part or plant cell of claim 1, wherein the N-terminal domain comprises a voltage-sensing domain, and wherein the mutation is in the voltage-sensing domain.

8. (canceled)

9. The genetically altered plant, plant part or plant cell of claim 1, wherein the stomatal voltage-gated potassium channel is a GORK channel, wherein the GORK channel comprises an amino acid sequence as defined in SEQ ID NO: 1, 3, 5 or a functional variant or homologue thereof.

10. (canceled)

11. The genetically altered plant, plant part or plant cell of claim 1, wherein the plant is a monocot or dicot.

12. The genetically altered plant part of claim 1, wherein the plant part is a seed.

13. A method of increasing at least one of growth, yield, drought tolerance, water use efficiency and/or carbon assimilation in a plant, the method comprising introducing at least one mutation into the N-terminal domain of a stomatal voltage-gated potassium channel.

14. The method of claim 13, wherein the at least one mutation alters the pattern of charged amino acids in the N-terminal domain.

15. The method of claim 14, wherein the mutation is the substitution of at least one positively charged amino acid for a negatively charged amino acid or a neutral amino acid and/or wherein the mutation is the substitution of at least one negatively charged amino acid for a positively charged amino acid or a neutral amino acid.

16. The method of claim 13, wherein the mutation is the substitution of all positively charged amino acids for negatively charged amino acids or neutral amino acids in the N-terminal domain of the stomatal voltage-gated potassium channel.

17. The method of claim 13, wherein the mutation is the substitution of all negatively charged amino acids for positively charged amino acids or -neutral amino acids in the N-terminal domain of the stomatal voltage-gated potassium channel.

18. The method of claim 13, wherein the mutation is not the substitution of all positively charged amino acids for negatively charged amino acids or neutral amino acids and the substitution of all negatively charged amino acids for positively charged amino acids or -neutral amino acids in the N-terminal domain of the stomatal voltage-gated potassium channel.

19. The method of any of claim 13, wherein the N-terminal domain comprises a voltage-sensing domain, and wherein the mutation is in the voltage-sensing domain.

20. The method of any of claim 13, the mutation increases the rate of stomatal opening and closing compared in a plant compared to the rate of stomatal opening and closing in a wild-type or control plant.

21. The method of any of claim 13, wherein the stomatal voltage-gated potassium channel is a GORK channel, wherein the GORK channel comprises an amino acid sequence as defined in SEQ ID NO: 31 to 43 or a functional variant or homologue thereof.

22. (canceled)

23. The method of any of claim 13, wherein the plant is a monocot or dicot.


SEQUENCE LISTING (55 sequences)

SEQ ID NO: 1 - protein, 820 aa, Arabidopsis thaliana
MGRLRRRQEIIDHEEEESNDDVSSRRGKLSLAETFRWLDSSEHRRIETDGHNDYKYIIHPKNRWYKAWEMFILVWAIYSSLFTPMEFGFFRGLPERLFVLDIVGQIAFLVDIVLQFFVAYRDTQSYRTVYKPTRIAFRYLKSHFLMDFIGCFPWDLIYKASGKHELVRYLLWIRLFRVRKVVEFFQRLEKDTRINYLFTRILKLLFVEVYCTHTAACIFYYLATTLPPEQEGYTWIGSLKLGDYSYENFREIDLWKRYTTALYFAIVTMATVGYGDIHAVNLREMIFVMIYVSFDMVLGAYLIGNVTALIVKGSNTERFRDKMNDLISFMNRKKLGRDLRSQITGHVRLQYDSHYTDTVMLQDIPASIRAKIAQLLYLPYIKKVPLFKGCSTEFINQIVIRLHEEYFLPGEVITEQGNVVDHLYFVCEGLLEALVTKTDGSEESVTLLGPHTSFGDISIICNISQPFTVRVCELCHLLRLDKQSFSNILEIYFHDGRTILNNIMEEKESNDRIKKLESDIVIHIGKQEAELALKVNSAAFQGDFYQLKSLIRSGADPNKTDYDGRSPLHLAACRGYEDITLFLIQEGVDVNLKDKFGHTPLFEAVKAGQEGVIGLLVKEGASFNLEDSGNFLCTTVAKGDSDFLKRLLSSGMNPNSEDYDHRTPLHVAASEGLFLMAKMLVEAGASVISKDRWGNSPLDEARLCGNKKLIKLLEDVKNAQSSIYPSSLRELQEERIERRKCTVFPFHPQEAKEERSRKHGVVVWVPSNLEKLIVTAAKELGLSDGASFVLLSEDQGRITDIDMISDGHKLYMISDTTDQT

SEQ ID NO: 2 - RNA, 2463 nt, Arabidopsis thaliana
  source 1..2463: mol_type = mRNA
atgggacgtctccggagacggcaagagataatagatcatgaagaagaggaatcaaacgacgacgtttcatcaagaagaggaaaactcagtttagccgagacgtttcggtggcttgattcatcagagcatcggagaattgaaaccgatggtcataatgattataaatacatcattcatcccaaaaacaggtggtacaaggcatgggaaatgtttatattggtgtgggcaatatactcctcattgttcactcccatggagtttggtttcttccgcggtctgcctgagagactctttgtacttgacattgttggtcagatcgcgttcttggtcgatattgttcttcagttctttgttgcctatcgcgatactcagtcctaccggactgtctacaaaccaacacgtattgctttccggtacttgaagtcgcattttctcatggatttcatcggttgcttcccttgggatcttatttataaggcatcagggaaacatgagttggtgaggtacttgttgtggataaggctatttcgggttcgcaaagtggttgagtttttccaaaggcttgagaaagacacaagaatcaactatctattcactagaatcttaaagctcttgttcgttgaagtttattgtactcacactgctgcttgtatcttctattacttggccaccactcttcctccagaacaagaagggtacacgtggatcggtagcttgaagctaggagactatagctacgagaatttccgagaaatcgatctatggaaacgttatactactgctctatacttcgccattgtcactatggcaactgtcggttatggagacattcacgcggtgaatctgagggaaatgatatttgtaatgatatatgtttcatttgatatggttctcggtgcttaccttattggtaacgtcactgccttgattgtgaaaggttcaaacacagagaggttcagagataaaatgaatgatctcataagtttcatgaaccgcaaaaaactcgggagagaccttcgtagccagataactggtcatgttagattgcagtacgacagtcactacaccgacactgtcatgcttcaggacatcccagcatcaatccgcgccaagattgcgcaattattgtatctgccttacatcaaaaaagttcctctcttcaaaggctgctccacagagtttatcaatcaaatagttataaggctccatgaagagtattttcttccaggagaagtaataacagagcaaggaaacgtcgtggatcatttgtatttcgtctgtgaaggcttactggaggctcttgttacaaaaacagatggatcagaagagagtgtgacgttacttgggcctcacacttcttttggagacatctccatcatttgcaacatttctcaacctttcactgttagggtttgtgagctatgccatcttttacgactcgataaacagtctttctcaaacatcctcgagatttattttcacgacggacgcacaatcttgaacaatattatggaggagaaggaatcaaatgataggataaagaagctagaatctgacatagtgattcacattgggaaacaagaagcagaacttgcattgaaagtaaacagtgcagctttccaaggagatttttaccagcttaagagcttaatccgatctggagccgatcctaacaaaaccgattacgatggaagatcaccgcttcatcttgcagcatgtagaggctatgaagacattacattattccttattcaggaaggtgttgatgtcaatctaaaagataagttcggacacacaccattgtttgaggctgtgaaagcaggacaagaaggagtgattggtttgcttgtcaaagaaggagcctcctttaatttagaagattcaggaaacttcctttgcacgacagttgctaaaggcgactctgattttctcaagagattgctttcaagcggtatgaacccaaacagtgaagattatgatcacagaacgccgcttcatgtcgcggcttctgaagggttattcttgatggctaaaatgttggttgaagctggagcaagcgttatttctaaagaccgatgggggaattctccgcttgatgaagcccgattgtgcggaaacaagaaactgattaagttactcgaagatgtgaaaaatgctcagtcgtctatctacccgtcaagcttgcgtgaattacaagaggagagaattgagagacggaaatgcacggtgtttccattccacccgcaagaggcgaaagaagagcgtagtagaaagcacggagttgtggtttgggtcccaagcaatctcgagaaactcatagtaaccgctgcgaaagagctagggctatcggatggagcctcatttgtactattatcagaagaccaaggtcgtatcacagacattgatatgattagtgatggacacaaattgtatatgatcagtgatactactgatcaaacataa

SEQ ID NO: 3 - protein, 818 aa, Brassica napus
MGCLRRRQESIAEEDDINDDVSRRRGGFSLAESFRWLDSPEHLKDDSDGPNEYPWIIKPSISRWYKAWELFILVWAIYSSLFTPMEFGFFRGLPENLFILDIVGQIAFLVDIVLQFFVAFQDKHNYRIDSKPTHIALRYLKSHFFLDLVSCFPWDLIYKASGKHEVVRYILWIRLFRVRKVIEFFQRLEKDTRINYLFTRILKLIFVEVYCTHTAACIFYYLATTLPAENEGYTWIGSLKLGDYSYENFRKIDIWKRYTTSLYFAIVTMATVGYGDIHAVNLREMIFVMIYVSFDMVLGAYLIGNITALIVKGSNTERFRDKMNDLASFMNRKKLRGDIRSQITHHVRLQYDSKFTNTVMLQDIPASIRAKIAQLLYTPYIEKIPLFKGCSSEFINQIVVRLHEEYFFPGEVITEQGNVVDHLYFVCEGSLEALETKTDGTEDLVELLEPHTSFGDISIICNISQPFTIRVRSLCHLLRLDKQSFSNILEIYFHDGRKILNNLMEGKESNERIKKLESDIMIHIGKQEAELALKVNSAAFQGDIYQLKSLVRSGADPNKTDYDGRAPLHLAASRGYEDITLFLIQEGVDINQKDKFGNTPLLEAVKAGQDRVIDLLVKEGASFDLEDAGNFLCTVVVKGDSDFLKRLLSSGMDPNTEDYDHRTPLHVAASEGLFLMAKMLVEAGASVVAKDRWGNSPLDEARMCGNKKLIKLLEDADTAQPYIRPSSFHEPQDEKFERRKCTVFPFHPHEEPSRKHGVMVWLPRDLQKLVETAAQELGISNEVSFVILAEEGGRITDIDMISDGQKLYLISDSTDQST

SEQ ID NO: 4 - RNA, 2454 nt, Brassica napus
  source 1..2454: mol_type = mRNA
atggggtgtctccggcgacggcaagagagtatagccgaggaagatgatataaacgacgacgtttcaaggagaagaggtgggttcagcttggccgagtcgttccggtggcttgattcccctgagcatctgaaagatgattccgatggccctaacgaatacccttggatcattaagccctcaattaggtggtacaaggcatgggagttattcatattggtctgggcaatatactcgtctttgttcactcccatggagtttggtttcttccgcggtctacccgaaaacctcttcatactcgacattgttggtcagatcgcatttttggtcgatattgttctacagttttttgtcgccttccaagataagcataactaccgaattgacagcaaaccaacacatattgctctccggtacttgaaatcgcattttttcttggatttggtcagttgcttcccatgggatctcatctataaggcttcagggaaacatgaggtggtgaggtacatattgtggataaggctgtttcgtgtgcgcaaagtgattgagttcttccaaaggcttgagaaagacacgagaatcaactatctcttcaccagaatcttgaagctcattttcgttgaggtttactgtactcacactgctgcatgcatcttctattacttggccaccactcttcctgctgagaacgaaggttatacttggatcggtagcctgaagttaggagactatagctacgagaatttccgaaagatcgacatctggaaacgttacaccacatctctctacttcgccattgtcactatggctactgtcggttatggagacatacatgccgtaaatttaagggaaatgatatttgtaatgatatatgtttcgttcgatatggttcttggtgcgtaccttattggtaacatcactgccttgattgtgaaaggatcaaacacagagagatttagagataaaatgaatgatctggcaagtttcatgaaccgaaaaaaacttaggggagacattcgtagccagataactcatcacgttagattgcagtacgacagtaaattcaccaacactgtcatgcttcaagacatcccagcctctatccgcgccaagattgcgcagttattatacacgccttacattgagaaaatccctctgttcaagggctgctcatcagagtttattaaccagatagttgtaaggctccatgaagagtatttttttccaggagaagtaatcacagagcaaggaaacgtcgttgatcatttgtatttcgtctgtgaaggctcactggaggctcttgaaacaaaaacagatggaacagaagaccttgtggagttgcttgagcctcacacttcttttggtgatatatccatcatctgcaacatctctcaacctttcactattagagttcgttcattgtgccatcttttacgcctcgataaacagtctttctccaacatcctcgagatttattttcatgatggacgcaaaatcctaaacaatcttatggagggtaaagaatcaaatgagaggataaagaagctcgaatctgacattatgattcacattgggaaacaagaagcagaacttgcattgaaggtaaacagtgcagctttccaaggagatatctaccagcttaaaagcttagtccgctctggcgccgatcctaacaaaacagattacgatggaagagcaccgcttcatcttgcagcgtctaggggatacgaagacattacattgtttcttattcaagaaggcgtcgatataaatcaaaaagataaatttggcaatacaccattgttagaggctgtaaaagcagggcaagacagagtgatcgatttgcttgtcaaagaaggagcttcctttgatttagaagatgctggaaactttctctgcacggtagttgtgaaaggcgactctgattttcttaagagattgctctcaagcggtatggacccaaacaccgaagattatgatcatagaacaccgcttcatgtcgctgcttcagaaggcttattcttgatggctaagatgcttgttgaagctggagctagcgttgtcgctaaagaccggtggggaaactctccgcttgatgaagctcgaatgtgtggaaacaagaaattgattaagttacttgaagacgcggatacagctcagccttatatacgcccgtcaagctttcatgaaccacaagatgagaaatttgagagacggaaatgcacagtgtttccattccacccacatgaggagccgagtagaaagcatggagttatggtttggcttccgcgtgacctccagaaacttgtggaaacagctgcgcaagagctcgggatatctaatgaagtctcctttgtaatattggccgaggaaggaggtcgaatcacagacattgatatgatcagtgatggacagaaactgtatttgatcagtgatagtactgatcaatcaacctga

SEQ ID NO: 5 - protein, 817 aa, Raphanus sativus
MGCLRRPQESIAEEDDTNDDVSMRRGRFSLAESFRWLDSPEHLKNDSDCPNEHPWIINPSNRWYKAWELFILLWAIYSSLFTPMEFGFFRGLPENLFVLDIIGQIAFLVDIVIQFFVAFQDKHTYRTDDKPTHIALRYMKSHFFLDLVSCFPWDLIYKASGKNEVVRYILWIRLFRVRKVIEFFQRLEKDTRINYLFTRILKLIFVEVYCTHTAACIFYYLATTLPAENEGYTWIGSLKLGDYSYENFRKIDIWKRYTTSLYFAIVTMATVGYGDIHAVNLREMIFVMIYVSLDMVLGAYLIGNITALIVKGSNTERFRDKMNDLASFMNRKKLRGDIRSQITHHVRLQYDSKFTNTVMLQDIPASIRAKIAQLLYTPYIEKIPLFKGCSSEFINQIVIRLHEEYFFPGEVITEQGNVVDHLYFVCEGSLEALATKTDGTEELVALLEPHTSFGDISIICNISQPFTVRVRSLCHLLRLDKQSFSNILEIYFHDGRKILNNLMEGKESNERIKELEPDVMIHIGKQEAELALKVNSAAFQGDIYQLKRLVRSGADPNKTDYDGRAPLHLAASRGYEDITLFLIQEGVDINQKDKFGNTPLLEAVKAGQDRVIHVLVKEGASFDLEDAGNFLCTVVLKGDSDFLRRLLSSGMDPNTEDYDHRTPLHVAASEGLFLMAKMLVEAGARVVAKDRWGNSPLDEARICGNKKLIKLLENARTAQPSICPSSSHELQDEKFKRRKCTVFPFHPHEEPNRKHGVMVWLPRDLQKLVETAAQELRLSNEASFVILSEEGGRITDVDMISDGQKLYLISDSTDQSA

SEQ ID NO: 6 - RNA, 2454 nt, Raphanus sativus
  source 1..2454: mol_type = mRNA
atggggtgtctccggcgaccgcaagagagtatagccgaggaagacgatacaaacgacgacgtttcaatgagaagaggtagattcagcttggccgagtcgttccggtggcttgattcccccgagcatctgaaaaatgattccgattgccctaacgaacacccttggatcattaatccctcaaacaggtggtacaaggcatgggagttattcatattgttatgggcaatatattcgtctttgttcactcccatggagtttggtttcttccgcggtttacccgaaaacctcttcgtactcgacattattggtcagattgcatttttggtcgatattgttatacagtttttcgttgccttccaagataaacatacctaccggactgacgacaaaccaacacatattgctctccggtacatgaaatcgcattttttcttggatttggtcagttgcttcccatgggatctcatctataaggcgtcagggaaaaacgaggtggtgaggtacatattgtggataaggctgtttcgtgtgcgcaaagtgatagagttcttccaaaggcttgagaaagacacgagaatcaactatctgttcaccagaatcttgaagctcatttttgttgaggtttattgtactcacactgctgcatgcatcttctattacttggccaccactcttcctgccgagaacgaaggttacacttggatcggtagcctgaagttaggagactatagctacgagaatttccgaaagatcgacatttggaaacgttataccacatctctctacttcgccattgtcactatggctactgtcggttatggagacatacacgcggtaaatttaagggaaatgatattcgtaatgatatatgtttcactcgatatggttctgggtgcgtaccttattggtaacatcactgccttgatagtgaaaggatcaaacacagagagatttagagataaaatgaatgatctggcaagtttcatgaaccgaaaaaaactacggggagacattcgaagccagataactcatcacgttagattgcaatacgacagtaaattcaccaacactgtcatgcttcaagacatcccagcctctatccgcgccaagattgcccaattattatacacgccttacattgagaaaatccctctgttcaagggctgttcatcagagtttattaaccagatagttataaggctccacgaagagtatttttttccgggagaagtaatcacagagcaaggaaacgttgttgatcatttgtatttcgtctgtgaaggctcactggaggctcttgctacaaaaacagatggaacagaagagcttgtggcgttgcttgagcctcacacttcttttggtgatatctccatcatttgcaacatctctcaacctttcactgttagagttcgttcattatgccatcttttacgcctggataaacagtctttctccaacatcctcgagatttattttcatgatggacgcaaaattctaaacaatcttatggagggtaaggaatcaaatgagaggataaaggagctagaacctgatgttatgattcacattgggaaacaagaagcagaacttgcactgaaggtaaacagtgcagctttccaaggagatatctaccagcttaaaagattagtccgctctggagccgatcctaacaaaacagattacgatggaagagcaccgcttcatcttgcagcgtctaggggatacgaagacattacattgtttcttattcaagaaggcgtcgatataaatcaaaaagataaatttgggaatacaccattgttagaggctgtaaaagcagggcaagacagagtgatccatgtgcttgtcaaagaaggagcttcctttgatttagaagatgctggaaactttctttgcacggtcgttctgaaaggcgactctgattttcttaggagattgctctcaagcggtatggacccaaacaccgaagattatgatcatagaacaccacttcatgtcgctgcttctgaaggattattcttgatggctaagatgcttgttgaagctggagctagagttgtcgctaaagacaggtggggaaactctcctcttgatgaagctcgaatatgtggaaacaagaaattgattaagttacttgaaaacgcgagaacagctcagccttctatatgcccgtcaagctctcatgaactacaagatgagaaatttaagagacggaaatgcacagtgtttccattccacccacacgaggagcctaatagaaagcatggagtcatggtgtggcttccgcgtgatctacagaaacttgtggaaacagctgcgcaagagctcaggctatctaatgaagcctcctttgtaatattgtcagaggaaggaggtcgaatcacagacgttgatatgatcagtgatggacagaaactgtatttgatcagtgatagtactgatcaatcagcgtga

SEQ ID NO: 7 - protein, 814 aa, Elaeis guineensis
MSPRAKNGSPEIEYELEEPIASSRGIRLFLLTSEFALGPLRRRRATSQEKLLERFVIEPDNRWYQLWTRFILVWALYSSFFTPIEFGFFRGLPNNLFWLDFAGQVAFLVDIFVQFLVAYRDSHTYRMIYEPTSIAVRYAKSSFVFDLLGCFPWDAIYRACGRKEEVRYLLWIRLTRVRKVTDFFQKMEKDIRINYLFTRIVKLIVVELYCTHTAACIFYYLATTLPASMEGYTWIGSLKLGDYSYSHFRDMDIAKRYITSLYFAIVTMATVGYGDIHAVNPREMVFIMIYVSFDMILGAYLIGNMTALIVKGSKTERFRDRMKELIKYMNRNKLGKDIRDQIKGHVRLQYESSYTEASVLQDIPVSIRAKISQTLYKPYIENVPLFKGCSAEFIHQIIIKLQEEFFLPGEVILEQGNAVDQLYFVCHGKLEGVGIGEDGPLELEPNSSFGEIAILCNIPQPYTVRVCELCRLLRIDKQIFTNILQIYFVDGRKILSNLLEGNDSIQIKQLEGDIIFHIGKQEAELALRVNNAAFYGDLHYLKGLIQAGADPNKTDYDGRSPLHLAAAKGHEDITLFLIQEGVNINLSDNFGNTPLFEAVKVGHDRVASLLFSRGAQLNLKDAGSHLCTAAAKGDSDFIRRALSYGVDPNSKDYDHRTALHIAASEGLYFIAKLLLEAGASVFAMDRWGATPLDEGRKSGNQSLMLLLEGAKSDELSKFPEHAREVQDKMHPRRCTVFPFHPWDPKEARREGVVLWIPCTIEELIRSSREQLKSSGSCILSEDGGRILDVDMISDGQKLYLVADQETTAHNNQHT

SEQ ID NO: 8 - RNA, 2426 nt, Elaeis guineensis
  source 1..2426: mol_type = mRNA
atgggtccccggagatcgagtacgagttggaggagccgattgcgtcgtcgcgggggatccgccttttcctgctcacctccgaattcgccctcggccccctccgccgccgccgggcgaccagccaggagaagcttctcgagcgcttcgtcatcgaacccgataaccggtggtaccagctgtggacgaggtttatactggtatgggcattgtactcgtccttcttcacgccgatagagttcggcttcttcagggggctcccgaacaacctcttctggctcgatttcgccggccaggtggccttcctcgtcgacatcttcgtccagttcctcgtcgcctaccgtgactctcacacctaccgcatgatctacgagcccacctccattgctgtccggtacgcgaagtcgagcttcgtcttcgacttacttggctgctttccttgggatgctatctatagggcatgcggacgtaaagaggaagtgaggtaccttttatggattcgattaactcgggtgcgcaaagtcacagatttttttcagaagatggagaaagatatccgcatcaactacttgtttacaagaattgttaaactcatagttgtggagctctactgcacgcatacagctgcctgtatattttactatcttgcaacaactttgccagcatccatggaaggctatacatggatagggagcttaaagttgggtgattatagctattcgcacttcagggatatggatatcgcaaagcgttacataacatcgttatattttgccattgtcacaatggcaactgttggttatggagacatacatgcagttaatcccagggagatggtattcatcatgatttatgtttcttttgatatgattctgggagcttaccttattggcaacatgactgcattgattgtgaaaggatcaaagacagaaaggtttagggacagaatgaaagaattaattaaatacatgaacagaaacaaacttggaaaggatattcgtgaccagatcaaaggacatgtacgcttacaatatgaaagcagctacacagaggcttcagttcttcaggatatcccagtttcaattcgtgctaagatatcacagacactatacaaaccgtatatcgaaaatgttcccctgtttaagggatgctcagccgaattcattcaccagattattatcaagctgcaggaagagtttttccttccaggggaggtgatattggaacagggaaatgcagtagatcagctatattttgtctgccatggtaagctggaaggagtaggaattggtgaagatggacctttggaactggagcccaacagttcctttggagaaattgcgattctgtgcaacattcctcagccttacactgttcgtgtttgtgaattgtgtagacttttgcgaattgataaacaaatcttcacaaatattctacaaatttactttgttgatggtcggaaaattttgagcaaccttctagagggtaatgattccattcaaattaagcaactagaaggtgatattatattccatatcggtaagcaagaagccgagcttgctttaagagtaaacaatgctgctttctatggagatctccattaccttaaaggtttgatccaagcaggagcagatcctaataagactgattatgatggacgctcacctttgcatctcgcagcagcaaaagggcatgaagatatcactctatttctcattcaagaaggcgtgaacattaacctttctgacaactttggaaatacacccttgtttgaggcagtcaaggttggacatgatcgagtggcttctttactctttagtagaggtgcacaattaaatcttaaagatgctggtagccacctttgtacagctgctgcaaagggggattcagattttattagaagagctttatcttacggtgttgatccaaactcaaaggactacgatcaccgcacagcgctgcatatagctgcttctgagggattatattttatagcaaagttacttttagaggctggggcaagtgtttttgctatggacagatggggagctacaccattggatgaaggacgaaagagtggaaaccaatccttgatgctgctgttagaaggtgcaaagtctgacgagctgtccaagtttcctgaacatgctcgggaagttcaagataaaatgcatccaaggaggtgcacagtgtttccttttcacccgtgggatccgaaagaagcaagaagagagggagtcgtgctatggatcccatgcaccatagaggagctcattagatcctctagagagcagctaaagtcttcaggttcatgcatactatcagaagatgggggcaggattcttgatgtagatatgatatctgatggccagaaattatatctagttgcagatcaagaaacaactgcacataataaccagcatacttga

SEQ ID NO: 9 - protein, 858 aa, Oryza sativa
MGRGIGSKRRVEDDDGENMPGRKKKEEEEEEEDDDGEEEYEVDVVRDRIGSSRGSRLALFGSDLRLGRFRPRRRRVAPVDGDDGIFQDFVIDPDNKWYRLWTRFILVWAVYSSFFTPLEFGFFRGLPRNLFFLDIAGQIAFLIDIVLRFFVAYRDPDTYRMVHNPTSIALRYCKSSFIFDLLGCFPWDAIYKACGSKEEVRYLLWIRLTRAMKVTEFFRSMEKDIRINYLFTRIVKLIVVELYCTHTAACIFYYLATTLPESMEGYTWIGSLQLGDYSYSHFREIDLTKRYMTSLYFAIVTMATVGYGDIHAVNVREMIFIMIYVSFDMILGAYLIGNMTALIVKGSRTERFRDKMKEVIRYMNRNKLGKDIREQIKGHLRLQYESSYTEASVLQDIPVSIRAKISQTLYKPYIESIPLFKGCSAEFIQQIVIRLQEEFFLPGEVILEQGSAVDQLYFVCHGALEGVGIGEDGQEETILMLEPESSFGEIAVLCNIPQPFTVRVCELCRLLRLDKQSFTNILEIFFVDGRRILSNLSESSEYGSRIKQLESDITFHIGKQEAELTLRVNNAAFYGDMHQLKSLIRAGADPKNTDYDGRSPLHLAACKGFEDVVQFLLHEGVDIDLSDKFGNTPLLEAVKQGHDRVATLLFSKGAKLSLENAGSHLCTAVARGDTDFVRRALAYGGDPNARDYDHRAPLHIAAAEGLYLMAKLLVDAGASVFATDRWGTTPLDEGRRCGSRTMVQLLEAAKSGELSRYPERGEEVRDKMHPRRCSVFPHHPWDGGERRREGVVVWIPHTIEGLVSSAQEKLGLAGSGEGLRLLGEDGARVLDVDMVHDGQKLYLVVGGGGDDGGTEARQ

SEQ ID NO: 10 - RNA, 2577 nt, Oryza sativa
  source 1..2577: mol_type = mRNA
atggggagggggatagggtcgaagcggagggtggaggacgacgacggggagaatatgccggggaggaagaagaaggaggaggaggaggaggaggaggatgacgatggggaagaggagtacgaggtggacgtggtgcgcgaccggatcgggtcgtcgcgggggagccgcctggcgctgttcggctccgacctccgcctcggccgcttccgcccgcgccgccgccgcgtcgcccccgtcgacggcgacgatggcatcttccaggacttcgtcatcgaccccgacaacaagtggtatcggttatggacgaggttcatactagtgtgggcagtgtacagttccttcttcacgccattggaatttggcttcttcagggggctccccaggaacctcttcttcctagacatagctggtcagatcgccttcctcatcgacattgttctcaggttcttcgtggcctaccgtgatcctgacacctaccgcatggttcacaatcccacctccatcgctctccgatattgtaaatcaagcttcatttttgatcttcttggttgttttccgtgggatgctatctataaggcttgtggaagtaaagaagaagtaagatacctattgtggattcggttgacacgagctatgaaggtgacagagttcttcaggagcatggaaaaagatatccgtataaattacctgtttactaggattgtgaaactcatagttgtggaactctactgtacgcacacagcagcatgtatcttctactatcttgcaacaacacttcctgaatcaatggaaggatatacatggatagggagcttgcagttgggagactatagttattctcatttcagggagattgatcttaccaagcgttatatgacatcgctgtactttgccatagtcaccatggcaactgttggttatggtgacattcatgctgtgaatgtcagggagatgatattcatcatgatttatgtttcctttgatatgattcttggagcctacctcattggtaacatgactgcacttattgtcaagggctcgagaaccgagcgattcagggataagatgaaagaagttatcaggtacatgaacagaaacaagcttggaaaggacataagggaacagattaaaggacatttgaggttgcagtatgaaagcagctacactgaagcttctgtccttcaggatatcccagtctcaattcgtgcgaagatttcacaaacactgtataagccatatatcgaaagcattccactgtttaaaggatgttcagcagaattcattcaacagattgtgatcaggctgcaggaagagttcttcctacccggagaggttattttggagcagggaagtgcagttgatcaactgtactttgtctgtcatggggcactggaaggtgttggcattggtgaagatggtcaagaagagaccattctaatgctagagcctgaaagttcttttggagaaatcgctgttctctgcaatatcccacaacccttcaccgttcgtgtctgcgaactttgtcgtcttttgcggcttgacaagcaatccttcacaaacatcctggagatcttcttcgttgatggaagaagaattctgagcaacctttctgagagtagcgaatacggcagccggatcaagcagctggaatccgacatcacgttccacatcgggaagcaagaggcagagctgacgctgcgagtgaacaacgctgcgttctacggtgacatgcatcagctcaagagcctgatccgtgcaggagccgatcccaagaacaccgattacgacgggcgatctcctctgcatcttgcggcttgcaaagggttcgaagatgtcgtccagttcctcctccacgaaggcgtcgacatcgatctttccgacaaatttggcaacacgccattgctggaggcggtgaagcaggggcacgaccgggtggcgacgctgctgttcagcaagggggcgaagctgagcctggagaacgccgggagccacctgtgcacggcggtggcgaggggcgacacggacttcgtccggcgggcgctcgcctacggcggcgacccgaacgcgagggactacgaccaccgcgcgccgctccacatcgccgccgccgagggcctctacctgatggcgaagctgctcgtcgacgccggcgccagcgtgttcgccaccgaccggtggggcacgacgccgctggacgaagggcgcaggtgcgggagccggacgatggtgcagctgctggaggcggccaagtccggcgagctctccaggtacccggagcgcggcgaggaggtgcgggacaagatgcacccgcggcggtgctccgtgttcccgcaccacccgtgggacggcggcgagcggcggcgggaaggggtggtggtgtggatcccgcacaccatcgaggggctcgtcagctcggcgcaggagaagctcggcctcgccggctccggcgaggggctccggctgctcggcgaggacggcgccagagtgctcgacgtcgacatggtccacgacgggcagaagctctacctcgtcgtcggcggcggcggcgatgacggcggcacggaggcaaggcagtga

SEQ ID NO: 11 - protein, 828 aa, Arabidopsis thaliana
MGGSSGGGVSYRSGGESDVELEDYEVDDFRDGIVESRGNRFNPLTNFLGLDFAGGSGGKFTVINGIRDISRGSIVHPDNRWYKAWTMFILIWALYSSFFTPLEFGFFRGLPENLFILDIAGQIAFLVDIVLTFFVAYRDSRTYRMIYKRSSIALRYLKSTFIIDLLACMPWDIIYKAAGEKEEVRYLLLIRLYRVHRVILFFHKMEKDIRINYLFTRIVKLIFVELYCTHTAACIFYYLATTLPASQEGYTWIGSLKLGDYSYSKFREIDLWTRYTTSMYFAVVTMATVGYGDIHAVNMREMIFAMVYISFDMILGAYLIGNMTALIVKGSKTERFRDKMADIMRYMNRNKLGRNIRGQITGHLRLQYESSYTEAAVLQDIPVSIRAKIAQTLYLPYIEKVPLFRGCSSEFINQIVIRLHEEFFLPGEVIMEQGSVVDQLYFVCHGVLEEIGITKDGSEEIVAVLQPDHSFGEISILCNIPQPYTVRVAELCRILRLDKQSFMNILEIFFHDGRRILNNLLEGKESNVRIKQLESDITFHISKQEAELALKLNSAAFYGDLYQLKSLIRAGGDPNKTDYDGRSPLHLAASRGYEDITLYLIQESVDVNIKDKLGSTPLLEAIKNGNDRVAALLVKEGATLNIENAGTFLCTVVAKGDSDFLKRLLSNGIDPNSKDYDHRTPLHVAASEGFYVLAIQLVEASANVLAKDRWGNTPLDEALGCGNKMLIKLLEDAKNSQISSFPSGSKEPKDKVYKKKCTVYFSHPGDSKEKRRRGIVLWVPRSIEELIRTAKEQLNVPEASCVLSEDEAKIIDVDLISDGQKLYLAVET

SEQ ID NO: 12 - RNA, 2487 nt, Arabidopsis thaliana
  source 1..2487: mol_type = mRNA
atgggaggtagtagcggcggcggagtttcttatcgtagcggcggcgaatcagacgtggaattagaggattacgaggttgatgatttcagagatgggattgtagaatcgcgaggaaacagattcaatcctctcaccaatttcttagggttagacttcgccggcggtagcggtggaaagttcaccgtcattaatggaatcagagatatctccagaggctccattgttcatcccgataaccggtggtacaaggcgtggacgatgtttatattgatatgggcactttattcttccttcttcactccattggaattcggattcttcaggggattaccagagaatctgttcatcctcgatatcgctggccaaatcgctttcttagtagatattgtcttgacattcttcgttgcttatcgtgatagccgaacttatagaatgatctataaacgcagctcaattgctttacggtacttaaaatcaacttttataattgacttacttgcttgcatgccatgggatatcatctacaaggctgcaggcgaaaaagaagaagtgagatacctattgttgataaggctatatcgagttcatagagtaatcctgtttttccacaaaatggagaaagatataagaatcaattacctttttacaagaatcgtcaagcttatattcgtcgagctttattgcactcacaccgcagcttgtatcttctattacttggccacgacgcttcctgcttctcaagaagggtacacttggattggaagcttgaagttaggagattacagttactcgaagtttagagagatcgatctctggactcgatacactacttctatgtactttgcagttgttactatggcaactgttggttatggagatatacacgcggttaatatgcgggaaatgatattcgcgatggtctacatttcattcgatatgattctaggagcttacttgattggtaacatgacagctttgattgtaaaaggttcgaaaacagagagattccgggataagatggctgatatcatgcggtatatgaaccgaaacaaactcggtaggaacatccgtggtcagattaccggacatttgcggttgcagtacgaaagcagctacactgaagcagctgttcttcaagacattcctgtctctatccgtgctaagattgcgcaaactttatacttgccgtatattgagaaagttcctctctttcgcggatgctcatctgaattcattaaccagattgttataagacttcatgaagagttctttcttcctggagaagttataatggagcaaggaagcgtcgttgatcagctctactttgtttgtcacggtgtattggaggagataggtataacaaaggatggatctgaagaaatagtggcagttctacaaccagatcattcttttggagagatttcaatcctctgcaatattcctcagccttacacggttagagttgcagagctatgtcggattctaaggcttgacaagcaatctttcatgaacatactcgagatatttttccatgatggacggaggattctcaacaatctgcttgaagggaaagaatctaatgtccggattaagcaactagaatccgatataacctttcatatcagtaaacaagaggcagagctagctctgaagttgaatagtgcggctttctacggtgatctttatcagcttaagagtttgattcgagctggaggtgacccgaataagacagattatgacggaagatcgcctttgcatcttgcagcctctagaggatatgaagatattacattatatcttatacaagaatcagttgatgtaaatatcaaagataaattggggagcacacctttactagaagcaatcaagaatgggaatgatcgtgttgcggcgttgcttgttaaagaaggagctacgttgaacatagagaacgcagggactttcctttgcaccgtggttgcgaaaggagacagcgatttcttgaaacggcttcttagtaacggaattgatcctaattccaaagattatgatcacagaacacctcttcatgtagctgcctctgaaggattttatgtcttggcgatccaattggtagaagcaagtgctaatgttcttgcaaaagacagatggggaaacacacctcttgatgaagctttgggttgtgggaacaagatgttgataaagttactcgaagacgctaagaattctcaaatctcttcgtttccgagtggctccaaagagcctaaagataaagtctataagaagaaatgtacagtgtatttttcacatcctggtgattcgaaagagaaaagaaggcgcgggattgtactgtgggtacctcgaagcatcgaggagcttataagaacagcaaaggagcagctgaatgttccggaggcttcttgtgtattatctgaagatgaagccaaaattattgatgtagatttgataagtgatggacaaaaactgtatttggctgttgaaacataa

SEQ ID NO: 13 - protein, 829 aa, Brassica napus
MGGSSGGGVSYRSEVDSDVELEDYEVDDDFGEGIVESRGNRFNPLTNFLGLDFTGGNGGKFTVINGIRDISRGSVVHPDSGCYKAWTMFIVIWALYSSFFTPLEFGFFRGLPGNLFILDILGQIAFLVDIVLTFFVAYRDSRTYRMVYRRSSIALRYLKSTFIIDFLSCMPWDIIYKVAGRKEEVRYLLLIRLYRVRRVILFFHKMEKDIRINYLFTRIVKLIFVELYCTHTAACIFYYLATTLPASQEGYTWIGSLKLGDYSYAKFREIDLWTRYTTSMYFAVVTMATVGYGDIHAVNMREMIFAMAYISFDMILGAYLIGNMTALIVKGSKTERFRDKMADIMRYMNRNKLGRNIRGQITGHLRLQYESSYTEAAVLQDIPVSIRAKIAQTLYLSYIEKVPLFRGCSSEFINQIVIRLHEEFFLPGEVIMEQGSVVDQLYFVCHGVLEEIGTAKDGSEEIVSLLQPDNSFGEISILCNIPQPYTVRVSELCRVLRLDKQSFMNILEIYFHDGRRILNNLLEGKESNGRIKQLESDITFHISKQEAELALKLNSAAFYGDLYQLKSLIRAGADPNKTDYDGRSPLHLAASRGYEDITLYLIQESVDVNIKDKLGNTPLLEAIKNGNDRVAALLVKEGATLSIENAGTFLCTVVAKGDSDFLKRLLNNGIDPNSKDYDQRTPLHVASSEGLYLLARQLVEAGANVLKKDRWGNTPLDEALVCGNKMLIKLLEDAKTSQMSTFLNSSKEIKDKVYKKKCTVYSSHPNDSKEKRRRGIVLWVPKSIEELVRSAAEQLNFPEASCVLSEDEGKIIDVELISDGQKLYLTIET

SEQ ID NO: 14 - RNA, 2490 nt, Brassica napus
  source 1..2490: mol_type = mRNA
atgggaggtagcagcggcggcggagtttcgtaccggagcgaagtcgactcggacgtggagttggaggattacgaggtggatgatgatttcggagaaggcatcgtagaatcgcgaggaaacagattcaatcccctcaccaatttcttaggattagatttcaccggaggtaacggcggaaagttcaccgtcattaacggaatcagagatatctccagaggctccgttgttcatcctgatagcggatgctacaaggcgtggaccatgttcatagtgatatgggcactgtactcttccttcttcactccattggagttcggattcttcaggggattaccagggaatctgttcatcctcgacatccttggccaaatcgctttcttagtcgatattgtgttaacattcttcgtggcgtatagagacagcagaacttacagaatggtctatagacgcagctccatcgctttacgctacttaaaatcaacttttattattgattttctatcatgtatgccatgggatatcatctacaaggtagcaggtcgaaaagaagaagtgagatacctattgttgataaggctatatcgagttcgaagagtgatcctgtttttccacaaaatggagaaagacataagaatcaattatcttttcacaagaatcgtcaagcttatattcgtcgagctctattgcactcacaccgctgcttgcatcttctattacttggccaccacgcttcctgcttctcaagaagggtacacttggattggaagcttgaaactgggggattacagttacgctaagtttagagagatcgatctctggacccgttacaccacttctatgtacttcgctgttgtcactatggcaactgttggttatggagatatacatgcggttaacatgcgggaaatgatattcgcgatggcctacatatcattcgacatgattctaggtgcttacttgatcggtaacatgacagctttgatagtaaaaggctcaaaaacagagagattcagggacaagatggcggatattatgaggtatatgaaccgaaacaaactcggtagaaacatccgtggtcagatcactggtcatttgcggttgcagtacgaaagtagttacactgaagcagctgttcttcaagacatacctgtctctatccgcgctaagattgctcaaacgttatatttgtcgtatattgagaaagtccctctcttccgtggatgctcatctgaattcattaaccagattgttataagacttcatgaagagttttttctccctggagaagtaataatggagcaaggaagtgttgttgatcaactctacttcgtctgtcatggtgtattggaggagataggtacagctaaagatggatctgaagagatagtatcactacttcaaccagataactcctttggagagatttcaatcctctgcaacattcctcagccttacacagttcgagtttccgagctgtgtagggttctaaggctagacaaacaatcttttatgaacatactcgagatatatttccacgatggacggaggatactcaacaatctgcttgaagggaaagaatctaacggcaggattaagcagctggaatctgatattacctttcatatcagtaaacaagaggcagagctagctttgaagttgaatagtgctgctttctatggcgatctttatcagcttaagagcttgattcgagctggagctgacccaaataaaacagattatgatggaagatcgcctttgcatcttgcagcgtctagaggatatgaagacatcacactgtatcttattcaagaatcggtagacgtaaatatcaaagataaactggggaacacgccgttactagaagcaatcaagaacgggaacgatcgtgttgcggcgttgcttgtgaaagaaggtgcaacgttgagcatagagaacgcagggacttttctttgcactgtggttgccaaaggagacagcgatttcttaaaacgtcttctcaacaatggcattgatccaaactctaaagattatgatcagaggacacctcttcacgttgcttcctctgaagggttgtatctcttggcaagacagttggtagaagcaggtgctaatgttcttaaaaaggacaggtggggaaatactcctctagatgaagccctagtttgtggaaacaagatgttgataaaactacttgaagacgctaagacttctcagatgtctacgtttctgaatagctctaaagagatcaaagataaagtctataagaagaaatgcacagtgtattcttcacatccgaatgattccaaagagaaaagaagacgagggattgtattgtgggtgcctaaaagcatcgaggagcttgtaagaagtgcagcagaacagctgaattttccggaagcttcttgtgttttgtctgaagatgaaggtaaaattattgatgtagagttgataagtgatggacaaaaactgtatctgactattgaaacataa

SEQ ID NO: 15 - protein, 830 aa, Raphanus sativus
MGGSSGNGVSYRGEGESDVELEDYEVDDDDFRDGIVETRGNRFNPLTNFLGLDLAGGNGGKFTVINGIRDISRGSVIHPDNRCYKAWTTFILIWALYSSFFTPLEFGFFRGLPENLFILDIAGQIAFLVDIVLTFFVAYRDSRTYRMVYRRSSIALRYLKSSFVIDLLACMPWDIIYKAAGEKEEVRYLLLIRLYRVRRVILFFHKMEKDIRINYLFTRIVKLIFVELYCTHTAACIFYYLATTLPASQEGYTWIGSLKLGDYSYAKFREIDLWTRYTTSMYFAIVTMATVGYGDIHAVNMREMIFAMIYISFDMILGAYLIGNMTALIVKGSKTERFRDKMADIMRYMNRNKLSRNIRGQITGHLRLQYQSSYTEAAVLQDIPVSIRAKIAQTLYLPYIEKVPLFRGCSSEFINQIVIRLHEEFFLPGEVIMEQGSVVDQLYFVCHGVLEEIGTAKDGSEEIVSLLQPDNSFGEISILCNIPQPYTVRVSELCRILRLDKQSFMNILEIYFHDGRRILNNLLEGKESNVRIKELESDITFHISKQEGELALKLNSAAFYGDLYQLKSLIRAGADPNKTDYDGRSPLHLAASRGYEDITLYLIQESVDVNIKDKLGNTPLLEAIKNGNDRVAALLVKEGATLSIENAGTFLCTVVAKGDSDFLKRLLNNGIDPNSKDYDHRTPLHVAASEGLYLLAMQLVEAGANVLKKDRWGNTPLDEALGCGNKMLIKLLEDAKSSQISSFPSSSKELKDKVYKKKCTVYSSHPNDAKETRRRGIVLWVPRNIEELVRTAAEQLNVPEASCVLSEDEGKIIDVDLISDGQKLYLTVET

SEQ ID NO: 16 - RNA, 2493 nt, Raphanus sativus
  source 1..2493: mol_type = mRNA
atgggaggcagtagcggcaacggagtatcgtaccggggcgagggagaatctgacgtggagttggaggattacgaggtggatgatgatgacttcagagatgggattgtagaaacgcgaggaaacagattcaaccccctcacgaacttcctagggttagatttggccggaggtaacggaggaaagttcaccgtcattaacggaatcagagatatctccagaggctccgttattcatcccgataaccgttgctacaaggcgtggaccacgttcatactgatatgggcactttactcttccttctttactccactagaatttggattcttcaggggactaccagagaatctattcatcctagacattgcaggccaaatcgctttcttagtcgatatcgtgctaacgttcttcgtggcttatagagatagcagaacttacagaatggtctatagacgcagctccatcgctctacggtacttaaaatcatcttttgttattgatttacttgcttgcatgccatgggatatcatttacaaggctgcaggtgaaaaagaagaagtgagatacctattgttgataagactatatcgagttcgtagagtaatcctgttcttccacaaaatggagaaagacataagaatcaattaccttttcacaagaatcgtcaagcttatatttgttgagctctattgcactcacaccgcggcttgtatcttctattacctggccaccacgctacctgcttctcaagaagggtatacttggattggaagcttgaaactgggagattacagttacgctaagtttagagagatcgatctctggactcggtacacaacttctatgtactttgcaattgtcactatggcaactgttggttatggagatatacatgcggttaatatgcgggaaatgatatttgcgatgatctacatatcattcgacatgattctaggtgcttacttgatcggtaacatgacagctttgatagtaaaaggttcaaaaacagagagattcagggacaagatggcggatattatgaggtatatgaaccgaaacaaactcagtagaaacatccgtggtcagatcactggacatttacggttgcagtaccaaagtagttacaccgaggcagctgttcttcaagacatacctgtctctatccgcgccaagattgcacaaactttatacttgccatatattgagaaggttcctctcttccgtggctgctcatctgagttcattaaccagattgttataagacttcatgaagagttctttctccctggagaagttatcatggagcaaggaagcgttgtggatcagctctactttgtttgtcatggtgttttggaggagataggtacagctaaggatggatcagaagagatagtgtcacttcttcaaccagataactcttttggagagatatcaatcctctgcaacattcctcagccttacacagttcgagtttctgagctgtgtcggattctaagactagataaacaatctttcatgaacatcctcgagatatatttccatgacggaaggaggatcctcaacaatctgcttgaagggaaagaatctaatgtccggattaaggagctggaatctgatattacatttcatatcagtaaacaagagggagagctagctttgaagttgaacagtgctgctttctacggtgatctttaccagcttaagagcttgattcgagctggagctgacccgaataagacagattatgacggaagatcacctttgcatcttgcagcctctagaggatatgaagacataacattgtacctaatccaagaatcagtagatgtaaatatcaaagataaactagggaacacgccgttactagaagcaatcaagaacgggaatgatcgtgtcgcggctttgcttgtgaaagaaggtgcaacgttgagcatagagaacgcagggactttcctttgcacggtggttgctaaaggagacagcgatttcttgaaacgtcttctcaacaacggcattgatcctaactctaaagactatgatcacaggacacctctccacgttgctgcttctgaaggattgtatctattagcaatgcaactagtagaagcaggtgctaatgttcttaaaaaagacaggtgggggaatactcctttagacgaagccctaggttgtggtaataagatgttgataaaactacttgaagacgctaagagttcacagatctcttcgtttccaagtagctccaaagagctcaaagataaagtatacaagaagaaatgtacagtgtattcttcacatccgaatgacgcaaaagaaacaagaagacgtgggattgtgttgtgggtgcctagaaacatcgaggagcttgtgagaactgcagcagaacagttgaatgttcctgaagcttcttgtgtgttgtctgaagatgaaggtaaaatcattgatgtagatttgattagtgatggacaaaagctgtatctgaccgttgaaacataa

SEQ ID NO: 17 - protein, 850 aa, Glycine max
MTGKKNVKAATTTTPEEEDQRKQTTTSSSPSSSSRSSSSSSSSDEREYEVQDLRDRLKSSRGSRFDLIENQLGLNSTWSKFSRQALLHGIRGFSVDFVIHPDNRWYRAWTKFILLWAVYSSFFTPMEFGFFRGLPENLFILDIIGQIAFLVDIVLQFFVAYRDSQTYRTVYKRTPIALRYLKSNFIFDLLGCMPWDIIYKACGRKEEVRYLLWIRLYRVRKVTDFFHKLEKDIRVNYIITRIVKLIVVELYCTHTAACIFYYLATTLPESQEGYTWIGSLKLGDFSYSHFREIDLWKRYTTSLYFAIVTMATVGYGDIHAVNMREMVFIMVYVSFDMILGAYLIGNMTALIVKGSKTEKFRDKMTDLMKYMNRNRLGRDIREQIKGHVRLQYESSYTEASVIQDIPISIRAKISQTLYLPYIEKVSLFKGCSSEFIRQIVIRLHEEFFLPGEVIMEQGNVVDQLYFVCHGVLEEVGTAEDGTEETVSLLQPNSSFGEISILCNIPQPYTVRVCELSRLLRLDKQSFTNILDIYFYDGRKVLNNLLEGKESFRDKQLESDITFHIGKQEAELALKVNNAAFNGDLYQLKGLIRAGADPNKTDYDGRSPLHLAASRGYEDITLFLIQERVDVNIKDNFGNTPLLEAVKNGHDRVASLLVREGASMKIENAGSFLCTAVARGDSDYLKRLLSNGMDPNLKDYDYRSPLHIAAAEGLYFMAKLLLEGGASVFTKDRWGNTPLDEARMCGNKNLIKLLEDAKSAQLSEFPSQEYTDKMHPKKCTVFPYHPWDPKDNRRHGIVLWIPHSIQELIKSAAEQIEFSGDACILSEDAGKVTDVDMIKDGQKLYLVHETH

SEQ ID NO: 18 - RNA, 2553 nt, Glycine max
  source 1..2553: mol_type = mRNA
atgacggggaagaagaatgtgaaggcggcgacaaccacgacaccggaagaggaagaccagaggaagcaaacgacgacgtcgtcttccccctcgtcgtcttcgcgctcctcctcctcgtcgtcgtcttcggatgagagagagtacgaggtgcaggacctgcgtgaccggttgaaatcgtcgcgagggagcaggttcgatctcatagagaaccagttgggactcaactcgacttggagcaagttcagtcgccaagcgcttctccatggaatccgaggcttctccgtggacttcgttattcatcctgataacaggtggtatcgggcatggacaaaattcatattgctatgggcagtgtattcatccttcttcaccccaatggagtttgggtttttccgtggcctgcctgagaatctctttattctggacatcattggacaaatagcttttcttgtagatattgtcttgcaattttttgttgcttatagagatagtcagacctatcgtacggtctacaagagaacccctattgctctgcggtatctgaaatctaacttcatatttgatcttcttggatgtatgccctgggacatcatctacaaggcttgtggaagaaaagaggaagtgagataccttttgtggatcagactatatagggttcggaaagtcacagattttttccacaagttggagaaggacatacgtgtcaattacattattacccggattgtgaaactgatagtagttgaactctattgcacccatacagcagcctgcattttctactatttggctaccacactacctgaatcccaagaagggtacacatggataggaagtttaaaactaggtgactttagttattcacatttcagagaaattgatctttggaagcgctatacaacatctctgtacttcgctattgttacaatggctacagttggctatggagatatacatgcagtaaatatgagggagatggtattcataatggtgtatgtttcatttgatatgattcttggtgcctatttgattggtaacatgacagctttgatagtcaagggatcaaagactgaaaagtttagggacaagatgacagaccttatgaaatatatgaacagaaataggcttggaagggatatccgtgaacaaattaagggccatgtgcggttacagtatgagagtagctacactgaggcttctgtcattcaggatatccctatatctattcgagctaagatatcccaaacattatatttgccgtacattgaaaaggtttctctttttaagggatgctcttctgaattcatccgtcagattgttattaggctccatgaggagttctttcttccaggggaagttataatggaacaaggaaatgttgtggatcaactatattttgtctgccatggagtactggaggaagtaggcactgctgaagatgggactgaagaaactgtttcacttctgcagcctaacagttcattcggagaaatatctattctttgcaacattcctcaaccatatactgtccgtgtttgtgaactgagtagactcctgcgacttgataaacaatcatttacaaatatcctagacatatacttttatgatggaaggaaagtattaaacaaccttctagagggaaaagaatcttttcgagataagcagttggagtcagacatcacatttcatattggaaagcaagaagctgagcttgctttgaaggtcaataatgcagctttcaatggggatctgtatcagctaaaaggtttaattcgcgctggagctgatcccaacaagacagattatgatggaaggtcacctttgcatcttgcagcatctagaggatatgaagatatcacacttttccttattcaagaacgtgtagatgtcaatattaaagataacttcgggaacacacccttacttgaagcagttaagaatggacatgatcgggttgcttctttacttgttagagaaggggcctctatgaagattgaaaatgctggtagttttttgtgtactgcagttgcaaggggagattcagattatcttaaaagacttttatccaatggcatggatcctaacttaaaagattatgattaccgaagtcctcttcacattgctgcagctgaaggtttatatttcatggcaaagttgctattagaaggaggagctagtgtttttaccaaagacagatggggaaatacgccgcttgatgaagctcggatgtgtggaaataagaacctgatcaagcttctggaagatgcaaaatctgctcaactgtcagaattcccatcccaagaatatacagacaaaatgcatccaaagaagtgcacagtgttcccttaccacccgtgggatccgaaagataatagaagacatggaattgtattatggattccacactccatacaagagctaatcaaatcagcggcagaacaaatagaattttctggcgatgcttgtattttatcagaagatgctggtaaagttactgatgtggacatgatcaaggatggtcagaagctgtatttagtccatgaaacacattag

SEQ ID NO: 19 - protein, 824 aa, Solanum lycopersicum
MSMKRELREERNGRDSPKEYKMDDLRDSMKSLRSTSRLAMMENELIADSTPWRFSSENVLNGLRGLSQGFVIYPDDRWYKLWDKFILIWAIYSTFFTPMEFGFFKGLPRKLFLLDICGQIAFLVDIVIQFFVAYRDSQTYKMVYRRTPIALRYLKSHFILDVLSCMPWDNIYKASGRKEGVRYLLWIRLSRVRRVTDFFQKMEKDIRINYLFTRIVKLITVELYCTHTAACIFYFLATTLPEEKEGYTWIGSLTLGDYSYSHFREIDLWRRYITSLYFAIVTMATVGYGDIHAVNLREMIFVMVYVSFDMILGAYLIGNMTALIVKGSKTVRYRDKMTDLMNYMNRNRLGRDIRSQIKDHLRLQYESAYTDGAVLQDLPISIRAKISQTLYLSCIENIPLFRECSAEFISQIVTRVHEEFFLPGEVIMEQGHVVDQLYFVCDGVLEEVGIGEDGSQETVALLEPNSSFGEISILCNIPQPYTVRVSELCRLIRIDKQSFSNILEIYFHDGRRILTNLLEGKDLRVKQLESDITFHIGKQEAELALKVNSAAYHGDLHQLKSLIRAGADPNKKDYDGRSPLHLSASRGYEDISIFLIKEGVDFNASDNFGNTPLFEAIKNGHDRVASLLVKEGAFLKIENAGSFLCTLVTKGDSDLLRRLLSNGIDANSKDYDHRTPLHVAASQGLLAMARLLLGAGASVFSKDRWGNTPFDEARLSGNNQLIKLLEEAKSAQTSEIHSVSHEISEKIHLRKCTVYPIHPWEPKDLRKHGVVLWVPTSMEELVTAASEQLNFSSGSCILSEDAGKILDIDMISDGQKLYLISETT

SEQ ID NO: 20 - RNA, 2475 nt, Solanum lycopersicum
  source 1..2475: mol_type = mRNA
atgtcgatgaagagagaattacgagaagagagaaatggaagagattcaccaaaagagtataaaatggatgatttgagagatagtatgaaatcgttgcgaagtaccagtcgattggcgatgatggaaaacgagctaattgccgattcaactccttggagatttagcagtgaaaacgtcctcaatggcctcagaggcctttctcaaggtttcgtcatttatcccgatgacaggtggtacaagctatgggataagtttatcctgatttgggctatatattcaactttttttacaccaatggagtttggattcttcaaaggattgcccaggaaactatttcttttagacatttgtggtcaaatagcttttcttgtcgacatagtcatacaattttttgtagcctacagagacagtcagacatacaagatggtgtataggcgaacccctattgctctccggtacttgaaatctcattttatcttggatgtgctcagttgcatgccgtgggataacatttataaggcttctggcagaaaagaaggagtgagataccttttatggattagattaagcagggtgcggagggttactgactttttccagaagatggagaaagatattcggatcaattatctcttcactaggattgtaaagcttatcactgttgaactttattgcacgcatacagcagcttgcatcttttactttctggcaactactctgcctgaagagaaagaaggctacacatggattggtagtttgaccctgggagattacagttattcacactttagagagattgatctctggaggcggtacatcacttccctgtactttgcaattgtcactatggcaactgtgggttatggtgacatacatgcagtcaatctgagggaaatgatatttgtaatggtttatgtctcttttgatatgattcttggtgcttacttgatcggtaacatgacagcattgattgtcaagggatcaaaaactgtgcgatacagggacaaaatgacagatcttatgaactatatgaacagaaatagactcggaagggatattcgtagtcaaattaaagatcacttgcgattacaatatgaaagcgcttacactgatggagccgttctccaggaccttcccatctcaattcgtgcaaagatatcccagactttatatctgtcttgcattgaaaatattcctcttttcagggagtgctccgcagaattcataagtcaaattgtaactcgagtgcatgaagaatttttcctcccaggagaagtgatcatggaacaggggcatgtagtggaccaactttattttgtctgtgatggtgtgctggaagaagttggtataggagaagatgggtcacaagagacagtagcacttcttgaacctaatagctcgtttggagaaatatccattctttgcaacattccacagccatatactgtccgtgttagtgaactatgcagactcatacggattgataagcaatcattttcaaatattttggagatctattttcatgatggaagaagaatcttgacgaacttactagagggaaaggatcttcgtgtgaagcaactggagtcagatataacattccatattgggaaacaagaggctgagcttgctttgaaagtgaatagtgcagcttatcatggtgatttgcaccagctgaagagtctgattcgagctggagctgatcccaacaagaaagattatgatggaagatcacctctgcatctttcagcatccagaggatatgaagacatctctattttccttatcaaagaaggtgtcgatttcaatgcttcagacaactttggaaacacaccgctctttgaagctatcaagaatggacacgatcgtgttgcttcattacttgttaaggaaggtgccttcttgaagattgaaaatgctggtagcttcttgtgcacattggttacaaaaggggattcagatctactacgaaggctgttgtccaacggtattgatgcaaactccaaagattatgatcaccgaacaccgctccatgtagcggcttctcaaggattactcgcaatggcgagattgcttctgggagctggtgcttcagttttttcaaaggacagatggggaaatactccatttgatgaagctagactaagtggaaacaatcagttgatcaagcttctggaagaagcaaaatctgctcaaacatcggaaatccacagtgtttcacacgagatttcagaaaaaattcacctgcggaagtgtactgtataccctatccacccatgggagcctaaggacctcaggaaacatggtgttgttttatgggtacctacgagtatggaagagctcgttactgcagcttcggaacaactcaacttctcatcaggctcttgtattttatcagaagatgcaggtaaaattcttgatatagatatgatatctgatggtcagaagttgtacttgatcagtgaaacaacttga

SEQ ID NO: 21 - protein, 824 aa, Solanum tuberosum
MSMKRELGEERNGRDSPKEYKMNDLRDSMKSLRSTSRLAMMENELIADSTPSRFSRENVINGLRGLSQDFVIYPDDRWYKLWDKFILIWAIYSTFFTPMEFAFFKGLPRKLFLLDICGQIAFLVDIVIQFFVAYRDSQTYKMVYRRTPIALRYLKSHFILDVLSCMPWDNIFKASGRIEGVRYLLWIRLSRVRRVTDFFQKMEKDIRINYLFTRIIKLITVELYCTHTAACIFYFLATTLPAEKEGYTWIGSLTLGDYSYSNFREIDLWRRYITSLYFAIVTMATVGYGDIHAVNLREMIFVMVYVSFDMILGAYLIGNMTALIVKGSKTVRYRDKMTDLMNYMNRNRLGRDIRSQIKDHLRLQYESAYTDGAVLHDLPISIRAKISQTLYQSCIENIPLFRECSAEFISQIVTRVHEEFFLPGEVIMEQGHVVDQLYFVCDGVLVEVGIGEDGSQETVAHLEPNSSFGEISILCNIPQPYTVRVCELCRLIRIDKQSFSNILEIYFHDGRRILANLLEGKDLRVKQLESDITFHIGKQEADLALKVNSAAYHGDLHQLKSLIRAGADPNKKDYDGRSPLHLSASRGYEDISFFLVKEGIDLNASDNFGNTPLFEAIKNGHDRVASLLVKEGAFLKIENAGSFLCTLVTKGDSDLLRRLLSNGIDANSKDYDHRTPLHVAASQGLLAMARLLLGAGASVFSMDRWGNTPFDEARLSGNNQLIKLLEDAKSAQTSEFPSVSHEISEKKHPRKCTVFPVHPWEPKDLRKHGVVLWVPTSMEELVTAASEQLNFPSGSCILSEDAGKILDIDMISDGQKLYLISETT

SEQ ID NO: 22 - RNA, 2475 nt, Solanum tuberosum
  source 1..2475: mol_type = mRNA
atgtcgatgaagagagaattaggagaagagaggaatggaagagattcaccaaaagagtataagatgaatgatttgagagatagtatgaaatcgttgagaagtaccagtagattagcgatgatggagaacgagctaatagctgattcaactccttcgagatttagcagagaaaacgtcatcaatggcctcagaggcctttctcaagatttcgtcatttatcccgatgacaggtggtacaagctatgggataagtttatcctgatttgggcaatatattcaactttttttacaccaatggagtttgcattctttaaaggattgcccaggaaactatttcttttagacatttgtggtcaaatagcttttcttgtcgacatagtcatacaattttttgtagcctacagagatagtcagacatacaagatggtgtataggcgaacccctattgctcttcggtacttgaaatctcattttatcttggatgttctcagttgcatgccgtgggataacatttttaaggcttctggaagaattgaaggagtgagataccttttatggattaggttaagcagggtgcgcagggttactgactttttccagaagatggagaaagatattcgaatcaattatctcttcactaggattataaagcttatcactgttgaactttattgcacgcatacagcagcttgcatcttttactttttggcaactactctgcctgcagagaaagaaggctacacatggattggtagtttgaccctgggagattacagttattcaaactttagagagattgatctctggaggcggtacatcacttctctgtactttgcaattgtcactatggcaactgtgggttatggtgacatacatgcagtcaatctgagggaaatgatatttgtgatggtttatgtttcttttgatatgattcttggtgcttacttgatcggtaacatgacagcactgattgtcaagggatcaaaaactgtgcgatacagggacaaaatgacagatcttatgaactatatgaacagaaatagactcggaagggatattcgtagtcaaattaaagatcacttgcgattacaatatgaaagcgcttacactgatggagccgttctccatgaccttcccatctcaattcgtgccaagatatcccagactttatatcagtcttgcattgaaaatattcctctttttagggagtgctccgcagaattcataagtcaaattgtaactcgagtgcatgaagaatttttcctcccaggagaagtgatcatggaacaggggcatgtagtggaccaactttattttgtctgtgatggtgtcctggtagaagttggtataggagaagatgggtcacaagagacagtagcacatcttgagcctaatagctcgttcggagaaatatccattctttgcaacattccacaaccatatactgtccgtgtttgtgaactatgcagactcatacggattgataagcagtcattttcaaatattttggagatctattttcacgatggaagaagaatcttggcgaacttactagagggaaaggatcttcgtgtgaagcaactggagtcagatataacattccatattgggaaacaagaggctgatcttgctttgaaagttaatagtgcagcttatcatggtgacttacaccagctgaagagtcttattcgagctggagctgatcccaacaagaaagattatgatggaagatcacctctgcatctttcagcatccagaggatatgaagacatctcttttttccttgtcaaagaaggtatcgatctcaatgcttcagacaactttggaaacacaccgctctttgaagctatcaagaatggacacgatcgtgttgcttcattacttgttaaggaaggtgccttcttgaagattgaaaatgctggtagcttcttgtgtacgttggttacaaaaggggattcagatctactacgaaggctgttgtccaatggtattgatgcaaactccaaagattatgatcaccgaacaccgctccatgtagcggcttctcaaggattgcttgcgatggcgagattgcttctgggagctggtgcttcagttttttcaatggacagatggggaaatactccatttgatgaagctagactaagtggaaacaatcagttgatcaagcttctggaagacgcaaaatcagctcaaacatcagaattccccagtgtttcgcatgagatttcagaaaaaaagcacccacggaagtgtactgtattccctgtccacccatgggagcccaaggacctcaggaaacatggtgttgtactatgggtacctacgagtatggaagagctcgttactgcagcttcagaacaactcaacttcccatcaggctcttgtattttatcagaagatgcaggtaaaattcttgatatagatatgatatctgatggtcagaagttgtacttgatcagtgaaacaacttga

SEQ ID NO: 23 - protein, 846 aa, Arabidopsis thaliana
MGRLRRRQEIIDHEEEESNDDVSSDDGDLSLAKTFDWLKSSKDDDIKTKGDNKYDYIIDPKNRWYKAWEMFILVWAIYSSLFTPMEFGFFRGLPERLFVLDIVGQIAFLVDIVLQFFVAYRDTQTYRTVYKPTRIAFRYLKSHFLMDFIGCFPWDLIYKASGKHELVRYLLWIRLFRVRKVVEFFQRLEKDTRINYLFTRILKLLFVEVYCTHTAACIFYYLATTLPPENEGYTWIGSLKLGDYSYENFREIDLWKRYTTALYFAIVTMATVGYGDIHAVNLREMIFVMIYVSFDMVLGAYLIGNITALIVKGSNTERFRDKMNDLISFMNRKKLGRDLRSQITGHVRLQYDSHYTDTVMLQDIPASIRAKIAQLLYLPYIKKVPLFKGCSTEFINQIVIRLHEEYFLPGEVITEQGNVVDHLYFVCEGLLEALVTKTDGSEESVTLLGPHTSFGDISIICNISQPFTVRVCELCHLLRLDKQSFSNILEIYFHDGRTILNNIMEEKESNDRIKKLESDIVIHIGKQEAELALKVNSAAFQGDFYQLKSLIRSGADPNKTDYDGRSPLHLAACRGYEDITLFLIQEGVDVNLKDKFGHTPLFEAVKAGQEGVIGLLAKEGASFNLEDSGNFLCTTVAKGDSDFLKRLLSSGMDPNSEDYDHRTPLHVAASEGLFLMAKMLVEAGASVISKDRWGNSPLDEARLCGNKKLIKLLEDTKNAQSSIYPTSLRELQEERIERRKCTVFPFHPQEAKEERSRKHGVVVWIPSNLEKLIVTAAKELGLSDGASFVLLSEDQGRITDIDMISDGHKLYMISDTTDQTHPAFLYKVVMGRPRRSTRVDSRYPTS

SEQ ID NO: 24 - RNA, 2460 nt, Arabidopsis thaliana
  source 1..2460: mol_type = mRNA
atgggacgtctccggagacggcaagagataatagatcatgaagaagaggaatcaaacgacgacgtttcatcagatgatggagatctcagtttagccaaaacgtttgattggcttaaatcatcaaaagatgatgatattaaaaccaaaggtgataataaatatgattacatcattgatcccaaaaacaggtggtacaaggcatgggaaatgtttatattggtgtgggcaatatactcctcattgttcactcccatggagtttggtttcttccgcggtctgcctgagagactctttgtacttgacattgttggtcagatcgcgttcttggtcgatattgttcttcagttctttgttgcctatcgcgatactcagacctaccggactgtctacaaaccaacacgtattgctttccggtacttgaagtcgcattttctcatggatttcatcggttgcttcccttgggatcttatttataaggcatcagggaaacatgagttggtgaggtacttgttgtggataaggctatttcgggttcgcaaagtggttgagtttttccaaaggcttgagaaagacacaagaatcaactatctattcactagaatcttaaagctcttgttcgttgaagtttattgtactcacactgctgcttgtatcttctattacttggccaccactcttcctccggaaaacgaaggttacacttggatcggtagcttgaagctaggagactatagctacgagaatttccgagaaatcgatctatggaaacgttatactactgctctatacttcgccattgtcactatggcaactgtcggttatggagacattcacgcggtgaatctgagggaaatgatatttgtaatgatatatgtttcatttgatatggttctcggtgcttaccttattggtaacatcactgccttgattgtgaaaggttcaaacacagagaggttcagagataaaatgaatgatctcataagtttcatgaaccgcaaaaaactcgggagagaccttcgtagccagataactggtcatgttagattgcagtacgacagtcactacaccgacactgtcatgcttcaggacatcccagcatcaatccgcgccaagattgcgcaattattgtatctgccttacatcaaaaaagttcctctcttcaaaggctgctccacagagtttattaatcaaatagttataaggctccatgaagagtattttcttccaggagaagtaataacagagcaaggaaacgtcgtggatcatttgtatttcgtctgtgaaggcttactggaggctcttgttacaaaaacagatggatcagaagagagtgtgacgttacttgggcctcacacttcttttggagacatctccatcatttgcaacatttctcaacctttcactgttagggtttgtgagctatgccatcttttacgactcgataaacagtctttctcaaacatcctcgagatttattttcacgacggacgcacaatcttgaacaatattatggaggagaaggaatcaaatgataggataaagaagctagaatctgacatagtgattcacattgggaaacaagaagcagaacttgcattgaaagtaaacagtgcagctttccaaggagatttttaccagcttaagagcttaatccgatctggagccgatcctaacaaaaccgattacgatggaagatcaccgcttcatcttgcggcatgtagaggctatgaagacattacattattccttattcaagaaggtgttgatgtcaatctaaaagataagttcggacacacaccattgtttgaggctgtgaaagcaggacaagaaggagtgattggtttgcttgccaaagaaggagcctcctttaatttagaagattcaggaaacttcctttgcacgacagttgctaaaggcgactctgattttctcaagagattgctttcaagcggtatggacccaaacagtgaagattatgatcacagaacgccgcttcatgtcgcggcttctgaagggttattcttgatggctaaaatgttggttgaagctggagcaagcgttatttctaaagaccggtgggggaattctccgcttgatgaagctcgattgtgcggaaacaagaaactgattaagttactcgaagatacgaaaaatgctcagtcgtctatctacccgacaagcttgcgtgaattacaagaggagagaattgagagacggaaatgcacggtgtttccattccacccgcaagaggcgaaagaagagcgtagtagaaagcacggagttgtggtttggatcccaagcaatctcgagaaactcatagtaaccgctgcgaaagagctagggctatcggatggagcctcatttgtactattatccgaagaccaaggtcgtatcacagacattgatatgattagtgatggacacaaattgtatatgatcagtgatactactgatcaaaca

SEQ ID NO: 25 - RNA, 2460 nt, Arabidopsis thaliana
  source 1..2460: mol_type = mRNA
atgggacgtctccggagacggcaagagataatagatcatgaagaagaggaatcaaacgacgacgtttcatcaagaagaggaaaactcagtttagccaaaacgtttcggtggcttaaatcatcaaaaaaacggagaattaaaaccaaaggtaaaaataaatataaatacatcattaaacccaaaaacaggtggtacaaggcatgggaaatgtttatattggtgtgggcaatatactcctcattgttcactcccatggagtttggtttcttccgcggtctgcctgagagactctttgtacttgacattgttggtcagatcgcgttcttggtcgatattgttcttcagttctttgttgcctatcgcgatactcagacctaccggactgtctacaaaccaacacgtattgctttccggtacttgaagtcgcattttctcatggatttcatcggttgcttcccttgggatcttatttataaggcatcagggaaacatgagttggtgaggtacttgttgtggataaggctatttcgggttcgcaaagtggttgagtttttccaaaggcttgagaaagacacaagaatcaactatctattcactagaatcttaaagctcttgttcgttgaagtttattgtactcacactgctgcttgtatcttctattacttggccaccactcttcctccggaaaacgaaggttacacttggatcggtagcttgaagctaggagactatagctacgagaatttccgagaaatcgatctatggaaacgttatactactgctctatacttcgccattgtcactatggcaactgtcggttatggagacattcacgcggtgaatctgagggaaatgatatttgtaatgatatatgtttcatttgatatggttctcggtgcttaccttattggtaacatcactgccttgattgtgaaaggttcaaacacagagaggttcagagataaaatgaatgatctcataagtttcatgaaccgcaaaaaactcgggagagaccttcgtagccagataactggtcatgttagattgcagtacgacagtcactacaccgacactgtcatgcttcaggacatcccagcatcaatccgcgccaagattgcgcaattattgtatctgccttacatcaaaaaagttcctctcttcaaaggctgctccacagagtttattaatcaaatagttataaggctccatgaagagtattttcttccaggagaagtaataacagagcaaggaaacgtcgtggatcatttgtatttcgtctgtgaaggcttactggaggctcttgttacaaaaacagatggatcagaagagagtgtgacgttacttgggcctcacacttcttttggagacatctccatcatttgcaacatttctcaacctttcactgttagggtttgtgagctatgccatcttttacgactcgataaacagtctttctcaaacatcctcgagatttattttcacgacggacgcacaatcttgaacaatattatggaggagaaggaatcaaatgataggataaagaagctagaatctgacatagtgattcacattgggaaacaagaagcagaacttgcattgaaagtaaacagtgcagctttccaaggagatttttaccagcttaagagcttaatccgatctggagccgatcctaacaaaaccgattacgatggaagatcaccgcttcatcttgcggcatgtagaggctatgaagacattacattattccttattcaagaaggtgttgatgtcaatctaaaagataagttcggacacacaccattgtttgaggctgtgaaagcaggacaagaaggagtgattggtttgcttgccaaagaaggagcctcctttaatttagaagattcaggaaacttcctttgcacgacagttgctaaaggcgactctgattttctcaagagattgctttcaagcggtatggacccaaacagtgaagattatgatcacagaacgccgcttcatgtcgcggcttctgaagggttattcttgatggctaaaatgttggttgaagctggagcaagcgttatttctaaagaccggtgggggaattctccgcttgatgaagctcgattgtgcggaaacaagaaactgattaagttactcgaagatacgaaaaatgctcagtcgtctatctacccgacaagcttgcgtgaattacaagaggagagaattgagagacggaaatgcacggtgtttccattccacccgcaagaggcgaaagaagagcgtagtagaaagcacggagttgtggtttggatcccaagcaatctcgagaaactcatagtaaccgctgcgaaagagctagggctatcggatggagcctcatttgtactattatccgaagaccaaggtcgtatcacagacattgatatgattagtgatggacacaaattgtatatgatcagtgatactactgatcaaaca

SEQ ID NO: 26 - protein, 846 aa, Arabidopsis thaliana
MGRLRRRQEIIDHEEEESNDDVSSRRGKLSLAKTFRWLKSSKKRRIKTKGKNKYKYIIKPKNRWYKAWEMFILVWAIYSSLFTPMEFGFFRGLPERLFVLDIVGQIAFLVDIVLQFFVAYRDTQTYRTVYKPTRIAFRYLKSHFLMDFIGCFPWDLIYKASGKHELVRYLLWIRLFRVRKVVEFFQRLEKDTRINYLFTRILKLLFVEVYCTHTAACIFYYLATTLPPENEGYTWIGSLKLGDYSYENFREIDLWKRYTTALYFAIVTMATVGYGDIHAVNLREMIFVMIYVSFDMVLGAYLIGNITALIVKGSNTERFRDKMNDLISFMNRKKLGRDLRSQITGHVRLQYDSHYTDTVMLQDIPASIRAKIAQLLYLPYIKKVPLFKGCSTEFINQIVIRLHEEYFLPGEVITEQGNVVDHLYFVCEGLLEALVTKTDGSEESVTLLGPHTSFGDISIICNISQPFTVRVCELCHLLRLDKQSFSNILEIYFHDGRTILNNIMEEKESNDRIKKLESDIVIHIGKQEAELALKVNSAAFQGDFYQLKSLIRSGADPNKTDYDGRSPLHLAACRGYEDITLFLIQEGVDVNLKDKFGHTPLFEAVKAGQEGVIGLLAKEGASFNLEDSGNFLCTTVAKGDSDFLKRLLSSGMDPNSEDYDHRTPLHVAASEGLFLMAKMLVEAGASVISKDRWGNSPLDEARLCGNKKLIKLLEDTKNAQSSIYPTSLRELQEERIERRKCTVFPFHPQEAKEERSRKHGVVVWIPSNLEKLIVTAAKELGLSDGASFVLLSEDQGRITDIDMISDGHKLYMISDTTDQTHPAFLYKVVMGRPRRSTRVDSRYPTS

SEQ ID NO: 27 - RNA, 2442 nt, Arabidopsis thaliana
  source 1..2442: mol_type = mRNA
atgtcgatctcttggactcgaaatttcttcgaaagattctgcgtcgaggaatacaatatagacaccataaaacagagtagtttcctctctgccgatcttctaccatctcttggagccaggatcaaccaatctactaagctccgcaaacacataatctctcccaaaaacaggtggtacaaggcatgggaaatgtttatattggtgtgggcaatatactcctcattgttcactcccatggagtttggtttcttccgcggtctgcctgagagactctttgtacttgacattgttggtcagatcgcgttcttggtcgatattgttcttcagttctttgttgcctatcgcgatactcagacctaccggactgtctacaaaccaacacgtattgctttccggtacttgaagtcgcattttctcatggatttcatcggttgcttcccttgggatcttatttataaggcatcagggaaacatgagttggtgaggtacttgttgtggataaggctatttcgggttcgcaaagtggttgagtttttccaaaggcttgagaaagacacaagaatcaactatctattcactagaatcttaaagctcttgttcgttgaagtttattgtactcacactgctgcttgtatcttctattacttggccaccactcttcctccggaaaacgaaggttacacttggatcggtagcttgaagctaggagactatagctacgagaatttccgagaaatcgatctatggaaacgttatactactgctctatacttcgccattgtcactatggcaactgtcggttatggagacattcacgcggtgaatctgagggaaatgatatttgtaatgatatatgtttcatttgatatggttctcggtgcttaccttattggtaacatcactgccttgattgtgaaaggttcaaacacagagaggttcagagataaaatgaatgatctcataagtttcatgaaccgcaaaaaactcgggagagaccttcgtagccagataactggtcatgttagattgcagtacgacagtcactacaccgacactgtcatgcttcaggacatcccagcatcaatccgcgccaagattgcgcaattattgtatctgccttacatcaaaaaagttcctctcttcaaaggctgctccacagagtttattaatcaaatagttataaggctccatgaagagtattttcttccaggagaagtaataacagagcaaggaaacgtcgtggatcatttgtatttcgtctgtgaaggcttactggaggctcttgttacaaaaacagatggatcagaagagagtgtgacgttacttgggcctcacacttcttttggagacatctccatcatttgcaacatttctcaacctttcactgttagggtttgtgagctatgccatcttttacgactcgataaacagtctttctcaaacatcctcgagatttattttcacgacggacgcacaatcttgaacaatattatggaggagaaggaatcaaatgataggataaagaagctagaatctgacatagtgattcacattgggaaacaagaagcagaacttgcattgaaagtaaacagtgcagctttccaaggagatttttaccagcttaagagcttaatccgatctggagccgatcctaacaaaaccgattacgatggaagatcaccgcttcatcttgcggcatgtagaggctatgaagacattacattattccttattcaagaaggtgttgatgtcaatctaaaagataagttcggacacacaccattgtttgaggctgtgaaagcaggacaagaaggagtgattggtttgcttgccaaagaaggagcctcctttaatttagaagattcaggaaacttcctttgcacgacagttgctaaaggcgactctgattttctcaagagattgctttcaagcggtatggacccaaacagtgaagattatgatcacagaacgccgcttcatgtcgcggcttctgaagggttattcttgatggctaaaatgttggttgaagctggagcaagcgttatttctaaagaccggtgggggaattctccgcttgatgaagctcgattgtgcggaaacaagaaactgattaagttactcgaagatacgaaaaatgctcagtcgtctatctacccgacaagcttgcgtgaattacaagaggagagaattgagagacggaaatgcacggtgtttccattccacccgcaagaggcgaaagaagagcgtagtagaaagcacggagttgtggtttggatcccaagcaatctcgagaaactcatagtaaccgctgcgaaagagctagggctatcggatggagcctcatttgtactattatccgaagaccaaggtcgtatcacagacattgatatgattagtgatggacacaaattgtatatgatcagtgatactactgatcaaaca

SEQ ID NO: 28 - protein, 840 aa, Arabidopsis thaliana
MSISWTRNFFERFCVEEYNIDTIKQSSFLSADLLPSLGARINQSTKLRKHIISPKNRWYKAWEMFILVWAIYSSLFTPMEFGFFRGLPERLFVLDIVGQIAFLVDIVLQFFVAYRDTQTYRTVYKPTRIAFRYLKSHFLMDFIGCFPWDLIYKASGKHELVRYLLWIRLFRVRKVVEFFQRLEKDTRINYLFTRILKLLFVEVYCTHTAACIFYYLATTLPPENEGYTWIGSLKLGDYSYENFREIDLWKRYTTALYFAIVTMATVGYGDIHAVNLREMIFVMIYVSFDMVLGAYLIGNITALIVKGSNTERFRDKMNDLISFMNRKKLGRDLRSQITGHVRLQYDSHYTDTVMLQDIPASIRAKIAQLLYLPYIKKVPLFKGCSTEFINQIVIRLHEEYFLPGEVITEQGNVVDHLYFVCEGLLEALVTKTDGSEESVTLLGPHTSFGDISIICNISQPFTVRVCELCHLLRLDKQSFSNILEIYFHDGRTILNNIMEEKESNDRIKKLESDIVIHIGKQEAELALKVNSAAFQGDFYQLKSLIRSGADPNKTDYDGRSPLHLAACRGYEDITLFLIQEGVDVNLKDKFGHTPLFEAVKAGQEGVIGLLAKEGASFNLEDSGNFLCTTVAKGDSDFLKRLLSSGMDPNSEDYDHRTPLHVAASEGLFLMAKMLVEAGASVISKDRWGNSPLDEARLCGNKKLIKLLEDTKNAQSSIYPTSLRELQEERIERRKCTVFPFHPQEAKEERSRKHGVVVWIPSNLEKLIVTAAKELGLSDGASFVLLSEDQGRITDIDMISDGHKLYMISDTTDQTHPAFLYKVVMGRPRRSTRVDSRYPTS

SEQ ID NO: 29 - RNA, 2460 nt, Arabidopsis thaliana
  source 1..2460: mol_type = mRNA
atgggacgtctccggagacggcaagagataatagatcatgaagaagaggaatcaaacgacgacgtttcatcagatgatggagatctcagtttagccgagacgtttgattggcttgattcatcagaggacgatgatattgaaaccgatggtgataatgattatgattacatcattgatcccaaaaacaggtggtacaaggcatgggaaatgtttatattggtgtgggcaatatactcctcattgttcactcccatggagtttggtttcttccgcggtctgcctgagagactctttgtacttgacattgttggtcagatcgcgttcttggtcgatattgttcttcagttctttgttgcctatcgcgatactcagacctaccggactgtctacaaaccaacacgtattgctttccggtacttgaagtcgcattttctcatggatttcatcggttgcttcccttgggatcttatttataaggcatcagggaaacatgagttggtgaggtacttgttgtggataaggctatttcgggttcgcaaagtggttgagtttttccaaaggcttgagaaagacacaagaatcaactatctattcactagaatcttaaagctcttgttcgttgaagtttattgtactcacactgctgcttgtatcttctattacttggccaccactcttcctccggaaaacgaaggttacacttggatcggtagcttgaagctaggagactatagctacgagaatttccgagaaatcgatctatggaaacgttatactactgctctatacttcgccattgtcactatggcaactgtcggttatggagacattcacgcggtgaatctgagggaaatgatatttgtaatgatatatgtttcatttgatatggttctcggtgcttaccttattggtaacatcactgccttgattgtgaaaggttcaaacacagagaggttcagagataaaatgaatgatctcataagtttcatgaaccgcaaaaaactcgggagagaccttcgtagccagataactggtcatgttagattgcagtacgacagtcactacaccgacactgtcatgcttcaggacatcccagcatcaatccgcgccaagattgcgcaattattgtatctgccttacatcaaaaaagttcctctcttcaaaggctgctccacagagtttattaatcaaatagttataaggctccatgaagagtattttcttccaggagaagtaataacagagcaaggaaacgtcgtggatcatttgtatttcgtctgtgaaggcttactggaggctcttgttacaaaaacagatggatcagaagagagtgtgacgttacttgggcctcacacttcttttggagacatctccatcatttgcaacatttctcaacctttcactgttagggtttgtgagctatgccatcttttacgactcgataaacagtctttctcaaacatcctcgagatttattttcacgacggacgcacaatcttgaacaatattatggaggagaaggaatcaaatgataggataaagaagctagaatctgacatagtgattcacattgggaaacaagaagcagaacttgcattgaaagtaaacagtgcagctttccaaggagatttttaccagcttaagagcttaatccgatctggagccgatcctaacaaaaccgattacgatggaagatcaccgcttcatcttgcggcatgtagaggctatgaagacattacattattccttattcaagaaggtgttgatgtcaatctaaaagataagttcggacacacaccattgtttgaggctgtgaaagcaggacaagaaggagtgattggtttgcttgccaaagaaggagcctcctttaatttagaagattcaggaaacttcctttgcacgacagttgctaaaggcgactctgattttctcaagagattgctttcaagcggtatggacccaaacagtgaagattatgatcacagaacgccgcttcatgtcgcggcttctgaagggttattcttgatggctaaaatgttggttgaagctggagcaagcgttatttctaaagaccggtgggggaattctccgcttgatgaagctcgattgtgcggaaacaagaaactgattaagttactcgaagatacgaaaaatgctcagtcgtctatctacccgacaagcttgcgtgaattacaagaggagagaattgagagacggaaatgcacggtgtttccattccacccgcaagaggcgaaagaagagcgtagtagaaagcacggagttgtggtttggatcccaagcaatctcgagaaactcatagtaaccgctgcgaaagagctagggctatcggatggagcctcatttgtactattatccgaagaccaaggtcgtatcacagacattgatatgattagtgatggacacaaattgtatatgatcagtgatactactgatcaaaca

SEQ ID NO: 30 - protein, 820 aa, Arabidopsis thaliana
MGRLRRRQEIIDHEEEESNDDVSSDDGDLSLAETFDWLDSSEDDDIETDGDNDYDYIIDPKNRWYKAWEMFILVWAIYSSLFTPMEFGFFRGLPERLFVLDIVGQIAFLVDIVLQFFVAYRDTQTYRTVYKPTRIAFRYLKSHFLMDFIGCFPWDLIYKASGKHELVRYLLWIRLFRVRKVVEFFQRLEKDTRINYLFTRILKLLFVEVYCTHTAACIFYYLATTLPPENEGYTWIGSLKLGDYSYENFREIDLWKRYTTALYFAIVTMATVGYGDIHAVNLREMIFVMIYVSFDMVLGAYLIGNITALIVKGSNTERFRDKMNDLISFMNRKKLGRDLRSQITGHVRLQYDSHYTDTVMLQDIPASIRAKIAQLLYLPYIKKVPLFKGCSTEFINQIVIRLHEEYFLPGEVITEQGNVVDHLYFVCEGLLEALVTKTDGSEESVTLLGPHTSFGDISIICNISQPFTVRVCELCHLLRLDKQSFSNILEIYFHDGRTILNNIMEEKESNDRIKKLESDIVIHIGKQEAELALKVNSAAFQGDFYQLKSLIRSGADPNKTDYDGRSPLHLAACRGYEDITLFLIQEGVDVNLKDKFGHTPLFEAVKAGQEGVIGLLAKEGASFNLEDSGNFLCTTVAKGDSDFLKRLLSSGMDPNSEDYDHRTPLHVAASEGLFLMAKMLVEAGASVISKDRWGNSPLDEARLCGNKKLIKLLEDTKNAQSSIYPTSLRELQEERIERRKCTVFPFHPQEAKEERSRKHGVVVWIPSNLEKLIVTAAKELGLSDGASFVLLSEDQGRITDIDMISDGHKLYMISDTTDQT

SEQ ID NO: 31 - protein, 47 aa, Brassica napus
DDVSSRRGKLSLAETFRWLDSSEHRRIETDGHNDYKYIIHPKNRWYK

SEQ ID NO: 32 - protein, 51 aa, Brassica napus
DDVSRRRGGFSLAESFRWLDSPEHLKDDSDGPNEYPWIIKPSISRWYKAWE

SEQ ID NO: 33 - protein, 47 aa, Terenaya hassleriana
SSSSSRGGSRFSLIDRIRWLDLSDRRKIDPDAPSGGFFIDPNTRSYK

SEQ ID NO: 34 - protein, 53 aa, Elaeis guineensis
EEPIASSRGIRLFLLTSEFALGPLRRRRATSQEKLLERFVIEPDNRWYQLWTR

SEQ ID NO: 35 - protein, 50 aa, Raphanus sativus
DDVSMRRGRFSLAESFRWLDSPEHLKNDSDCPNEHPWIINPSNRWYKAWE

SEQ ID NO: 36 - protein, 62 aa, Capsicum annuum
EDFRDSMKSLRSSSRLAMMENELATDSTNSRFSRENVINGLKGLSQGFIVYPDDRWYKLWKK

SEQ ID NO: 37 - protein, 60 aa, Hibiscus syriacus
EEFREGTIASRGSRFNLLAAELGLAAARARKNFSRQSLLDGIKDGLSIHPDNRWYRTWTK

SEQ ID NO: 38 - protein, 62 aa, Triticum aestivum
EEVRDRLQSSRNSRLALFGSDLRLGPRRRRPPRRPAVDGEDGFFHDHIILPDNKWYLLWTKF

SEQ ID NO: 39 - protein, 58 aa, Oryza sativa
VDVVRDRIGSSRGSRLALFGSDLRLGRFRPRRRRVAPVDGDDGIFQDFVIDPDNKWYR

SEQ ID NO: 40 - protein, 46 aa, Gynandropsis gynandra
GDVSTVGRRLSLVDKIRWIDLSDRRKFDREFLSGRFIIDPTTRWYK

SEQ ID NO: 41 - protein, 65 aa, Zea mays
EVVRDHIASSRGSRLALFGSELRLGRFRPRRRRRLPLAGEGAAEGFFHGLVIHPDNKWYRLWTKF

SEQ ID NO: 42 - protein, 59 aa, Panicum miliaceum
EVVRDHIASSRGSRLALFGSDLRLGRFRPRRRRREGGAEGFFQDLVIHPDNRWYRLWTK

SEQ ID NO: 43 - protein, 67 aa, Sorghum bicolor
EEVRDHIASSRGSRLALFGSDLRLGRFRPRRRTRRRLLAGEVGAVEGFFHGLVIHPDNKWYRLWTKF

SEQ ID NO: 44 - protein, 48 aa, Brassica napus
DDVSRRRGGFSLAESFRWLDSPEHLKDDSDGPNEYPWIIKPSISRWYL

SEQ ID NO: 45 - protein, 49 aa, Elaeis guineensis
EEPIASSRGIRLFLLTSEFALGPLRRRRATSQEKLLERFVIEPDNRWYQ

SEQ ID NO: 46 - protein, 58 aa, Capsicum annuum
EDFRDSMKSLRSSSRLAMMENELATDSTNSRFSRENVINGEKGLSQGFIVYPDDRWYK

SEQ ID NO: 47 - protein, 57 aa, Triticum aestivum
EEVRDRLQSSRNSRLALFGSDLRLGPRRRRPPRRPAVDGEDGFFHDHIILPDNKWYL

SEQ ID NO: 48 - protein, 41 aa, Arabidopsis thaliana
IDTIKQSSFLSADLLPSLGARINQSTKLRKHIISPFNPRYR

SEQ ID NO: 49 - protein, 47 aa, Arabidopsis thaliana; identical to SEQ ID NO: 31

SEQ ID NO: 50 - protein, 46 aa, Arabidopsis thaliana
DDVSSRRGKLSLAKTFRWLKSSKKRRIKTGKNKYKYIIKPKNRWYK

SEQ ID NO: 51 - protein, 47 aa, Arabidopsis thaliana
DDVSSDDGDLSLAETFDWLDSSEDDDIETDGDNDYDYIIDPKNRWYK

SEQ ID NO: 52 - protein, 47 aa, Arabidopsis thaliana
DDVSSAAGALSLAATFAWLASSAAAAIATAGANAYAYIIAPKNRWYK

SEQ ID NO: 53 - protein, 47 aa, Arabidopsis thaliana
DDVSSDDGDLSLAKTFDWLKSSKDDDIKTKGDNKYDYIIDPKNRWYK

SEQ ID NO: 54 - protein, 6 aa, Arabidopsis thaliana
TXGYGD

SEQ ID NO: 55 - protein, 58 aa, Oryza sativa
VDVVRDRIGSSRGSRLALFGSDLRLGDFRPRRRRVAPVDGDDGIFQDFVIDPDNKWYR